(19) United States
(12) Patent Application Publication
(10) Pub. No.: US 2008/0233655 A1
(43) Pub. Date: Sep. 25, 2008

(54) Screening For Lysosomal Storage Disease Status

(75) Inventors: Peter Meikle, South Australia (AU); John Hopwood, South Australia (AU); Maria Fuller, South Australia (AU); Phillip Whitfield, Liverpool (GB); Peter Sharp, South Australia (AU)

Correspondence Address: COLEMAN SUDOL SAPONE, P.C., 714 COLORADO AVENUE, BRIDGE PORT, CT, 06605-1601, US

(73) Assignee: Children, Youth and Women's Health Service, North Adelaide (AU)

(21) Appl. No.: 10/594,699

(22) PCT Filed: Mar. 31, 2005
(86) PCT No.: PCT/AU05/00461

§ 371 Date: Sep. 28, 2006

(30) Foreign Application Priority Data
Mar. 31, 2004 (AU) 2004901726

(51) Int. Cl. G01N 33/00

(52) U.S. Cl. 436/63

(57) ABSTRACT

A method of ascertaining the LSD (Lysosomal storage disorder) status of an individual comprising taking a tissue or body fluid sample from the individual and estimating a level in the sample of each of three or more compound indicators. The indicators reflect the level of respectively each of three or more lipid containing storage associated compounds. The levels are used to calculate an LSD index number which is then compared with a standard to provide an assessment of the LSD status of the individual. The indicator compounds are conveniently phospholipids, glycolipids or lipopolysaccharide species measured by mass spectrometry. The method may be used to ascertain the nature of the disorder from which the individual stuffers, and its severity. It may also be used to monitor the progress of treatment and to ascertain the prospects of an individual contracting an LSD by providing a subclinical indicators for the condition.

### FIELD OF THE INVENTION

This invention relates to screening to ascertain the nature or status of lysosomal storage disorders (LSD) and in particular by the use of lipid containing storage associated compounds

### BACKGROUND OF THE INVENTION

Most lysosomal storage disorders (LSD) are inherited in an autosomal recessive manner with the exception of Fabry disease, Danon disease and mucopolysaccharidosis (MPS) type II, which display X-linked recessive inheritance. Some LSD have been classified into clinical subtypes (such as the Hurler/Scheie variants of MPS I, or the infantile/juvenile/adult onset forms of Pompe disease), but it is clear that most LSD have a broad continuum of clinical severity and age of presentation. With the advent of molecular biology/genetics and the characterisation of many of the LSD genes, it is now recognised that the range of severity may, in part, be ascribed to different mutations within the same gene. However, genotype/phenotype correlations do not always hold and other factors including genetic background and environmental factors, presumably play a role in disease progression.

LSD are rare disorders with incidences ranging from about 1:50,000 births to less than 1:4,000,000 births (1). However, when considered as a group, the combined incidence is substantially higher. We have previously estimated the prevalence of LSD in Australia to be 1:7,700 births, excluding the neuronal ceroid lipofuscinoses. The prevalence of this latter group of LSD has been reported to be as high as 1 per 12,500 births in the United States (2). In Finland, incidence values of 1 per 13,000 births for infantile and 1 per 21,000 births for juvenile forms have been reported (3). Clearly, the neuronal ceroid lipofuscinoses will contribute significantly to the overall prevalence of LSD. It is equally certain that additional LSD will be identified as our understanding of lysosomal biology and the clinical manifestations resulting from lysosomal dysfunction improve. A conservative estimate of the prevalence of LSD in the Australian population would be 1 in 5,000 births.

Inborn errors of metabolism causing lysosomal storage have well-recognised effects on neuronal function. In many of the LSD almost all patients develop central nervous system (CNS) dysfunction while in a few disorders such as MPS IVA and MPS VI there are no reports of CNS involvement. In a number of other disorders, notably Gaucher disease, Niemann-Pick disease, MPS I and MPS II, the range of clinical severity spans individuals with no CNS involvement to those with severe CNS pathology. Notwithstanding the diverse clinical manifestations within LSD, the majority of patients will develop CNS disease.

One of the main determining factors of LSD severity is the residual activity of the affected enzyme. Kinetic models that describe correlations between residual enzyme activity and the turnover rate of its substrate have been proposed (4). Such a mathematical model has been tested in skin fibroblasts and residual activity of β-hexosaminidase A and arylsulphatase A correlated well with substrate turnover (5). However, for many LSD residual enzyme activity is difficult to measure accurately and even when such measurements can be performed they are not always reflective of disease severity, especially CNS pathology. We propose that the level of stored substrates in particular cells or tissues in these disorders, as well as perhaps the levels of secondary metabolites, will reflect disease severity and is likely to yield additional information about the pathophysiology in LSD. The key in determining the absence or presence of CNS pathology lies in understanding the pathogenic process of LSD, which at present is poorly understood.

Unless the context requires otherwise, the word “comprise,” or variations such as “comprises” or “comprising” mean the inclusion of a stated element or integer or group of elements or integers, but not the exclusion of any other element or integer or group of elements or integers.

### SUMMARY OF THE INVENTION

It has been found that use of estimates of the relative levels of LSD (Lysosomal Storage Disorder) storage associated compounds in body tissues or fluids can be used to assess the LSD status of an individual.

In a first broad form of a first aspect the invention could be said to reside in a method of assessing an LSD status of an individual the method comprising the steps of,
taking a tissue or body fluid sample from the individual,
estimating a level in the sample of each of three or more compound indicators, said indicators being indicative of the level of respectively each of three or more lipid containing storage associated compounds,
calculating an LSD index number using all of said compound indicators,
and comparing the LSD index number of the sample with a standard to provide an assessment of the LSD status of the individual.

In a first broad form of a second aspect the invention could be said to reside in a method of assessing an LSD status of an individual the method comprising the steps of,
taking a tissue or body fluid sample from the individual,
estimating a level in the sample of each of two or more compound indicators being indicative of the level respectively of each of two or more lipid containing storage associated compounds,
calculating an LSD index number using all of said compound indicators,
and comparing the LSD index number of the sample with a standard to provide an assessment of the LSD status of the individual,
   the two or more storage associated compounds selected to discriminate between an LSD individual from a non-LSD individual with an acceptable confidence level.

In a first broad form of a third aspect the invention could be said to reside in a method for screening for the status of two or more LSDs in an individual,
taking a single tissue or body fluid sample from the individual,
estimating a level in the sample of each three or more compound indicators being indicative of the concentration respectively of each of three or more lipid containing storage associated compounds,
calculating a first LSD index number using a first set of two or more of said compound indicators and comparing the first LSD index number of the sample with a first control indicator to provide an assessment of the LSD status of the first LSD,
and calculating a second LSD index number using a second set of two or more of said compound indicators and comparing the second LSD index number of the individual with a second standard to provide an assessment of the LSD status of the second LSD in the individual.

In a first broad form of a fourth form the invention might be said to reside in a method of developing a diagnostic method comprising the steps of
taking a first group of LSD samples one each from a plurality of LSD individuals affected by one type of LSD,
taking a second group of control samples one each from a plurality of control individuals not affected by LSD
the sample being of a tissue or body fluid of the control individuals and LSD group of individuals
interrogating the first group of samples by mass spectrometry for first levels of a plurality of indicators of respective storage associated compounds,
interrogating the second group of samples by mass spectrometry for second levels of the plurality of indicators of respective storage associated compounds,
the storage associated compounds selected from the class of compounds consisting of the group glycolipids and phospholipids,
comparing the first levels with the second levels
   identifying a first group of storage associated compound which are shown as having increased levels of indicators in the first LSD group compared to the control group, identifying a second group of storage associated compounds which are shows as having decreased levels of indicators in the LSD group compared to the control group,
formulating a combination of two or more of the first and/or second group of indicators by which to calculate and index number whereby to distinguish LSD samples from control samples, and preferably
preparing a standard being a scale of index numbers reflective of the severity of the LSD.

### BRIEF DESCRIPTION OF THE DRAWINGS

FIG. 1. Glycolipid levels in Dried Blood Spots. Box plots showing the relative levels of glucosylceramide (panel A) and lactosylceramide (panel B) in dried blood spots from control (1), Gaucher patients on enzyme therapy (2) and Gaucher patients not on therapy (3). The centre bar shows the median value, the box denotes the 25^{th} and 75^{th} centiles and the upper and lower bars represent the range. Open circles and stars represent outliers and extreme outliers respectively. N=the number of samples in each group.

FIG. 2. Glycolipid levels in Dried Blood Spot. Box plots showing the relative levels of ceramide (panel A) and sphingomyelin (panel B) in dried blood spots from control (1), Gaucher patients on enzyme therapy (2) and Gaucher patients not on therapy (3). The centre bar shows the median value, the box denotes the 25^{th} and 75^{th} centiles and the upper and lower bars represent the range. Open circles and stars represent outliers and extreme outliers respectively. N=the number of samples in each group.

FIG. 3. Glycolipid Ratios in Dried Blood Spots. Box plots showing the ratios of glucosylceramide to lactosylceramide (panel A) and ceramide to sphingomyelin (panel B) in dried blood spots from control (1), Gaucher patients on enzyme therapy (2) and Gaucher patients not on therapy (3). The centre bar shows the median value, the box denotes the 25^{th} and 75^{th} centiles and the upper and lower bars represent the range. Open circles and stars represent outliers and extreme outliers respectively. N=the number of samples in each group.

FIG. 4. Glycolipid Analysis in Dried Blood Spots. Box plots showing the ratio of (glucosylceramide×ceramide)/(lactosylceramide×sphingomyelin) (panel A) and a discriminate function of the same four analytes (panel B) in dried blood spots from control (1), Gaucher patients on enzyme therapy (2) and Gaucher patients not on therapy (3). The centre bar shows the median value, the box denotes the 25^{th} and 75^{th} centiles and the upper and lower bars represent the range. Open circles and stars represent outliers and extreme outliers respectively. N=the number of samples in each group.

FIG. 5. Relative lipid levels in dried blood spots from treated and untreated Gaucher disease patients. Relative glucosylceramide (panel A) and ceramide (panel B) were determined in dried blood spots from patients that were either untreated or had been receiving enzyme replacement therapy for up to 130 months. The shaded area shows the normal range for each analyte.

FIG. 6. Relative lipid ratios in dried blood spots from treated and untreated Gaucher disease patients. The ratio of (glucosylceramide×ceramide)/(lactosylceramide×sphingomyelin) (panel A) and a discriminate function of the same four analytes (panel B) were determined in dried blood spots from patients that were either untreated or had been receiving enzyme replacement therapy for up to 130 months. The shaded area shows the normal range for each ratio or function.

FIG. 7. Correlation between relative lipid levels in dried blood spots from treated and untreated Gaucher disease patients and chitotriosidase values. Glucosylceramide (panel A) and ceramide (panel B) were determined in dried blood spots from patients that were either untreated or had been receiving enzyme replacement therapy for up to 130 months. The lipid levels were related to the chitotriosidase levels determined in the same patients at the same time.

FIG. 8. Correlation between relative lipid ratios in dried blood spots from treated and untreated Gaucher disease patients and chitotriosidase values. Glucosylceramide:lactosylceramide ratio (panel A) and ceramide:sphingomyelin ratio (panel B) were determined in dried blood spots from patients that were either untreated or had been receiving enzyme replacement therapy for up to 130 months. The lipid levels were related to the chitotriosidase levels determined in the same patients at the same time.

FIG. 9. Correlation between relative lipid ratios in dried blood spots from treated and untreated Gaucher disease patients and chitotriosidase values. The ratio of (glucosylceramide×ceramide)/(lactosylceramide×sphingomyelin) (panel A) and a discriminate function of the same four analytes (panel B) were determined in dried blood spots from patients that were either untreated or had been receiving enzyme replacement therapy for up to 130 months. The lipid levels were related to the chitotriosidase levels determined in the same patients at the same time.

FIG. 10. Lipid concentrations in urine from controls, Fabry and Fabry heterozygotes. Urine samples (1.5 mL) were extracted with CHCl₃ by the method of Bligh/Dyer. Lipids were analysed by tandem mass spectrometry as described previously. The box plots show the median levels of each lipid type (centre bar), the 25^{th} and 75^{th} centiles (boxes) and the upper and lower limits (upper and lower bars). The circles and stars represent outliers and extreme outliers respectively.

FIG. 11. Lactosylceramide and trihexosylceramide concentrations in urine from controls, Fabry and Fabry heterozygotes. Urine samples (1.5 mL) were extracted with CHCl₃ using the method of Bligh/Dyer. Lipids were analysed by tandem mass spectrometry as described previously. The scatter plots show the relationship between the LC and CTH species. Fabry Het (affected) patients were heterozygotes who had been diagnosed with clinical symptoms of Fabry disease; clinical details were not available for the other heterozygotes. Two of the Fabry patients were known to have undergone renal transplants (Fabry (RT)).

FIG. 12. Lipid ratios in urine from controls, Fabry and Fabry heterozygotes. Urine samples (1.5 mL) were extracted with CHCl₃ using the method of Bligh/Dyer. Lipids were analysed by tandem mass spectrometry as described previously. Each lipid was corrected for the total PC concentration in that sample. The box plots show the median levels of each corrected lipid type (centre bar), the 25^{th} and 75^{th} centiles (boxes), and the upper and lower limits (upper and lower bars). The circles and stars represent outliers and extreme outliers respectively.

FIG. 13. Individual lipid species in urine from controls, Fabry and Fabry heterozygotes. Urine samples (1.5 mL) were extracted with CHCl₃ using the method of Bligh/Dyer. Lipids were analysed by tandem mass spectrometry as described previously. Each lipid species was corrected for the total PC concentration in that sample. The box plots show the median levels of each corrected lipid species (centre bar), the 25^{th} and 75^{th} centiles (boxes), and the upper and lower limits (upper and lower bars). The circles and stars represent outliers and extreme outliers respectively.

FIG. 14. Selected lipid species concentrations in urine from controls, Fabry and Fabry heterozygotes. Urine samples (1.5 mL) were extracted with CHCl₃ using the method of Bligh/Dyer. Lipids were analysed by tandem mass spectrometry as described previously. The scatter plots show the relationship between the lipid species. Fabry het (affected) patients were heterozygotes who had been diagnosed with clinical symptoms of Fabry disease; clinical details were not available for the other heterozygotes. Two of the Fabry patients were known to have undergone renal transplants (Fabry (RT)).

FIG. 15. Selected lipids and proteins in urine from controls, Fabry and Fabry heterozygotes. Urine samples (1.5 mL) were extracted with CHCl₃ using the method of Bligh/Dyer. Lipids were analysed by tandem mass spectrometry as described previously. The scatter plots show the relationship between the lipid ratios and saposin C. Fabry bet (affected) patients were heterozygotes who had been diagnosed with clinical symptoms of Fabry disease; clinical details were not available for the other heterozygotes. Two of the Fabry patients were known to have undergone renal transplants (Fabry (RT)).
Ratio 4=(LC C24:1*CTH C24:1)/(GC C24:0*SM C24:0) all species corrected for PC.

FIG. 16. Individual PC species in urine from controls, Fabry and Fabry heterozygotes. Urine samples (1.5 mL) were extracted with CHCl₃ using the method of Bligh/Dyer. Lipids were analysed by tandem mass spectrometry as described previously. Each lipid species was corrected for the total PC concentration in that sample. The box plots show the median levels of each corrected lipid species (centre bar), the 25^{th} and 75^{th} centiles (boxes), and the upper and lower limits (upper and lower bars). The circles and stars represent outliers and extreme outliers respectively.

FIG. 17. Lipid concentrations in plasma from controls, Fabry and Fabry heterozygotes. Plasma samples (100 μL) were extracted with CHCl₃ using the method of Folsch. Lipids were analysed by tandem mass spectrometry as described previously. The box plots show the median levels of each lipid type (centre bar), the 25^{th} and 75^{th} centiles (boxes), and the upper and lower limits (upper and lower bars). The circles and stars represent outliers and extreme outliers respectively.

FIG. 18. Lipid species in plasma from controls, Fabry and Fabry heterozygotes. Plasma samples (100 μL) were extracted with CHCl₃ using the method of Folsch. Lipids were analysed by tandem mass spectrometry as described previously. The scatter plots show the relationship between the different lipid species.

FIG. 19. Lipid concentrations in whole blood from controls, Fabry and Fabry heterozygotes. Dried blood spots (2×3 mm) were extracted with isopropanol and the lipids were analysed by tandem mass spectrometry as described previously. The box plots show the median levels of each lipid type (centre bar), the 25^{th} and 75^{th} centiles (boxes), and the upper and lower limits (upper and lower bars). The circles and stars represent outliers and extreme outliers respectively.

FIG. 20. Lipid species in whole blood from controls, Fabry and Fabry heterozygotes. Dried blood spots (2×3 mm) were extracted with isopropanol and the lipids were analysed by tandem mass spectrometry as described previously. The box plots show the median levels of each lipid species (centre bar), the 25^{th} and 75^{th} centiles (boxes), and the upper and lower limits (upper and lower bars). The circles and stars represent outliers and extreme outliers respectively.

FIG. 21. CTH species in whole blood from controls, Fabry and Fabry heterozygotes. Dried blood spots (2×3 mm) were extracted with isopropanol and the lipids were analysed by tandem mass spectrometry as described previously. The box plots show the median levels of each CTH species (centre bar), the 25^{th} and 75^{th} centiles (boxes), and the upper and lower limits (upper and lower bars). The circles and stars represent outliers and extreme outliers respectively.

FIG. 22. Lipid species in whole blood from controls, Fabry and Fabry heterozygotes. Dried blood spots (2×3 mm) were extracted with isopropanol and the lipids were analysed by tandem mass spectrometry as described previously. The scatter plots show the relationship between the different lipid species.

FIG. 23. Plasma CTH levels in controls, Fabry hemizygotes, Fabry hemizygotes on ERT, Fabry heterozygotes and Fabry heterozygotes on ERT. The bar represents the median value, the box represents the 25^{th} to 75^{th} centiles and the upper and lower bars represent the range. Circles and stars represent outliers and extreme outliers, respectively. N=sample numbers in each group.

FIG. 24. Plasma lipid levels in controls, Fabry hemizygotes, Fabry hemizygotes on ERT, Fabry heterozygotes and Fabry heterozygotes on ERT. The bar represents the median value, the box represents the 25^{th} to 75^{th} centiles and the upper and lower bars represent the range. Circles and stars represent outliers and extreme outliers, respectively. N=sample numbers in each group.

FIG. 25. Plasma lipid levels in controls, Fabry hemizygotes, Fabry hemizygotes on ERT, Fabry heterozygotes and Fabry heterozygotes on ERT.

FIG. 26. Urine lipid levels in controls, Fabry hemizygotes, Fabry hemizygotes on ERT, Fabry heterozygotes and Fabry heterozygotes on ERT. The bar represents the median value, the box represents the 25^{th} to 75^{th} centiles and the upper and lower bars represent the range. Circles and stars represent outliers and extreme outliers, respectively. N=sample numbers in each group.

FIG. 27. Urine lipid ratios in controls, Fabry hemizygotes, Fabry hemizygotes on ERT, Fabry heterozygotes and Fabry heterozygotes on ERT.

### DETAILED DESCRIPTION OF THE ILLUSTRATED AND EXEMPLIFIED EMBODIMENTS OF THE INVENTION

Lysosomes are organelles in eukaryotic cells that function in the degradation of macromolecules, including glycosphingolipids, glycogen, mucopolysaccharides, oligosaccharides, aminoglycans, phospholipids and glycoproteins, into component parts that can be reused in biosynthetic pathways or discharged by cells as waste. The metabolism of exo- and endogenous high molecular weight compounds normally occurs in the lysosomes, and the process is normally regulated in a stepwise process by degradation enzymes. However, when a lysosomal enzyme is not present in the lysosome or does not function properly, the enzymes specific macromolecular substrate accumulates in the lyosome as “storage material” causing a variety of diseases, collectively known as lysosomal storage diseases. In each of these diseases, lysosomes are unable to degrade a specific compound or group of compounds because the enzyme that catalyzes a specific degradation reaction is missing from the lysosome or is present in low concentrations or has been altered.

The field of lysosomal storage disorders is quite active and new LSD are still being found. The present invention is intended to include those that are found from time to time as well as the categories of LSD selected from the group consisting of mucopolysaccharidases (MPSs), lipidoses, glycogenoses, oligosaccharidoses and neuronal ceroid lipofuscinoses. A listing of many of the LSD currently known and the defective enzymes are listed below in table A. It will be understood that the LSD listed therein are encompassed by the present invention.

TABLE A

| Disease | Clinical Phenotype | Enzyme Deficiency |
|---|---|---|
| Aspartylglucosaminuria | | Aspartylglucosaminidase |
| Cholesterol ester | Wolman disease | Acid lipase |
| storage disease | | |
| Cystinosis | | Cystine transporter |
| Fabry disease | Fabry disease | α-Galactosidase A |
| Farber Lipogranulomatosis | Farber disease | Acid ceramidase |
| Fucosidosis | | α-L-Fucosidase |
| Galactosialidosis types I/II | | Protective protein |
| Gaucher disease types I/II/III | Gaucher disease | Glucocerebrosidase |
| | | (β-glucosidase) |
| Globoid cell leucodystrophy | Krabbe disease | Galactocerebrosidase |
| Glycogen storage disease II | Pompe disease | α-Glucosidase |
| GM1-Gangliosidosis | | β-GalactosidaSe |
| types I/II/III | | |
| GM2-Gangliosidosis type I | Tay Sachs disease | α-Hexosaminidase A |
| GM2-Gangliosidosis type II | Sandhoff disease | α-Hexosaminidase A & B |
| GM2-Gangliosidosis | | GM2-activator deficiency |
| α-Mannosidosis types I/II | | α-D-Mannosidase |
| β-Mannosidosis | | β-D-Mannosidase |
| Metachromatic leucodystrophy | | Arylsulphatase A |
| Metachromatic leucodystrophy | | Saposin B |
| Mucolipidosis type I | Sialidosis types I/II | Neuramindase |
| Mucolipidosis types II/III | I-cell disease; | Phosphotransferase |
| | pseudo-Hurler | |
| | polydystrophy | |
| Mucolipidosis type IIIC | pseudo-Hurler | Phosphotransferase γ-subunit |
| | polydystrophy | |
| Mucolipidosis type IV | | Unknown |
| Mucopolysaccharidosis type I | Hurler syndrome; | α-L-Iduronidase |
| | Scheie syndrome | |
| Mucopolysaccharidosis type II | Hunter syndrome | Iduronate-2-sulphatase |
| Mucopolysaccharidosis type | Sanfilippo syndrome | Heparan-N-sulphatase |
| IIIA | | |
| Mucopolysaccharidosis type | Sanfilippo syndrome | α-N-Acetylglucosaminidase |
| IIIB | | |
| Mucopolysaccharidosis type | Sanfilippo syndrome | AcetylCoA:N-acetyltransferase |
| IIIC | | |
| Mucopolysaccharidosis type | Sanfilippo syndrome | N-Acetylglucosamine 6- |
| IIID | | sulphatase |
| Mucopolysaccharidosis type | Morquio syndrome | Galactose 6-sulphase |
| IVA | | |
| Mucopolysaccharidosis type | Morquio syndrome | β-galactosidase |
| IVB | | |
| Mucopolysaccharidosis type VI | Maroteaux-Lamy | N-Acetylgalactosamine 4- |
| | syndrome | sulphatase |
| Mucopolysaccharidosis type VII | Sly syndrome | β-Glucuronidase |
| Mucopolysaccharidosis type IX | | hyaluronoglucosaminidase-I |
| Multiple sulphatase deficiency | | Multiple sulphatases |
| Neuronal Ceroid Lipofuscinosis, | Batten disease | Palmitoyl protein thioesterase |
| CLN1 | | |
| Neuronal Ceroid Lipofuscinosis, | Batten disease | Tripeptidyl peptidase I |
| CLN2 | | |
| Neuronal Ceroid Lipofuscinosis, | Vogt-Spielmeyer disease | Unknown |
| CLN3 | | |
| Neuronal Ceroid Lipofuscinosis, | Batten disease | Unknown |
| CLN5 | | |
| Neuronal Ceroid Lipofuscinosis, | Northern Epilepsy | Unknown |
| CLN8 | | |
| Niemann-Pick disease types | Niemaun-Pick disease | Acid sphyngomyelinase |
| A/B | | |
| Niemaun-Pick disease type C1 | Niemann-Pick disease | Cholesterol trafficking |
| Niemann-Pick disease type C2 | Niemann-Pick disease | Cholesterol trafficking |
| Pycnodysostosis | | Cathepsin K |
| Schindler disease types I/II | Schindler disease | α-Galactosidase B |
| Sialic acid storage disease | Sialuria, Salla disease | Sialic acid transporter |

The term “storage associated compound” use herein encompasses lipid containing primary storage material that accumulates in lysosomes of cells of the individual with the LSD concerned. The term storage associated compound also encompasses, lipid containing secondary material such as metabolites or catabolite of the primary storage material. The term storage associated material also encompasses lipid containing compounds the concentration of which alters as a consequence of the LSD such as might accumulates as a result of the proliferation of the membrane mass in the cells, or other secondary metabolic compounds that might for example decrease in level as a result of influence exerted by the increasing build up of primary storage material. The term is not intended to encompass the presence or absence of, for example, surface markers, specialised proteins such as enzymes or the like.

The estimated levels might refer directly to the principal storage compound and important candidates are secondary metabolites where these are lipid containing.

In certain forms of the invention the storage compounds might be very wide. They might include lipids and lipid containing macromolecules. The storage associated compounds might thus be selected from the group of compounds consisting of phospholipids and glycoconjugates

In forms where glycoconjugates are contemplated they might include glycolipids and lipopolysaccharides.

Glycolipids might be selected from the group comprising glycerolipids, glycoposhatidylinositols, glycosphingolipids. The glycosphingolipids might be selected from the group comprising neutral or acidic glycosphingolipids, monoglycosylceramides, or diosylcermaides, gangliosides, glycuronoglycosphingolipids, sulfatoglycosphingolipids, phosphoglycosphingolipids, phosphonoglycosphingolipids, sialoglycosphingolipids, uronoglycosphingolipids, sulfoglycosphingolipids, phosphoglycosphingolipids. Also contemplated may be sphinoglipids (including ceramide, glucosylceramide, trihexosylceramide), and globosides (including tetrahexosylceramides).

The phospholipid useful for the present invention is not intended to be limited. Phospholipids encompassed by the invention might be characterised by their head groups which might be selected from, but not limited to, the group consisting of phosphatidyl serine, phosphatidylinositol, phosphatidyl ethanolamine and sphingomyelin phosphatidyl glycerol, phosphatidyl serine, phosphatidyl inositol, phosophatidyl ethanolamine, cerebroside or a ganglioside.

The phospholipids might be characterised by the fatty acids which might be selected from, but not limited to, the group consisting of 1-palmitoyl-2-oleoyl-, 1-palmitoyl-2-linoleoyl-, 1-palmitoyl-2-arachadonyl-, 1-palmitoyl-2-docosahexanoyl. However other fatty acyl groups might also be chosen and could be selected from those having acyl chains of about 12 to about 18 carbon atoms. These tail group will be understood to be combined with any one of the head groups of the immediately preceding paragraph.

The method of measuring the presence and relative levels of storage associated compounds is not important to the general approach of the invention, and might be selected from any convenient method. Such methods might include electrophoresis, chromatography, Gas chromatography, HPLC (High pressure Liquid Chromatography), Nuclear Magnetic resonance analysis, gas chromatography-mass spectrometry (GC-MS), GC linked to Fourier-transform infrared spectroscopy (FTIR), and silver ion and reversed-phase high-performance liquid chromatography (HPLC) as wells as mass spectrometry.

As the complex relationships between stored substrates and pathology in LSD become clearer there is an obvious advantage of providing for faster and more accurate methods to characterise and quantify these stored substrates. That is particularly the case where the storage associated compounds needs to be measured in complex biological samples such as urine, plasma, and blood. To that end it is preferred to use mass spectrometry. The type of mass spectrometry method selected from the group consisting of ionising mass spectrometry, quadrupole mass spectrometry, ion trap mass spectrometry, time-of-flight mass spectrometry and tandem mass spectrometry, and electrospray ionization (ESI), the later being considered advantageous.

Particularly advantageous is electrospray ionisation-tandem mass spectrometry (ESI-MSMS). The advent of electrospray ionisation-tandem mass spectrometry (ESI-MSMS) has made possible the simultaneous determination of large numbers of analytes from complex mixtures. For newborn screening, ESI MSMS enables the concurrent determination of a wide range of amino acids and acyl carnitines as their butyl esters. This technology is used to screen for over twenty different genetic disorders, including the amino acidopathies and the fatty acid oxidation defects (6,7). ESI-MSMS has been used effectively to investigate stored substrates in a number of LSD and has great potential in the field of this invention.

It has become evident that the levels of a single storage associated compound are not sufficient to give a clear distinction between varying degrees of exposure of an individual to the effects of an LSD. A comparison between at least two markers is required for a quantitative relationship to emerge. The relationship might be additive so that both storage associated compounds increase in the levels in which they are found where the condition is present, and a comparison is made to an internal control. Preferably in devising the method where at least two compounds are selected one from a first group that increase and a second from a second group that decreases in levels. The values are combined mathematically to arrive at an index number. The relative levels of those two compounds leads to an amplification of the differences between LSD affected individuals and the control population. As indicated earlier the severity of the condition and the index number have a direct correlation. Conversely therefore the value of the index number can be compared to a standard to provide a indication of the level of severity of the condition.

It has been found that a difference in index number between individuals that are positive or negative for an LSD condition by use of such combination can be made statistically significant provided an appropriate combination of storage associated compounds is used.

Samples for analysis can be obtained from any organ, tissue, fluid or other biological sample comprising lysosomes or their component storage associated compounds. A preferred sample is whole blood and products derived therefrom, such as plasma and serum. Blood samples may conveniently be obtained from blood-spot taken from, for example, a Guthrie card.

Other sources of tissue for example are skin, hair, urine, oral fluids, semen, faeces, sweat, milk, amniotic fluid, liver, heart, muscle, kidney, brain and other body organs. Tissue samples comprising whole cells are typically lysed to release the storage associated compounds.

The present method may be used as an early test and thus samples can be obtained from embryos, foetuses, neonatals, young infants.

Most preferably the sample is one readily obtainable such as a blood samples. Whilst obtaining these is invasive they are routinely taken and generally therefore are not inconvenient. It may be preferred to have a non-invasive sample such as urine, oral fluid or buccal smear. There are however variations in the value of certain metabolites in urine resulting from variation in salt content, such as oxalic acid, and in saliva there is variation in the capacity of individuals to secrete certain compounds.

It is found that with Gaucher patients that the LSD index number was not only a qualitative measure but also a qualitative measure being indicative of the severity of the condition. Thus the status of the LSD being assessed may not only be to ascertain the presence or absence but might also include the degree of severity. The status might also include subclinical levels of the condition that relate to levels achieved before onset of physical manifestations become apparent. This invention will be understood to have application to monitoring treatment, for example with individuals undergoing enzyme or other therapy.

Thus individuals with Gaucher disease that undergo enzyme replacement therapy have a index number that is considerably lower than untreated individuals. It is also desirable that the doses of active enzyme delivered to sufferers is kept to a minimum if only from a cost perspective but perhaps also from a perspective of minimising any adverse affects of the treatment. Thus the present method may be used particularly for monitoring treatment of an LSD sufferer, or for ascertaining initially and perhaps from time to time as the sufferer ages the most appropriate dose of active to be delivered, and thus individuals diagnosed may be tested from time to time to ascertain the severity of the condition. It is less critical that the test discriminates quite as distinctly from non-LSD sufferers because all that is required is that the relative level of severity can be quantified. Thus whilst it may be necessary to screen using indicators of the concentration of three or more lipid containing compounds to distinguish over non-LSD sufferers the monitoring may only require indicators of two lipid containing compounds and may be carried out using less precise measuring methods.

The invention has particular applicability to human conditions. Certain mammals are also susceptible to LSD and the invention may be useful where the individual is a non-human mammal. For examples α-mannosidoses is relatively common in certain breeds of cattle and screening may be a useful stock management tool.

### Example 1

### Monitoring of Therapy for Gaucher Disease

This report provides a detailed analysis of the initial trial of our developed methodology to monitor enzyme replacement therapy (ERT) in Gaucher disease using dried blood spots.

Patient samples: Dried blood spots have been collected from five Australian Gaucher patients receiving ERT for the past two years (12 samples). Sixteen dried blood spots have been collected from patients not receiving ERT, from referrals to the National Referral Laboratory for Lysosomal, Peroxisomal and Related Diseases (which is based in our parent Department). In addition, through collaboration with Dr Eugene Mengel (Germany), we have obtained 39 samples from German Gaucher disease patients receiving ERT, and three samples from untreated patients. Dried blood spots have been collected from 10 unaffected adults as control samples. Total sample numbers are as shown in Table 1.

Sample preparation: From each Guthrie card sample a 3 mm dried blood spot was punched and the lipids were eluted (16 h) with 200 μL of isopropanol containing 200 nmol of each internal standard; Cer C17:0, GC(d3)C16:0, LC(d3)C16:0, PC C14:0. The blood spots were removed and the isopropanol dried under a stream of nitrogen. Lipids were redissolved in 100 μL of methanol containing 10 mM NH₄COOH for analysis by mass spectrometry.

Mass spectrometry: Mass spectrometric analysis of lipids was performed using a PE Sciex API 3000 triple-quadrupole mass spectrometer with a turbo-ionspray source and Analyst data system (PE Sciex, Concord, Ontario, Canada). Samples (20 μL) were injected into the electrospray source with a Gilson 233 autosampler using a carrying solvent of methanol at a flow rate of 80 μL/minute. For all analytes nitrogen was used as the collision gas at a pressure 2×10⁻⁵ Torr. Lipids were analysed in +ve ion mode. Determination of lipids was performed using the multiple-reaction monitoring (MRM) mode. Seventeen different glycosphingolipid and ceramide species were monitored using the ion pairs shown in Table 2. Each ion pair was monitored for 100 milliseconds and the measurements were repeated and averaged over the injection period. Determination of lipids was achieved by relating the peak height of each lipid ion signal to the peak height of the signal from the corresponding internal standard (Table 2).

### Results

To determine which analytes were potentially useful markers for monitoring Gaucher disease, the patients were grouped into control (group 1, n=10), Gaucher patients receiving ERT (group 2, n=51), and untreated Gaucher patients (group 3, n=19). Mann-Whitney U values were then calculated for each analyte to determine the difference between the control and untreated patients, control and treated patients, and treated and untreated patients. These results are shown in Table 3.

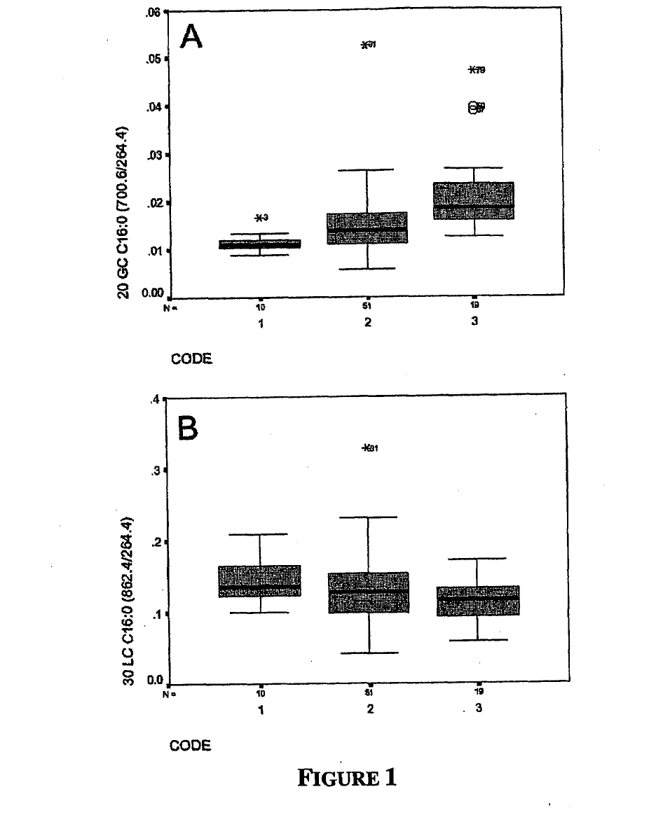

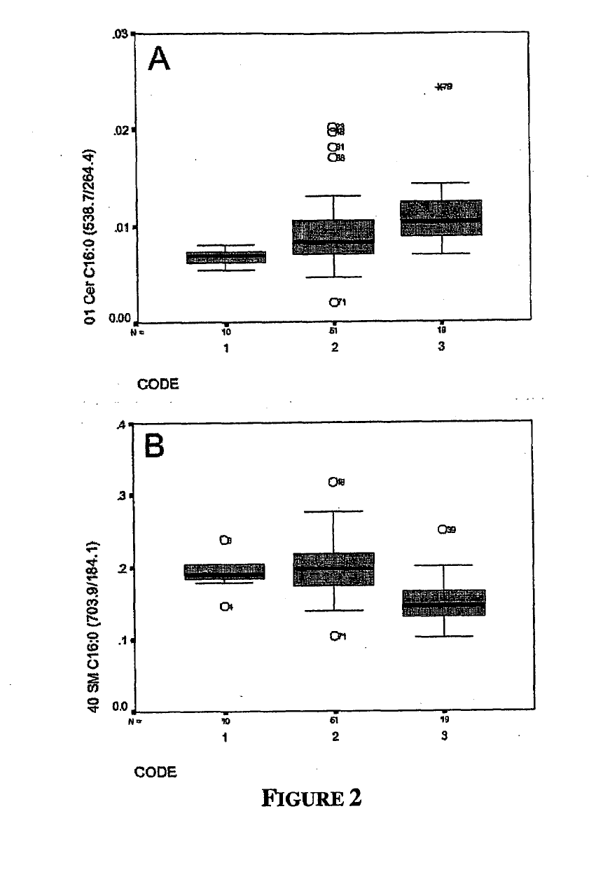

We observed that, in addition to the expected elevation of glucosylceramide (GC) in the untreated Gaucher patients compared to controls, there were significant differences in the level of ceramide C16:0 and the sphingomyelin species C16:0, C22:0 and C24:0 (all significant to the 0.01 level). The same markers also showed a significant difference between treated and untreated Gaucher patients. Of the lactosylceramide and trihexosylceramide species only the C16:0 species showed a significant difference between control and untreated patients (significant to the 0.05 level). The box plots of each C16:0 species of ceramide, GC, LC and sphingomyelin (FIGS. 1 and 2) show that whilst there is an observed increase in the level of ceramide and GC in untreated patients, the levels of sphingomyelin and LC are decreased. In addition, the level of these analytes in the treated patients generally fell between the control and untreated patients. In each case ERT has partially normalised the lipid levels, although not in all patients.

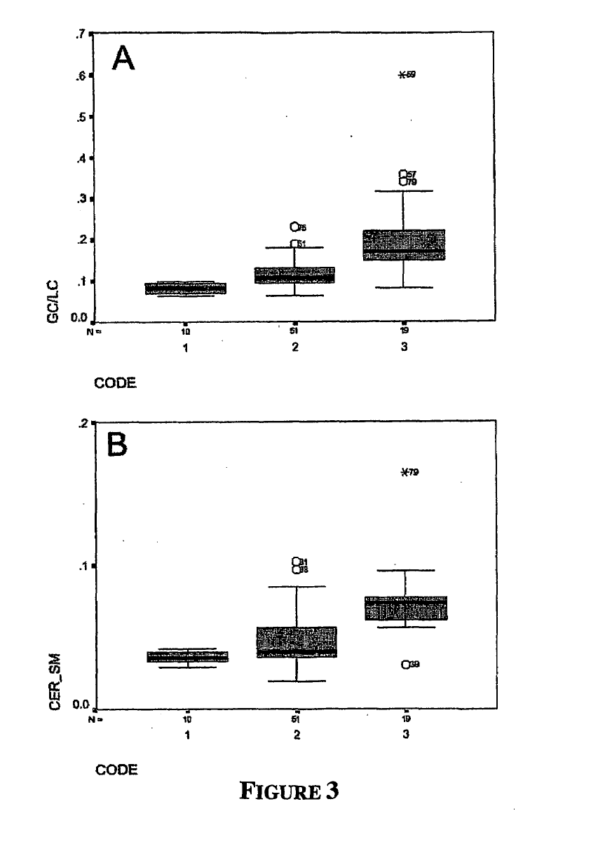

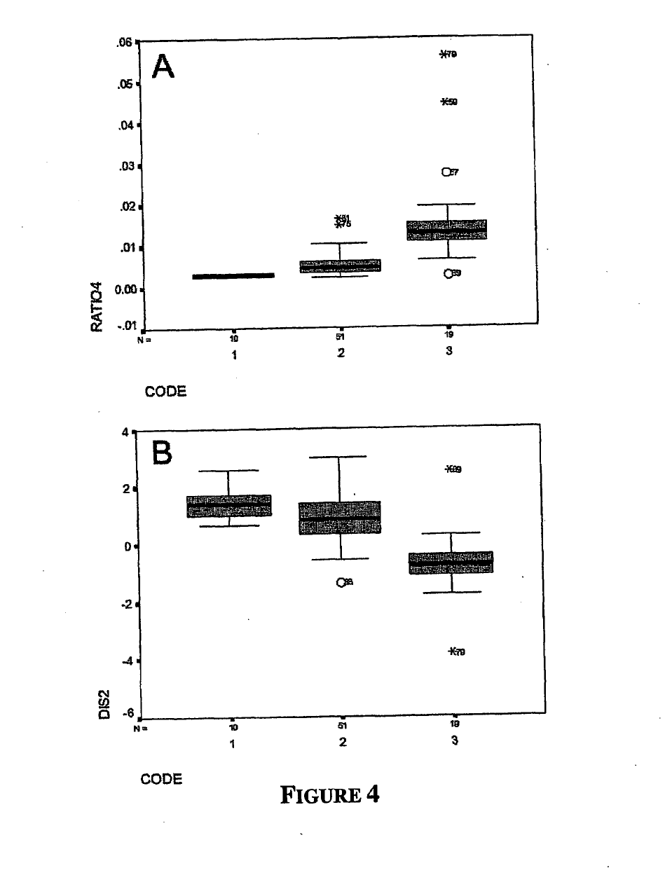

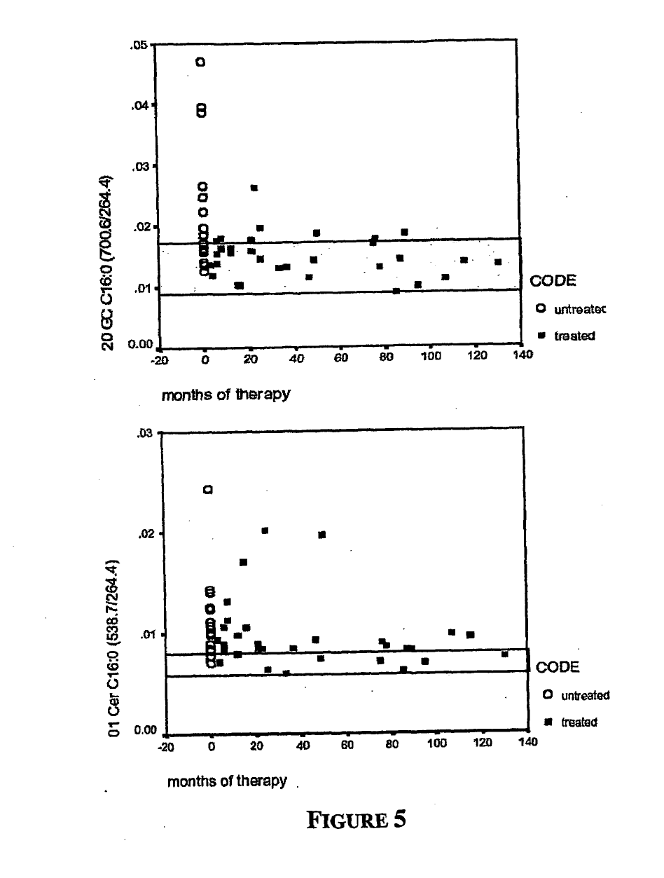

Although the observed differences between control and untreated patients are significant there is still considerable overlap between the two populations. This is due, at least in part, to the range of lipid levels in the control and patient groups. To improve the discrimination of the markers we investigated the use of multiple markers by plotting ratios of GC/LC or ceramide/sphingomyelin (FIG. 3). As GC and ceramide levels increase in Gaucher patients, while the LC and sphingomyelin decrease, these ratios provided improved discrimination between groups. Utilising all four analytes in a combined ratio (Ratio4=(GC C16:0*Cer C16:0)/(LC C16:0*SM C16:0) further improved the discrimination. Similarly discriminate analysis using the four C16:0 species resulted in a function (Dis2=(−195*Cer C16:0)−(29.8*GC C16:0)+(12.3*LC C16:0)+(16.9*SM C16:0)−1.91)) with improved discrimination. (FIG. 4 and Table 3).

Clearly, the use of multiple analytes or lipid profiles provides a better representation of lipid metabolism in control and Gaucher patients. The ratio4 and discriminate function (Dis2) plotted in FIG. 4 show almost total separation of the control and untreated Gaucher patient groups, with the patient group being partially normalised (although many treated patients were not completely normalised).

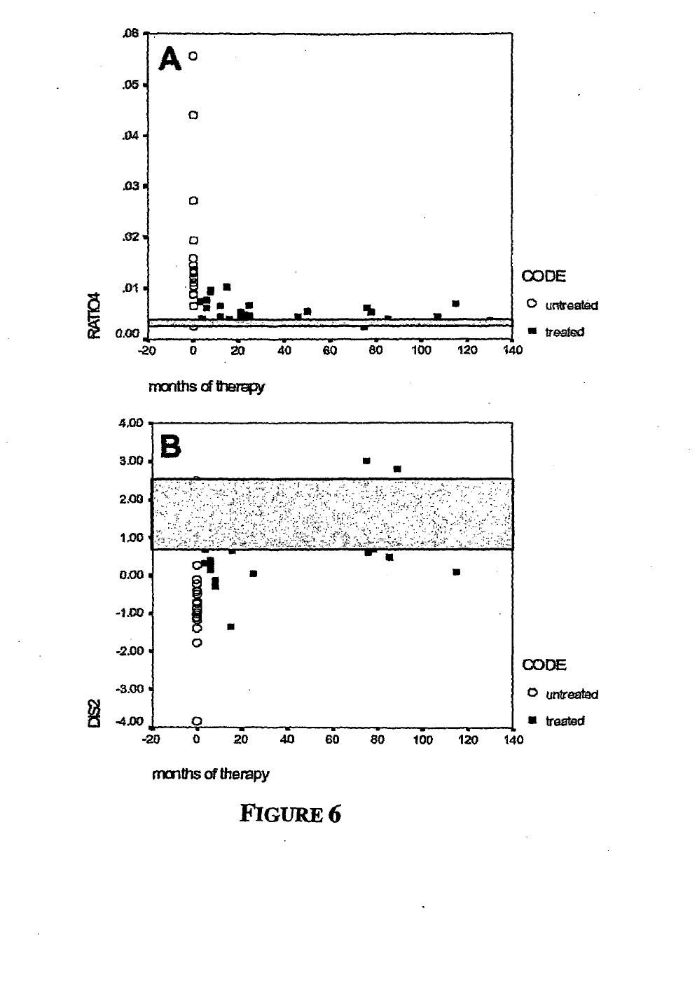

We investigated what effect time on therapy had on a number of the same analytes and analyte ratios (FIGS. 5 and 6). The GC and ceramide levels showed a trend towards normalisation with increasing time on therapy, however in a number of patients the ceramide level did not reach the normal range even at 80-120 months on therapy. The use of the ratio and the discriminate function (FIG. 6) showed similar results with some patients normalising with time but others outside the normal range even after 80-120 months of therapy.

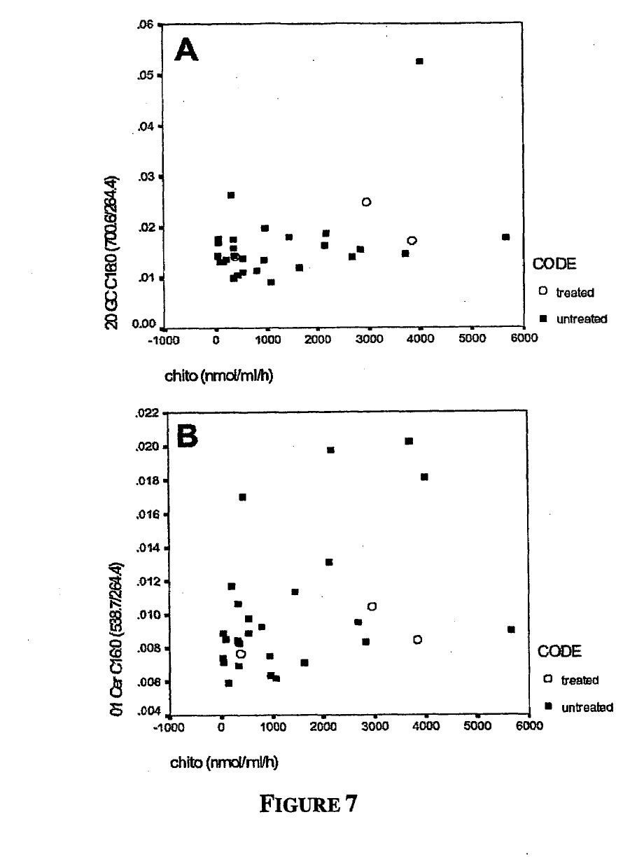

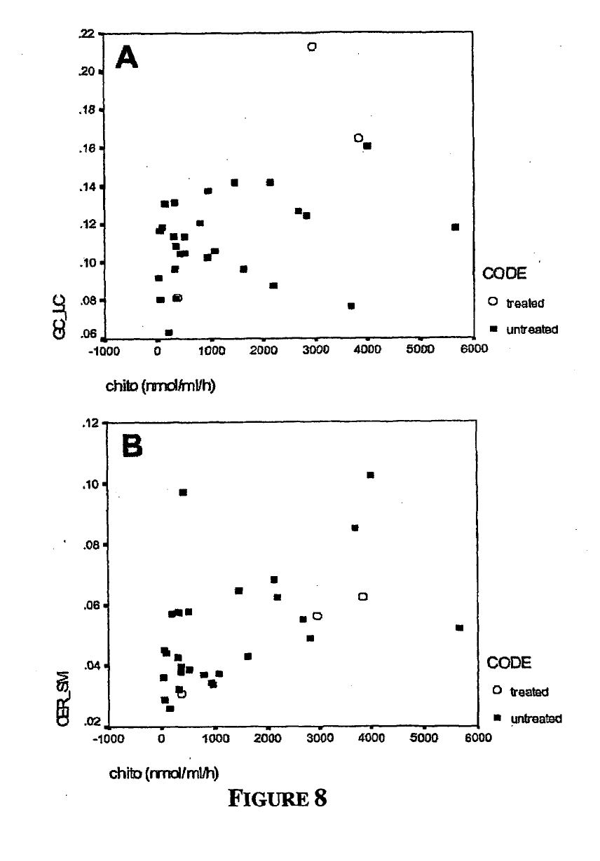

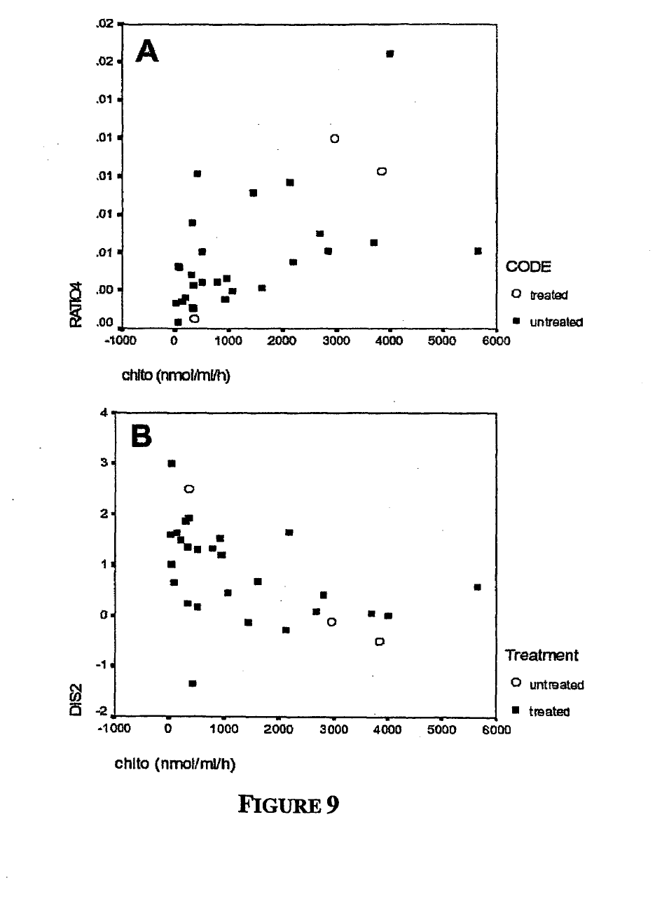

The relationship between the glycolipid markers and ratios, and the macrophage activation marker chitotriosidase is shown in FIGS. 7-9; a significant correlation is observed for the ceramide and GC as well as for the ratios GC/LC, ceramide/sphingomyelin and ratio4, and for the discriminate function Table 4 shows the Pearson correlation coefficients for these markers with chitotriosidase and other markers that have been used to monitor ERT in Gaucher disease including angiotensin converting enzyme, lysozyme and acid phosphatase. In general the correlations are stronger between these markers and the lipid ratios, rather than single lipid species.

### Discussion

In this study we have provided evidence that the primary storage substrate GC is a useful marker for monitoring Gaucher disease. We observe an increased level of GC in dried blood spots from untreated patients compared to controls and a normalisation of GC levels after ERT. This is an expected outcome, based on the known biochemistry of Gaucher disease. Somewhat less expected is the elevation in ceramide and the decrease in LC and sphingomyelin. We have previously reported that LC is decreased in the plasma of Gaucher patients and that the ratio of GC/LC provides a better discrimination of Gaucher patients from controls than the GC levels on their own (Whitfield et al 2002). In these preliminary studies we have identified that other lipids are also affected, particularly ceramide and sphingomyelin. We have also shown that using a combination of these analytes with the GC and LC levels, as either a ratio or a discriminate function, provides greater discrimination and potentially a better mechanism for monitoring ERT in Gaucher disease than the use of individual analytes. The ratio4 and the discriminate function Dis2 are based on the limited numbers in this study and require further refinement, however they provide an initial demonstration of the power of metabolic profiling for the characterisation of patients and the monitoring of therapy in Gaucher disease.

Our hypothesis is that the level of GC within a normal population will fall within a specified range, which is affected by many metabolic parameters affecting the biosynthesis and degradation of GC. In the Gaucher disease population this range will be altered as a result of the metabolic defect; however, those Gaucher patients with the lower GC levels are likely to overlap with unaffected controls with the higher GC levels. This results in uncertainties in the interpretation of GC levels in isolation with regard to Gaucher disease status, and difficulties in determining normalisation following ERT.

However, with a metabolic profile (multiple analytes) the breadth of the normal range will be decreased, as each of these analytes is related to the others by the metabolic pathways that exist. Consequently, the power to discriminate normal from Gaucher disease is increased and the ability to measure the normalisation of patients on treatment is improved.

TABLE 1
Patient and control samples included in this trial

| | | Age | |
|---|---|---|---|
| Patient group | Number | Median (range) | Comment |
| Control | 10 | 38 (23-56) | |
| Treated Gaucher | 51 | 23 (2-72) | All type 1 |
| Untreated Gaucher | 19 | 24 (1-36) | 2 type 3, 14 type 1, |
| | | | 3 unknown |

TABLE 2
Lipid analytes used for Gaucher Monitoring

| | | MRM ion |
|---|---|---|
| Lipid analytes^{a} | Internal standard | pairs (m/z) |
| Cer C16:0 | Cer C17:0 | 538.7/264.4 |
| Cer C24:0 | Cer C17:0 | 650.7/264.4 |
| Cer C24:1 | Cer C17:0 | 648.7/264.4 |
| Cer C17:0 (internal standard) | | 552.7/264.4 |
| GC C16:0 | GC(d3)C16:0 | 700.6/264.4 |
| GC C22:0 | GC(d3)C16:0 | 784.7/264.4 |
| GC C24:0 | GC(d3)C16:0 | 812.7/264.4 |
| GC C24:1 | GC(d3)C16:0 | 810.8/264.4 |
| GC(d3)C16:0 (internal standard) | | 703.8/264.4 |
| LC C16:0 | LC(d3)C16:0 | 862.4/264.4 |
| LC C24:0 | LC(d3)C16:0 | 974.8/264.4 |
| LC C24:1 | LC(d3)C16:0 | 972.8/264.4 |
| CTH C16:0 | LC(d3)C16:0 | 1024.1/264.4 |
| CTH C22:0 | LC(d3)C16:0 | 1108.1/264.4 |
| CTH C24:0 | LC(d3)C16:0 | 1136.6/264.4 |
| CTH C24:1 | LC(d3)C16:0 | 1134.1/264.4 |
| LC(d3)C16:0 (internal standard) | | 865.6/264.4 |
| SM C16:0 | PC C14:0 | 703.9/184.1 |
| SM C22:0 | PC C14:0 | 787.8/184.1 |
| SM C24:0 | PC C14:0 | 815.8/184.1 |
| PC C14:0 (internal standard) | | 678.5/184.1 |

^{a}Cer = ceramide, GC = glucosylceramide, LC = lactosylceramide, CTH = ceramide trihoxoside, SM = sphingomyelin, PC = phosphatidyicholine

TABLE 3
Mann-Whitney U values for lipid analytes and ratios of analytes,
between controls^{a}, untreated Gaucher patients^{b} and
Gaucher patients treated with enzyme replacement therapy^{c}.

| | Control vs | | Control vs | | Untreated vs | |
|---|---|---|---|---|---|---|
| | Untreated | | Treated | | Treated | |
| Analyte | M-W U^{d} | Sig.^{e} | M-W U^{d} | Sig.^{e} | M-W U^{d} | Sig.^{e} |
| Cer C16:0 | 6 | 0.000 | 111 | 0.004 | 300 | 0.009 |
| Cer C24:1 | 73 | 0.313 | 215 | 0.342 | 478 | 0.740 |
| Cer C24:0 | 56 | 0.070 | 174 | 0.087 | 447 | 0.466 |
| GC C16:0 | 9 | 0.000 | 139 | 0.017 | 240 | 0.001 |
| GC C22:0 | 26 | 0.002 | 142 | 0.021 | 307 | 0.012 |
| GC C24:1 | 19 | 0.000 | 101 | 0.002 | 271 | 0.003 |
| GC C24:0 | 28 | 0.002 | 149 | 0.029 | 319 | 0.018 |
| LC C16:0 | 49 | 0.033 | 222 | 0.419 | 358 | 0.063 |
| LC C24:0 | 75 | 0.359 | 183 | 0.121 | 450 | 0.490 |
| LC C24:1 | 62 | 0.130 | 228 | 0.481 | 434 | 0.375 |
| CTH C16:0 | 52 | 0.046 | 149 | 0.028 | 392 | 0.152 |
| CTH C22:0 | 83 | 0.582 | 127 | 0.009 | 166 | 0.000 |
| CTH C24:1 | 88 | 0.748 | 103 | 0.002 | 189 | 0.000 |
| CTH C24:0 | 54 | 0.060 | 179 | 0.104 | 472 | 0.687 |
| SM C16:0 | 31 | 0.003 | 239 | 0.618 | 149 | 0.000 |
| SM C22:0 | 29 | 0.002 | 203 | 0.240 | 187 | 0.000 |
| SM C24:0 | 33 | 0.004 | 219 | 0.382 | 219 | 0.000 |
| GC_LC | 6 | 0.000 | 80 | 0.001 | 169 | 0.000 |
| CER_SM | 9 | 0.000 | 150 | 0.031 | 138 | 0.000 |
| RATIO4^{f} | 7 | 0.000 | 64 | 0.000 | 96 | 0.000 |
| DIS2^{g} | 9 | 0.000 | 164 | 0.057 | 86 | 0.000 |

^{a}controls n = 10
^{b}untreated n = 19
^{c}treated n = 51
^{d}Mann-Whitney U values
^{e}significance (two-tailed)
^{f}Ratio4 = (GC C16:0*Cer C16:0)/(LC C16:0*SM C16:0)
^{g}Dis2 = (−195*Cer C16:0) − (29.8*GC C16:0) + (12.3*LC C16:0) + (16.9*SM C16:0) − 1.91

TABLE 4
Pearson Correlation coefficients between lipid markers and
other markers used in Gaucher disease.

| | months of | | chitotriosidase | | | | lysozyme | | acid | |
|---|---|---|---|---|---|---|---|---|---|---|
| | therapy^{b} | | (nmol/ml/h) | | ACE (U/l)^{e} | | (mg/l) | | phosphatase | |
| | PCC^{c} | | PCC | | PCC | | PCC | | PCC | |
| Analyte^{a} | N = 51 | Sig.^{d} | N = 30 | Sig. | N = 40 | Sig. | N = 38 | Sig. | N = 40 | Sig. |
| Cer C16:0 | −0.24 | 0.08 | 0.40 | 0.03 | 0.42 | 0.01 | 0.40 | 0.01 | 0.44 | 0.00 |
| GC C16:0 | −0.32 | 0.02 | 0.41 | 0.02 | 0.36 | 0.02 | 0.23 | 0.17 | 0.52 | 0.00 |
| LC C16:0 | 0.19 | 0.18 | 0.16 | 0.38 | 0.10 | 0.53 | 0.01 | 0.96 | 0.17 | 0.30 |
| CTH C16:0 | 0.00 | 1.00 | −0.10 | 0.60 | −0.03 | 0.83 | 0.34 | 0.04 | −0.01 | 0.95 |
| SM C16:0 | 0.51 | 0.00 | −0.29 | 0.13 | −0.24 | 0.13 | 0.04 | 0.82 | −0.23 | 0.15 |
| GC/LC | −0.35 | 0.01 | 0.42 | 0.02 | 0.41 | 0.01 | 0.23 | 0.17 | 0.50 | 0.00 |
| CER/SM | −0.47 | 0.00 | 0.52 | 0.00 | 0.50 | 0.00 | 0.35 | 0.03 | 0.53 | 0.00 |
| RATIO4 | −0.38 | 0.01 | 0.59 | 0.00 | 0.58 | 0.00 | 0.39 | 0.01 | 0.70 | 0.00 |
| DIS2 | 0.54 | 0.00 | −0.49 | 0.01 | −0.47 | 0.00 | −0.26 | 0.11 | −0.47 | 0.00 |

^{a}Cer = ceramide, GC = glucosylceramide, LC = lactosylceramide, CTH = ceramide trihexoside, SM = sphingomyelin, Ratio4 = (GC C16:0*Cer C16:0)/(LC C16:0*SM C16:0), Dis = (−195*Cer C16:0) − (29.8*GC C16:0) + (12.3*LC C16:0) + (16.9*SM C16:0) − 1.91
^{b}months on enzyme replacement therapy
^{c}PCC = Pearson correlation coefficient
^{d}Sig. = significance (two tailed)
^{c}ACE = angiotensin converting enzyme

### Example 2

### Identification of Fabry Hemizygous and Heterozygous Individuals Using Lipid Profiles

This report summarises the results of analyses performed on urine, plasma and dried blood spots from control, Fabry heterozygote and Fabry patient groups.

### Materials and Methods

Patient samples: Urine samples have been collected from 14 Fabry patients (two of whom had renal transplants), 13 Fabry heterozygotes (three of whom had reported clinical symptoms) and 20 unaffected controls. Plasma samples were retrieved from archival sources in the Department of Chemical Pathology and represented 29 Fabry patients, three Fabry heterozygotes and 10 control samples. Dried blood spots on filter paper (Guthrie cards) were collected from 13 Fabry patients, two Fabry heterozygotes and 10 control individuals.

Sample preparation and analysis: Urine, plasma and dried blood spot samples were prepared as described in Appendices I, II and III, and analysed for lipids by mass spectrometry.

Mass spectrometry: Mass spectrometric analysis of lipids was performed using a PE Sciex API 3000 triple-quadrupole mass spectrometer with a turbo-ionspray source and Analyst data system (PE Sciex, Concord, Ontario, Canada). Samples (20 μL) were injected into the electrospray source with a Gilson 233 autosampler using a carrying solvent of methanol at a flow rate of 80 μL/minute. For all analytes nitrogen was used as the collision gas at a pressure 2×10⁻⁵ Torr. Lipids were analysed in +ve ion mode. Lipid analysis was performed using the multiple-reaction monitoring (MRM) mode. Twenty-two different ceramide, glycosphingolipid and sphingomyelin species were monitored using the ion pairs shown in Table 5. In urine samples seven additional phosphatidylcholine species were also monitored (Table 5). Each ion pair was monitored for 100 milliseconds and the measurements were repeated and averaged over the injection period. Determination of lipids was achieved by relating the peak height of each lipid ion signal to the peak height of the signal from the corresponding internal standard (Table 5).

### Results

Analysis of Urine: Lipid profiling of the urine samples from control, Fabry and Fabry heterozygotes (Fabry het) has been performed. In all, 29 lipid species were determined including ceramide (Cer), glucosylceramide (GC), lactosylceramide (LC), trihexosylceramide (CTH), sphingomyelin (SM) and phosphatidylcholine (PC) species. Appropriate internal standards were used that provide absolute quantification of these species (expressed as nmol/L urine). PC was included as a general marker of urinary sediment, as we had previously observed this to be a more useful correction factor for the determination of urinary lipids than creatinine. This relates to the urinary lipids being derived from epithelial cells of the kidneys, bladder and urinary tract rather than filtered through the kidneys; PC is a major lipid constituent of these cells and so is a useful measure of the level of urinary sediment.

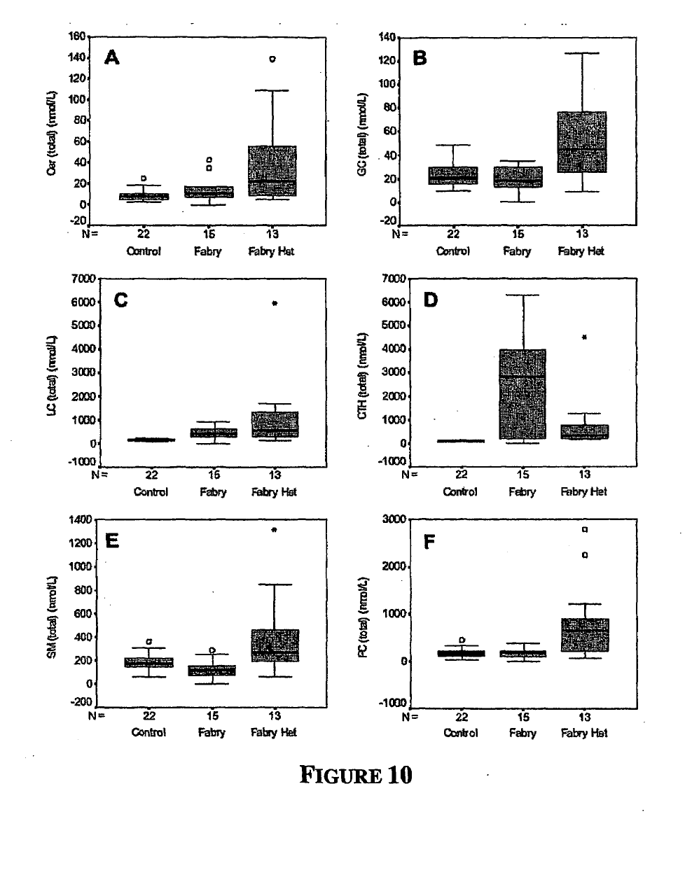

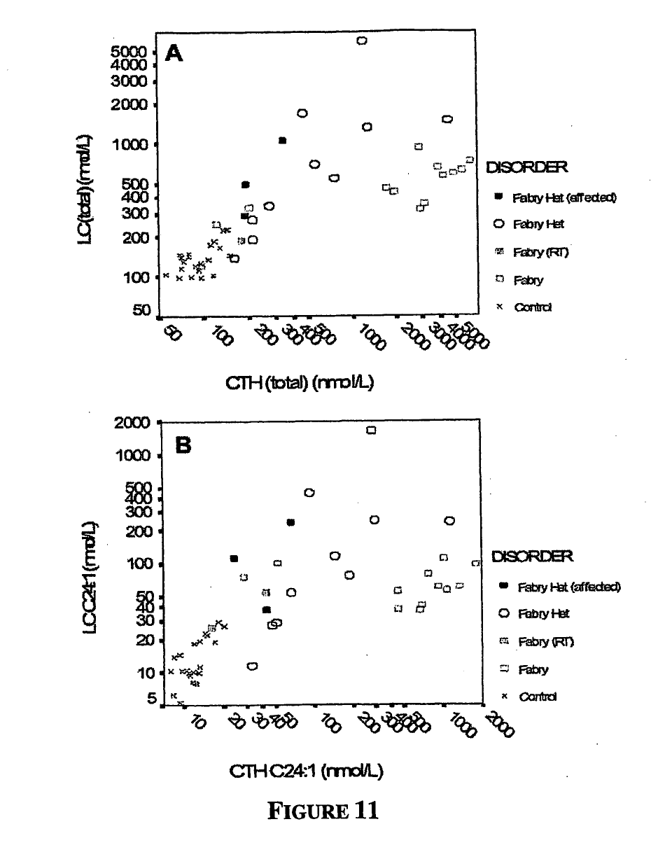

An initial statistical analysis was performed on the data as expressed as nmol/L urine. Mann-Whitney U values were determined to compare the control group with the Fabry and Fabry het groups (Table 6). Examination of these results shows that many of the lipid analytes are significantly different in the patient groups compared to the control groups. The Fabry and Fabry het groups show a significant difference to the control group in many lipid species, including Cer, LC, CTH and SM. Interestingly, the level of PC in the Fabry het group is significantly elevated above the control population, while no significant difference between the control and Fabry groups is observed. Examination of the range of analytes for each group (FIG. 10) shows that for all analytes except CTH, the Fabry het group is elevated above the control and Fabry groups. The observed elevation of these lipids suggests that the Fabry het group has elevated urinary sediment compared to the control and Fabry groups.

The scatter plot of LC (total) versus CTH (total) (FIG. 11A) shows that the use of lipid levels (nmol/L urine) can differentiate between Fabry patients and the control group, although there is some overlap between both Fabry and Fabry het and the control group. The use of the specific lipid species LC C24:1 and CTH C24:1 (FIG. 11B) improved this discrimination, although some overlap still exists. A concern with these results is that the differentiation of the Fabry het group from the control group reflects the elevated urinary sediment rather than an altered lipid profile. Consequently, individuals who are not affected by Fabry disease but who have an elevation in urinary sediment would be falsely identified as a Fabry het using this type of analysis.

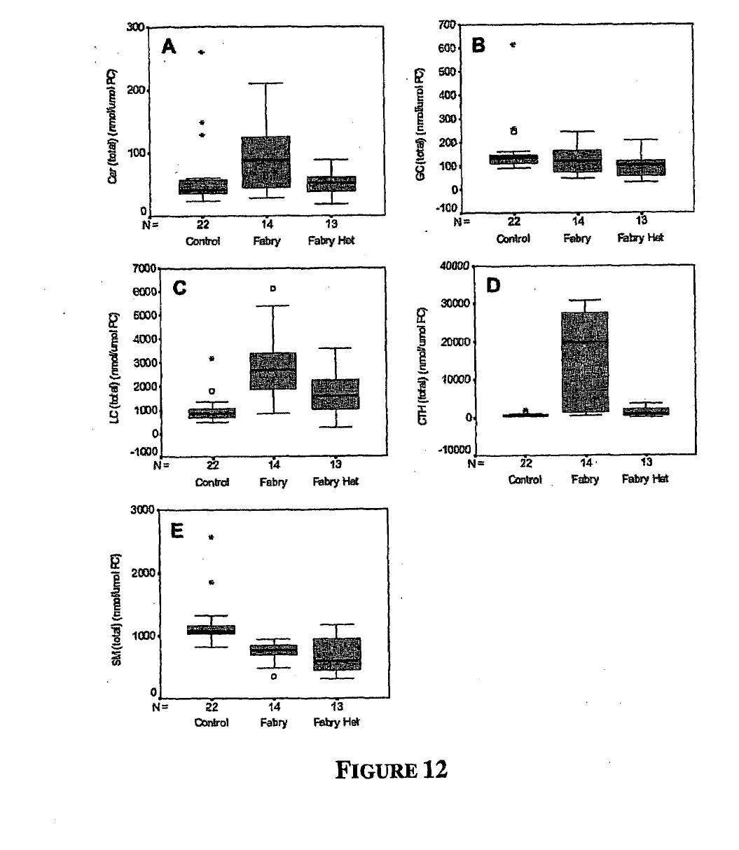

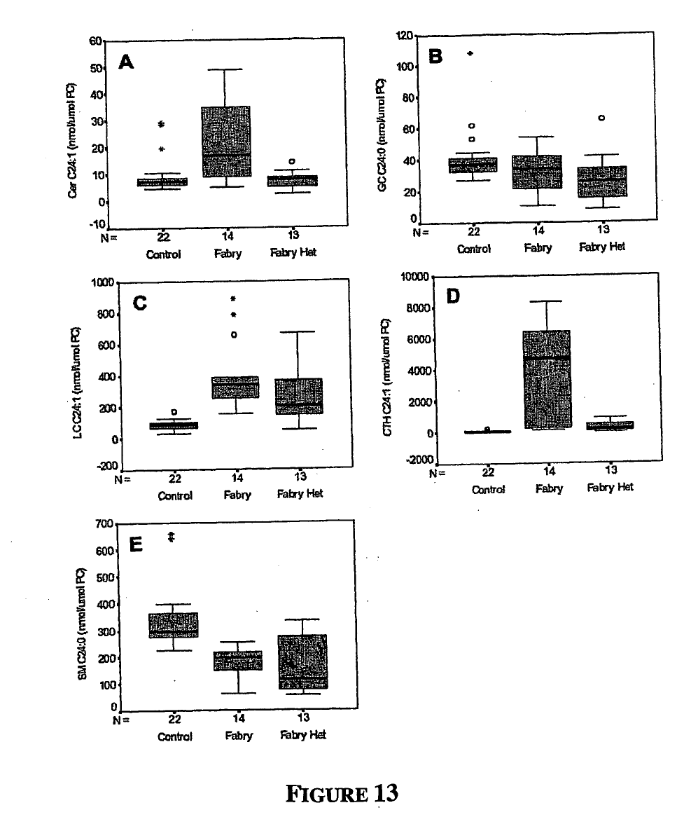

To address this, correction was made for each lipid analyte value for the level of PC (total) in each sample; statistical analysis on these data was performed. Table 7 shows the Mann-Whitney U values for each patient group compared to the control group. The corrected data also show multiple analytes to be significantly different between the control and patient groups. The box plots in FIG. 12 show the range of each analyte group (corrected for PC). These plots show that the Fabry group has elevated CTH, LC and Cer and decreased SM, whereas the Fabry het group now shows an elevation in CTH and a much lower elevation in LC and Cer. Interestingly, the Fabry het group shows a larger decrease in the SM than the Fabry group. This may relate to a sex difference, although no difference was seen between the males and females in the control group. Larger sample numbers will be required to confirm this.

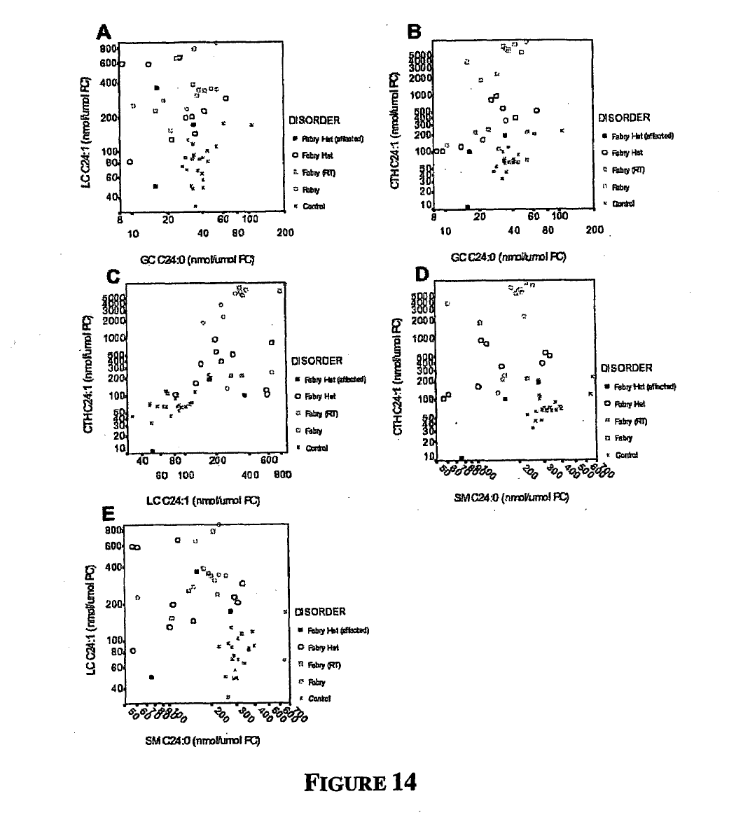

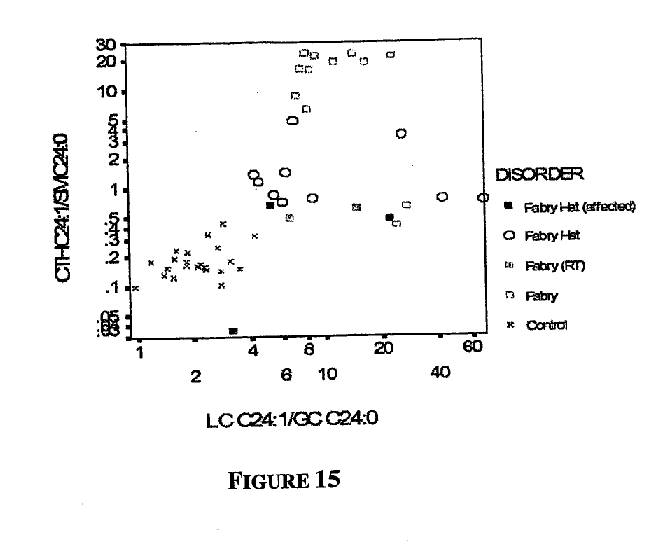

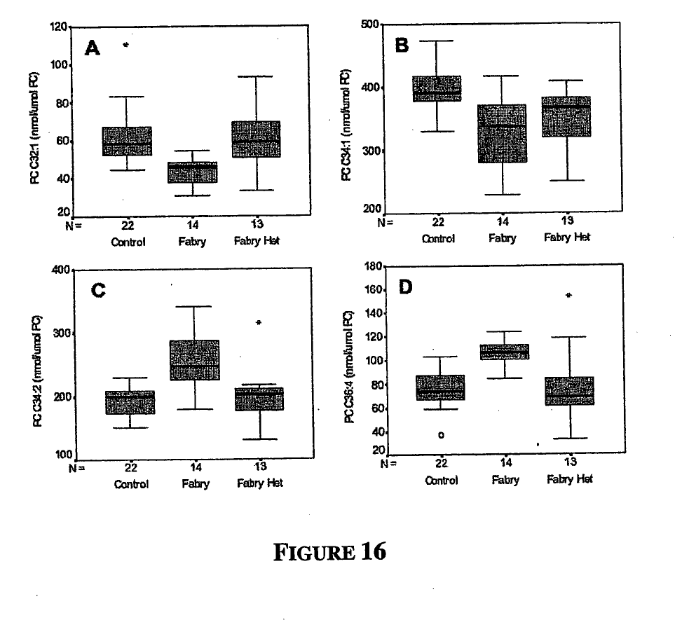

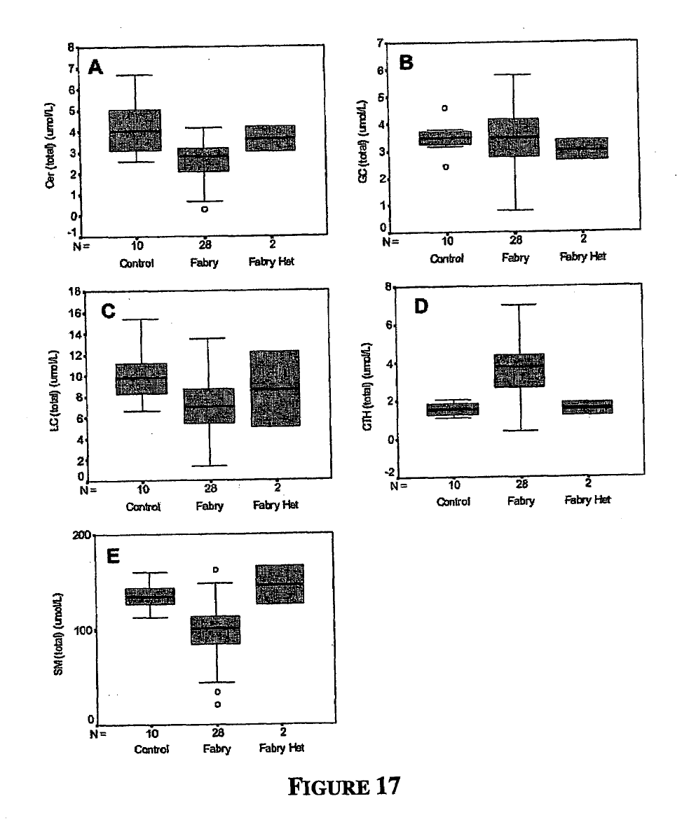

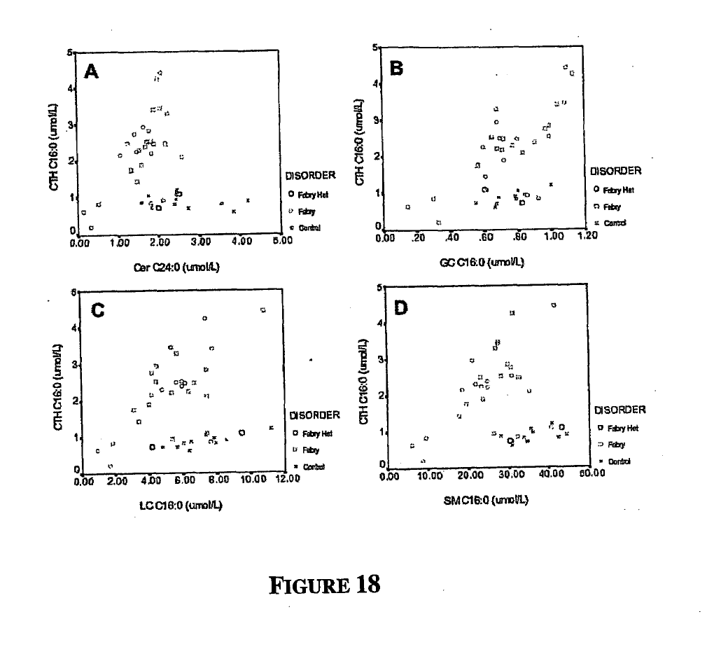

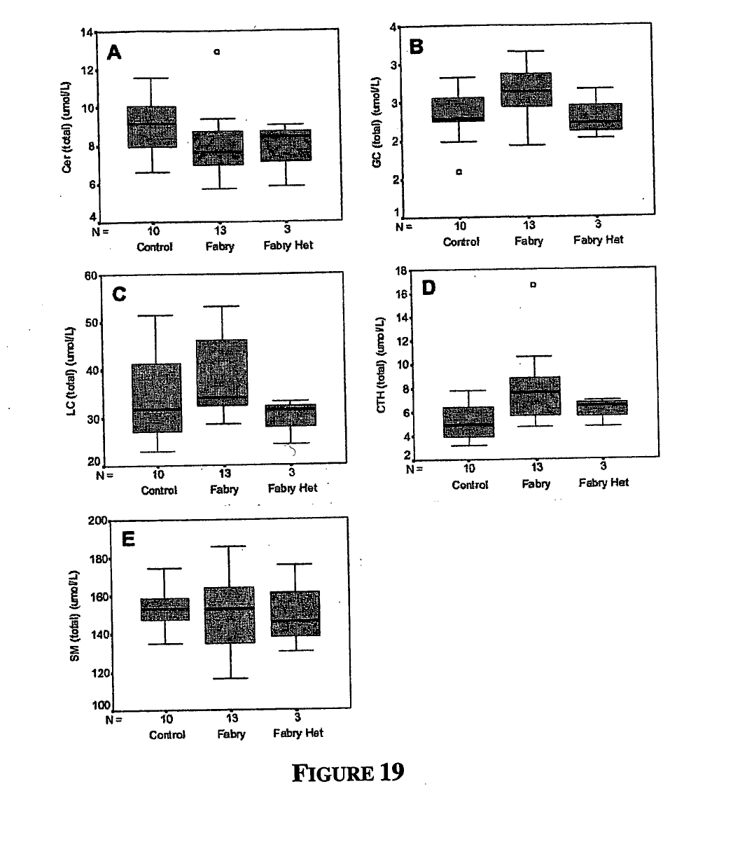

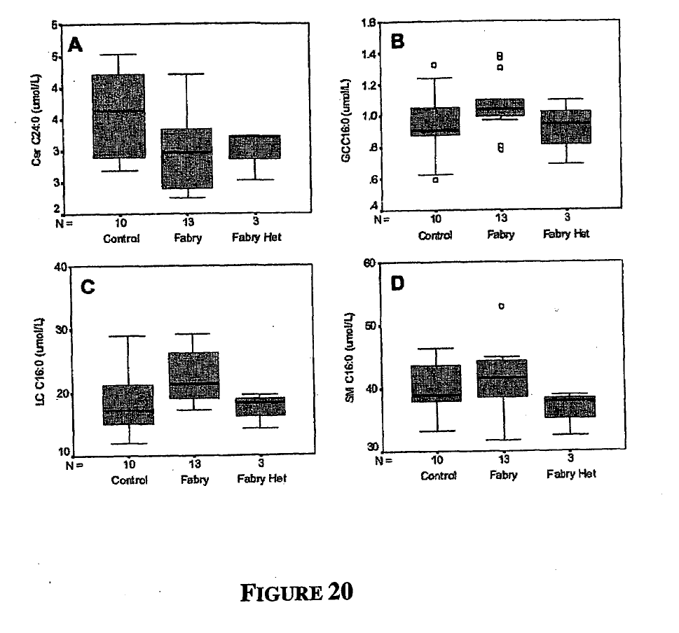

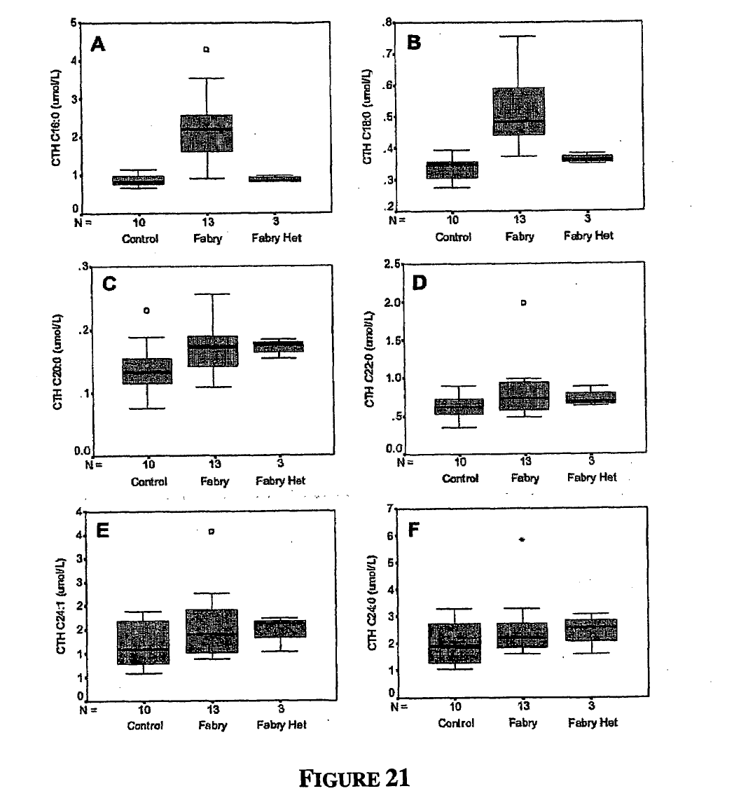

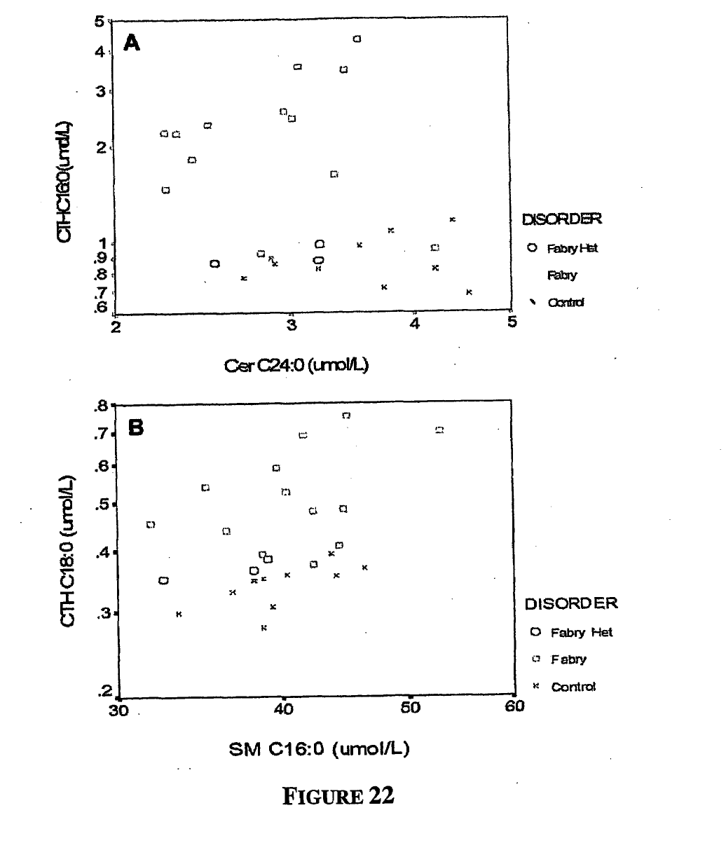

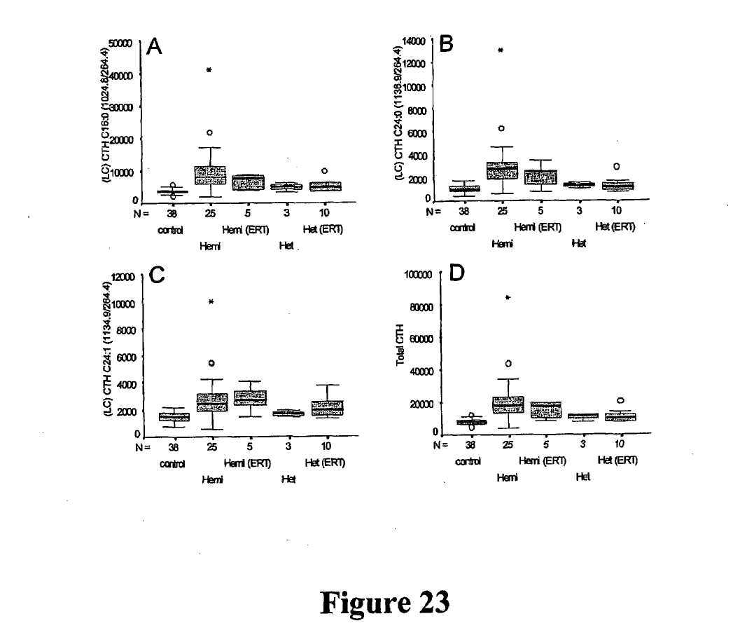

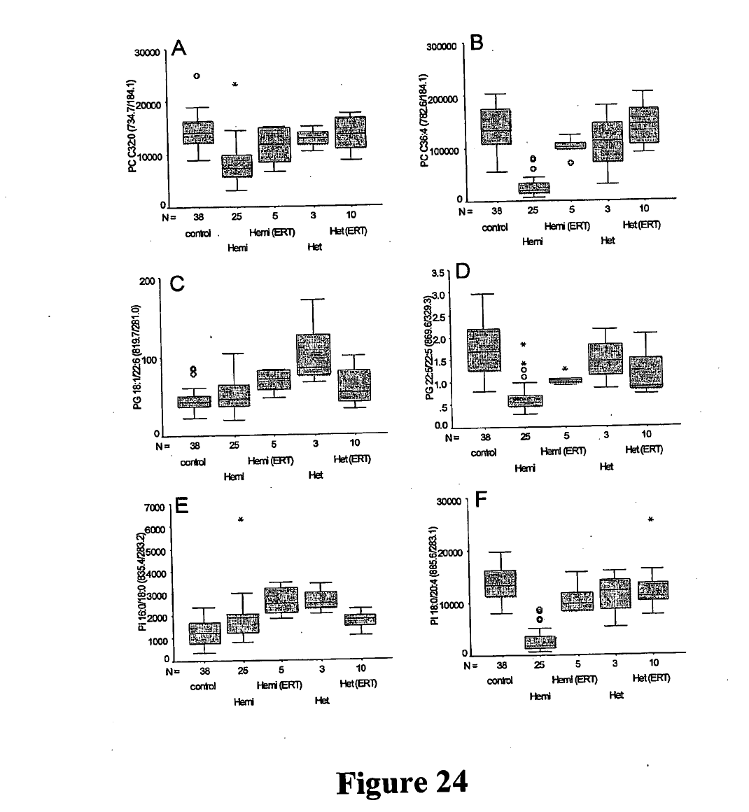

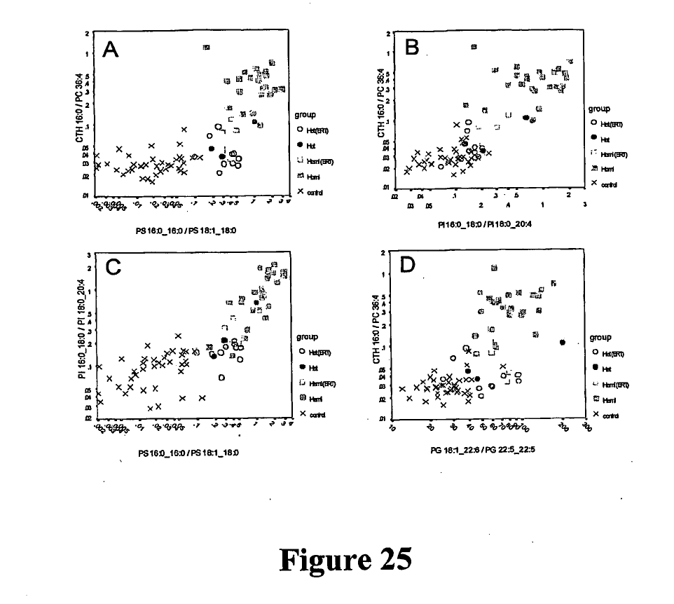

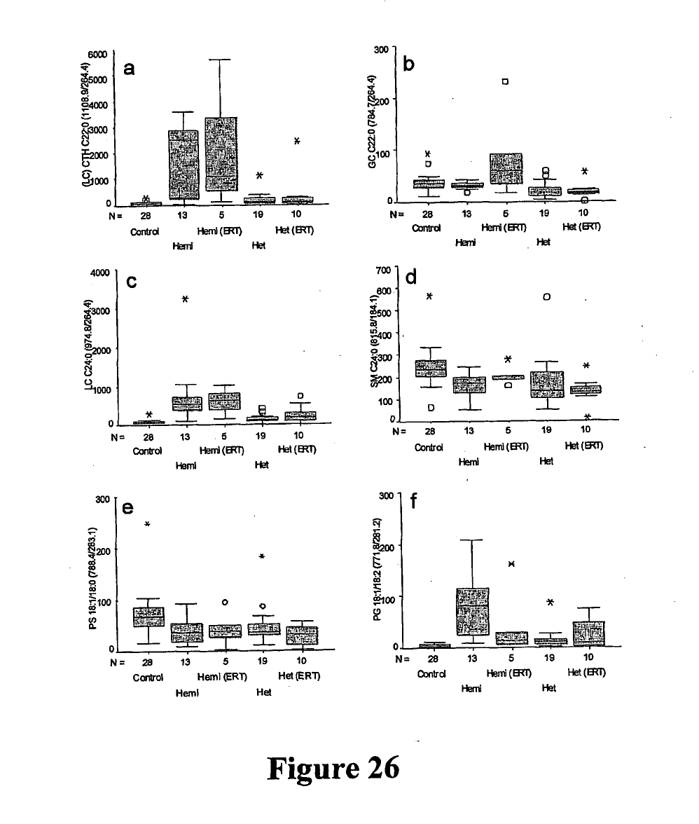

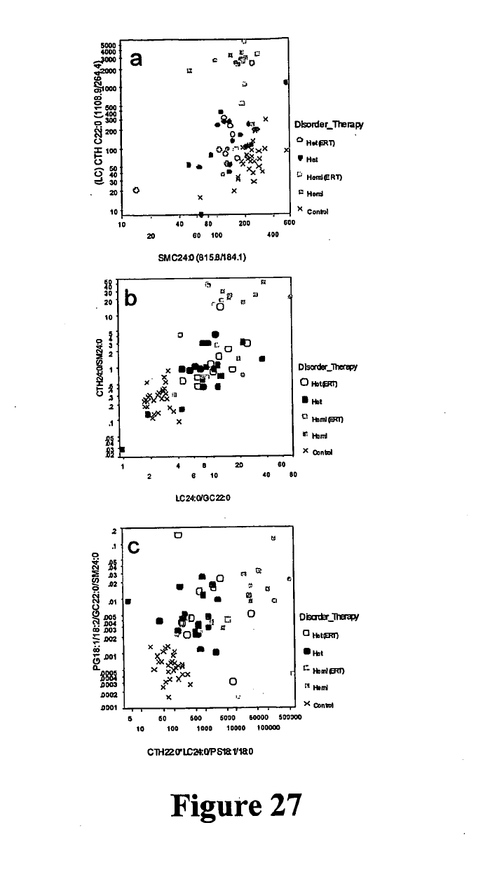

As with the urine data expressed as nmol/L the differentiation between control and patient groups could be improved by the selection of specific lipid species. The increases observed in Cer, LC and CTH were greatest in the C24:1 species, and the decreases observed in GC and SM were greatest in the C24:0 species (FIG. 13). Following these observations we looked at the relationship between these lipid species in a series of scatter plots and how these were able to differentiate the control and patient groups (FIG. 14). Using different combinations we can achieve almost total differentiation between the control and patient groups, particularly with CTH C24:1 and LC C24:1 plotted as a function of SM C24:0 (FIGS. 5D and 5E).

LC and CTH are elevated while GC and SM are decreased in the patient groups. The use of ratios of these analytes enables further discrimination between the control and patient groups. FIG. 15 shows total separation of both Fabry and Fabry het groups from the control group.

Of interest is the observation that the composition of individual PC species is significantly altered in the Fabry group compared to the control group. Some PC species show a proportional elevation (C34:2 and C36:4) while others show a corresponding decrease (C32:1 and C34:1) (FIG. 16). On first examination there appears to be a trend toward higher levels of unsaturated fatty acids in the Fabry group. This is supported by the observation that the LC C24:1 and CTH C24:1 species show a greater elevation in the Fabry group compared to the C24:0 species. The effect of these changes in the lipid composition to the cellular function in Fabry disease and the relationship to the pathophysiology of this disorder is unclear at this time. However, we are further investigating these effects in cultured skin fibroblasts from control and Fabry patients. Results will be available in subsequent Reports.

To summarise, analysis of the lipid profile in urine from control, Fabry and Fabry het groups has identified the specific lipid species, ratios and profiles that best discriminate between the control and patient groups. Correction of the lipid species for PC content of the urine improved the discrimination between control and Fabry groups and minimised the potential for the false identification of individuals with high urinary sediment as Fabry hets. The “Ratio 4” (LC C24:1*CTH C24:1)/(GC C24:0*SM C24:0) provides total discrimination of all Fabry and Fabry hets from the control group.

Analysis of Plasma: The number of plasma and blood spot samples available from the Fabry het group were fewer than the urine samples. However, lipid profiles were performed on these samples and the Mann-Whitney U values for each lipid species are shown in Table 8. No significant difference is observed between the control and Fabry het groups (possibly due to the low number of Fabry het samples), however Cer, LC, CTH and SM species show significant differences between the control and Fabry groups. FIG. 17 shows that Cer, LC and SM are decreased in the Fabry group compared to the control group, while CTH is increased and GC is unchanged, although it did appear to have a broader range in the Fabry group. When the Cer, GC, LC and SM C16:0 species were plotted as a function of the CTH C16:0 (FIG. 18) a strong correlation is observed in the Fabry group, which provides improved discrimination between the control and Fabry groups.

Analysis of Whole Blood: Analysis of dried blood spots for lipids show relatively few analytes with significant differences between the control and Fabry groups (Table 9). Box plots of the lipid groups (FIG. 19) show only slight elevations or decreases in the Fabry compared to the control groups, and only the CTH has a p value of less than 0.05. The use of specific lipid species offers little improvement although the decrease of Cer C24:1 in the Fabry group compared to the control group is significant (p=0.03) (FIG. 20). The box plots of the CTH species show that only the C16:0, C18:0 and C20:0 species are significantly different from the control group (FIG. 21 and Table 9). The scatter plot of CTH C16:0 as a function of Cer C16:0 (FIG. 22A) shows a similar correlation between these two analytes, as was observed in the plasma samples. The correlation is not as pronounced in the plot of CTH C18:0 as a function of SM C16:0 (FIG. 22B). The Fabry het group did not show any significant difference to the control group in the lipid analytes.

### Discussion

The use of a urinary lipid profile also has potential to identify Fabry and Fabry heterozygotes. While the determination of CTH on its own did not identify all patients, the use of ratios of lipid species provided total discrimination of both the Fabry patients (even after renal transplant) and the heterozygotes from the control group. Urine analysis is a practical, non-invasive procedure to screen large populations at high risk for Fabry disease.

Monitoring of therapy: Characterisation of the lipid profile of Fabry patients in plasma, dried blood spots and urine has highlighted a number of previously unreported differences between Fabry patients and the control population. This technology enables us to very accurately describe the lipid profile from the control population and so define how the profile differs in Fabry disease. Significant differences were observed in most lipid groups suggesting that Fabry disease results in a general alteration of lipid metabolism, not just the storage of trihexosylceramide. With further validation it will be possible to monitor therapy in Fabry disease by following the total lipid profile as it is corrected from the disease state to a normal profile. This will provide a more comprehensive Fabry monitoring program than current methods allow. We are currently investigating the potential of this approach with patient samples and cultured skin fibroblasts.

Prediction of disease severity: The detailed description of the disease state provided by the lipid profile described in this Report will significantly improve our ability to describe the disease in any given individual. Correlation of these profiles with known phenotypes and disease progression will enable us to predict disease progression.

TABLE 5
Lipid analytes used for lipid analysis of Fabry samples

| | | MRM ion |
|---|---|---|
| Lipid analytes^{a} | Internal standard | pairs (m/z) |
| Cer C16:0 | Cer C17:0 | 538.7/264.4 |
| Cer C24:0 | Cer C17:0 | 650.7/264.4 |
| Cer C24:1 | Cer C17:0 | 648.7/264.4 |
| Cer C17:0 (internal standard) | | 552.7/264.4 |
| GC C16:0 | GC(d3)C16:0 | 700.6/264.4 |
| GC C22:0 | GC(d3)C16:0 | 784.7/264.4 |
| GC C24:0 | GC(d3)C16:0 | 812.7/264.4 |
| GC C24:1 | GC(d3)C16:0 | 810.8/264.4 |
| GC(d3)C16:0 (internal standard) | | 703.8/264.4 |
| LC C16:0 | LC(d3)C16:0 | 862.4/264.4 |
| LC C20:0 | LC(d3)C16:0 | 918.6/264.4 |
| LC C22:0 | LC(d3)C16:0 | 946.7/264.4 |
| LC C22:0-OH | LC(d3)C16:0 | 962.7/264.4 |
| LC C24:0 | LC(d3)C16:0 | 974.8/264.4 |
| LC C24:1 | LC(d3)C16:0 | 972.8/264.4 |
| LC(d3)C16:0 (internal standard) | | 865.6/264.4 |
| CTH C16:0 | CTH C17:0 | 1024.1/264.4 |
| CTH C18:0 | CTH C17:0 | 1052.1/264.4 |
| CTH C20:0 | cTH C17:0 | 1080.1/264.4 |
| CTH C22:0 | CTH C17:0 | 1108.1/264.4 |
| CTH C24:0 | CTH C17:0 | 1136.6/264.4 |
| CTH C24:1 | CTH C17:0 | 1134.1/264.4 |
| CTH C17:0 (internal standard) | | 1038.1/264.4 |
| SM C16:0 | PC C14:0 | 703.9/184.1 |
| SM C22:O | PC C14:0 | 787.8/184.1 |
| SM C24:0 | PC C14:0 | 815.8/184.1 |
| PC C32:0 | PC C14:0 | 706.5/184.1 |
| PC C32:1 | PC C14:0 | 704.5/184.1 |
| PC C34:1 | PC C14:O | 732.5/184.1 |
| PC C34:2 | PC C14:0 | 730.5/184.1 |
| PC 36:2 | PC C14:0 | 758.6/184.1 |
| PC C36:4 | PCC14:0 | 754.6/184.1 |
| PC C38:4 | PC C14:0 | 782.6/184.1 |
| PC C14:0^{b} (internal standard) | | 678.5/184.1 |

^{a}Cer = ceramide, GC = glucosylceramide, LC = lactosylceramide. CTH = ceramide trihoxoside, SM = sphingomyelin, PC = phosphatidylcholine
^{b}PC C14:0 Is a commercial standard and is known to have a C16:0 second fatty acid (equivalent to PC C30:0)

TABLE 6
Mann-Whitney U values for lipid^{a} analytes in urine.

| | Control (n = 20) vs | | Control (n = 20) vs | |
|---|---|---|---|---|
| | Heterozygote (n = 13) | | Fabry (n = 14) | |
| Analyte^{b} | MW-U | p value | MW-U | p value |
| Cer C16:0 | 41 | 0.000 | 81 | 0.018 |
| Cer C24:0 | 82 | 0.037 | 118 | 0.243 |
| Cer C24:1 | 62 | 0.006 | 68 | 0.005 |
| GC C16:0 | 63 | 0.006 | 144 | 0.746 |
| GC C22:0 | 87 | 0.056 | 119 | 0.256 |
| GC C24:0 | 69 | 0.012 | 118 | 0.243 |
| GC C24:1 | 71 | 0.014 | 153 | 0.974 |
| LC C16:0 | 18 | 0.000 | 61 | 0.003 |
| LC C20:0 | 41 | 0.000 | 56 | 0.001 |
| LC C22:0 | 34 | 0.000 | 77 | 0.012 |
| LC C22:0-OH | 37 | 0.000 | 81 | 0.018 |
| LC C24:0 | 23 | 0.000 | 17 | 0.000 |
| LC C24:1 | 11 | 0.000 | 2 | 0.000 |
| CTH C16:0 | 3 | 0.000 | 56 | 0.001 |
| CTH C18:0 | 61 | 0.005 | 46 | 0.000 |
| CTH C20:0 | 22 | 0.000 | 59 | 0.001 |
| CTH C22:0 | 2 | 0.000 | 43 | 0.000 |
| CTH C24:0 | 4 | 0.000 | 37 | 0.000 |
| CTH C24:1 | 0 | 0.000 | 25 | 0.000 |
| SM C16:0 | 80 | 0.031 | 115 | 0.206 |
| SM C22:0 | 83 | 0.041 | 74 | 0.009 |
| SM C24:0 | 120 | 0.432 | 70 | 0.006 |
| PC C32:0 | 50 | 0.001 | 14.6 | 0.795 |
| PC C32:1 | 56 | 0.003 | 94 | 0.052 |
| PC C34:1 | 65 | 0.008 | 129 | 0.417 |
| PC C34:2 | 63 | 0.006 | 109 | 0.144 |
| PC 36:2 | 61 | 0.005 | 148 | 0.846 |
| PC C36:4 | 64 | 0.007 | 103 | 0.098 |
| PC C38:4 | 74 | 0.018 | 126 | 0.364 |
| Cer (total) | 56 | 0.003 | 109 | 0.083 |
| GC (total) | 64 | 0.007 | 137 | 0.386 |
| LC (total) | 14 | 0.000 | 40 | 0.000 |
| CTH (total) | 0 | 0.000 | 37 | 0.000 |
| SM (total) | 85 | 0.048 | 84 | 0.012 |
| PC (total) | 62 | 0.006 | 164 | 0.975 |

^{a}lipids expressed as nmol/L urine.
^{b}Cer = ceramide, GC = glucosylceramide, LC = lactosylceramide, CTH = trihexosylceramide, SM = sphingomyelin, PC = phosphatidylcholine.

TABLE 7
Mann-Whitney U values for lipid^{a} analytes in urine.
(Corrected for phospatidylcholine content)

| | Control (n = 20) vs | | Control (n = 20) vs | |
|---|---|---|---|---|
| | Heterozygote (n = 13) | | Fabry (n = 14) | |
| Analyte^{b} | MW-U | p value | MW-U | p value |
| Cer C16:0 | 95 | 0.101 | 73 | 0.009 |
| Cer C24:0 | 90 | 0.070 | 107 | 0.127 |
| Cer C24:1 | 141 | 0.946 | 60 | 0.002 |
| GC C16:0 | 133 | 0.733 | 152 | 0.948 |
| GC C22:0 | 62 | 0.006 | 109 | 0.144 |
| GC C24:0 | 63 | 0.006 | 119 | 0.256 |
| GC C24:1 | 89 | 0.065 | 148 | 0.846 |
| LC C16:0 | 37 | 0.000 | 63 | 0.003 |
| LC C2O:0 | 128 | 0.609 | 69 | 0.006 |
| LC C22:0 | 107 | 0.219 | 80 | 0.016 |
| LC C22:0-OH | 125 | 0.539 | 71 | 0.007 |
| LC C24:0 | 62 | 0.006 | 2 | 0.000 |
| LC C24:1 | 34 | 0.000 | 2 | 0.000 |
| CTH C16:0 | 87 | 0.056 | 35 | 0.000 |
| CTH C18:0 | 126 | 0.562 | 33 | 0.000 |
| CTH C2O:0 | 128 | 0.609 | 35 | 0.000 |
| CTH C22:0 | 68 | 0.010 | 26 | 0.000 |
| CTH C24:0 | 78 | 0.026 | 11 | 0.000 |
| CTH C24:1 | 43 | 0.001 | 4 | 0.000 |
| SM C16:0 | 42 | 0.001 | 70 | 0.006 |
| SM C22:0 | 47 | 0.001 | 0 | 0.000 |
| SM C24:0 | 43 | 0.001 | 4 | 0.000 |
| PC C32:0 | 120 | 0.432 | 83 | 0.021 |
| PC C32:1 | 136 | 0.811 | 28 | 0.000 |
| PC C34:1 | 72 | 0.015 | 39 | 0.000 |
| PC C34:2 | 143 | 1.000 | 31 | 0.000 |
| PC 36:2 | 84 | 0.044 | 135 | 0.538 |
| PCC36:4 | 127 | 0.585 | 20 | 0.000 |
| PC C38:4 | 75 | 0.020 | 93 | 0.048 |
| Cer (total) | 119 | 0.413 | 82 | 0.019 |
| GC (total) | 83 | 0.041 | 129 | 0.417 |
| LC (total) | 77 | 0.024 | 26 | 0.000 |
| CTH (total) | 97 | 0.116 | 19 | 0.000 |
| SM (total) | 42 | 0.001 | 12 | 0.000 |

^{a}expressed as nmol/unol PC.
^{b}Cer = ceramide, GC = glucosylceramide, LC = lactosylceramide, CTH = trihexosylceramide, SM = sphingomyelin, PC = phosphatidylcholine.

TABLE 8
Mann-Whitney U values for lipid^{a} analytes in plasma.

| | Control (n = 10) vs | | Control (n = 10) vs | |
|---|---|---|---|---|
| | Heterozygote (n = 2) | | Fabry (n = 29) | |
| Analyte^{b} | MW-U | p value | MW-U | p value |

| | | | | |
|---|---|---|---|---|
| Cer C16:0 | 9 | 0.830 | 59 | 0.007 |
| Cer C24:1 | 7 | 0.519 | 34 | 0.000 |
| Cer C24:0 | 9 | 0.830 | 48 | 0.002 |
| GC C16:0 | 9 | 0.830 | 136.5 | 0.908 |
| GC C22:0 | 9 | 0.830 | 134 | 0.842 |
| GC C24:1 | 6 | 0.390 | 137.5 | 0.934 |
| GC C24:0 | 2 | 0.085 | 124 | 0.596 |
| LC C16:0 | 9 | 0.830 | 66 | 0.014 |
| LC C24:1 | 8 | 0.667 | 33 | 0.000 |
| LC C24:0 | 4 | 0.197 | 4.5 | 0.000 |
| CTH C16:0 | 8 | 0.667 | 33 | 0.000 |
| CTH C18:0 | 7 | 0.519 | 19.5 | 0.000 |
| CTH C20:0 | 9 | 0.830 | 49 | 0.003 |
| CTH C22:0 | 4 | 0.197 | 45 | 0.002 |
| CTH C24:1 | 10 | 1.000 | 49 | 0.003 |
| CTH C24:0 | 8 | 0.667 | 53 | 0.004 |
| SM C16:0 | 10 | 1.000 | 33 | 0.000 |
| SM C22:0 | 4 | 0.197 | 39 | 0.001 |
| SM C24:0 | 8 | 0.667 | 29 | 0.000 |
| Cer (total) | 7 | 0.519 | 38.5 | 0.001 |
| GC (total) | 5 | 0.282 | 138 | 0.947 |
| LC (total) | 8 | 0.667 | 48 | 0.002 |
| CTH (total) | 10 | 1.000 | 38 | 0.001 |
| SM (total) | 8 | 0.667 | 37 | 0.001 |

^{a}lipids were calculated as umol/L plasma.
^{b}Cer = ceramide, GC = glucosylceramide, LC = lactosylceramide, CTH = trihexosylceramide, SM = sphingomyelin.

TABLE 9
Mann-Whitney U values for lipid^{a} analytes in whole blood.

| | Control (n = 10) vs | | Control (n = 10) vs | |
|---|---|---|---|---|
| | Heterozygote (n = 2) | | Fabry (n = 13) | |
| Analyte^{b} | MW-U | p value | MW-U | p value |
| Cer C16:0 | 8 | 0.235 | 48 | 0.292 |
| Cer C24:1 | 8 | 0.237 | 30 | 0.030 |
| Cer C24:0 | 12 | 0.612 | 46.5 | 0.251 |
| GC C16:0 | 14 | 0.866 | 39 | 0.107 |
| GC C22:0 | 7.5 | 0.202 | 40.5 | 0.128 |
| GC C24:1 | 15 | 1.000 | 47.5 | 0.278 |
| GC C24:0 | 9 | 0.310 | 38.5 | 0.100 |
| LC C16:0 | 13 | 0.735 | 37.5 | 0.088 |
| LC C24:1 | 7 | 0.175 | 61.5 | 0.828 |
| LC C24:0 | 12 | 0.612 | 40.5 | 0.129 |
| CTH C16:0 | 10 | 0.398 | 6 | 0.000 |
| CTH C18:0 | 8 | 0.237 | 42.5 | 0.163 |
| CTH C20:0 | 9 | 0.310 | 45 | 0.215 |
| CTH C22:0 | 10 | 0.398 | 40 | 0.121 |
| CTH C24:1 | 7.5 | 0.204 | 1.5 | 0.000 |
| CTH C24:0 | 6 | 0.128 | 32 | 0.041 |
| SM C16:0 | 7.5 | 0.204 | 53.5 | 0.475 |
| SM C22:0 | 9 | 0.310 | 61 | 0.804 |
| SM C24:0 | 11 | 0.499 | 55.5 | 0.556 |
| Cer (total) | 9 | 0.310 | 38 | 0.094 |
| GC (total) | 13 | 0.735 | 37 | 0.082 |
| LC (total) | 11 | 0.499 | 42 | 0.154 |
| CTH (total) | 9 | 0.310 | 23 | 0.009 |
| SM (total) | 12 | 0.612 | 63.5 | 0.926 |

^{a}lipids were calculated as umol/L plasma.
^{b}Cer = ceramide, GC = glucosylceramide, LC = lactosylceramide, CTH = trihexosylceramide, SM = sphingomyelin.

Appendix I: Procedure for Sphingolipid Extraction from Urine (Bligh-Dyer Method).
1. To 1.5 mL urine add 5.6 mL CHCl₃/MeOH (1:2)
2. Add 400 μmol internal standards to each sample; 2 μL (d3) C16:0 LC (200 μM); 2 μL (d3) C16:0 GC (200 μM), and 2 μL GM2 (200 μM), 6.25 μL CTH C17:0 (64 μM); 2 μL Cer C17:0 (200 μM), 2 μL PC (200 μM).
3. Place tubes on platform shaker for 10 minutes at 150 opm. Stand tubes at room temperature for at least 50 minutes.
4. Partition with the addition of 1.9 mL CHCl₃ and 1.9 mL milliQ H₂O or KCl.
5. Place tubes on platform shaker for 10 minutes at 150 opm.
6. Centrifuge at 3000 rpm for 2 minutes then remove and discard upper phase by suction.
7. Wash the lower phase with the addition of 0.5 mL of B&D synthetic upper phase and vortexing briefly.
8. Centrifuge at 3000 rpm for 2 minutes then remove and discard upper phase by suction.
9. Dry samples (lower phase) under N₂ at 40° C. (add water to heating block around tube to aid in evaporation). Periodically vortex the samples during the drying down process to ensure the highest recovery possible.
10. Resuspend extracts in 150 μL of MeOH containing 10 mM ammonium formate.
   Appendix II: Procedure for Glycolipid, Phospholipid and Ganglioside Extraction from Plasma (Folch Extraction).
1. Add 100 μL plasma to a 12 mL glass tube with black screw cap lid.
2. Add 2 mL CHCl₃/MeOH (2:1) (at least 20 volumes of CHCl₃/MeOH to each sample).
3. Add internal standards to each sample 2 μL (d3) C16:0 LC (200 μM); 2 μL (d3) C16:0 GC (200 μM), and 2 μL GM2 (200 μM), 6.25 μL CTH C17:0 (64 μM); 2 μL Cer C17:0 (200 μM; 2 μL PC (200 μM).
4. Shake for 10 minutes at 150 rpm. Stand on the bench at room temperature for at least 50 minutes.
5. Partition with the addition of 0.2 volumes (ie. 0.4 mL) of milliQ H₂O and vortex.
6. Centrifuge at 4000×g for 10 minutes then gently remove upper aqueous layer, transferring it to a clean glass tube with a glass pipette for use in the ganglioside extraction and set aside (refer to ganglioside extraction). Carefully remove and discard the protein interphase.
7. Dry samples (lower phase) under N₂ at 40° C.
8. Resuspend samples in 20 μL methanol and add 0.18 mL CHCl₃ (containing 1% ethanol) and vortex to ensure sample is resuspended.
9. Pre-wash silica reverse phase columns (100 mg) with 3 mL acetone/methanol (9:1) followed by 3 mL CHCl₃ (containing 1% ethanol).
10. Load sample with a glass pipette and allow it to completely enter the solid phase, then wash with 3 mL CHCl₃ (containing 1% ethanol) (neutral lipids (ceramide) will go through and glycolipids/phospholipids will bind to the column).
11. Elute the glycolipids and phospholipids from the column into a clean 12 mL glass tube with black screw cap lid with 3 mL acetone/methanol (9:1) and vacuum dry columns briefly. (LC and GC internal standards are present in this fraction.)
12. Elute the phospholipids from the column into clean 12 mL glass tube with black screw cap lid with 3 mL methanol and vacuum dry columns briefly. (PC internal standard is present in this fraction if used.)
   Note: Omitting step 10 will result in the glycolipids and phospholipids being eluted together.
13. Dry samples under N₂ at 40° C.
14. Resuspend samples in 100 μL MeOH and store at −20° C.
15. Prior to running on the mass spectrometer resuspend samples into a final volume of 200 μL methanol containing 10 mM ammonium formate.

### Ganglioside Extraction

1. Follow glycolipid and phospholipid extraction procedure to step 6, taking upper aqueous phase from Folch extraction following H₂O partition.
2. Prime 25 mg C18 columns with 3×1 mL MeOH, followed by 3×1 mL MQ water.
3. Load upper phase to column with a glass pipette and allow solution to completely enter the solid phase of the column, then wash with 3×1 mL MQ water.
4. Elute gangliosides from the column into a clean 12 mL glass tube with black screw cap lid with 2×1 mL MeOH and vacuum dry columns briefly.
5. Dry samples under N₂ at 40° C.
6. Store samples at −20° C.
7. Prior to running on the mass spectrometer resuspend in 200 μL methanol containing 10 mM ammonium formate.
   Appendix III: Procedure for Extraction of Glycosphingolipids from Guthrie Spots

### Materials and Reagents:

### Isopropanol Standards Mixture:

### 1.0 μM Phosphatidylcholine C14:0/C14:0 (MW=678)

### 1.0 μM Glucosylceramide(d3) C18:0 (MW=703.8)

### 1.0 μM Lactosylceramide(d3) C16:0 (MW=865.6)

### 1.0 μM Ceramide C17:0 (MW=252.7)

1.0 μM Tri-hexose ceramide CTH C17:0 (MW=1038.9)

### 1.0 μM Monosialoganglioside GM2 (MW=1384.9)

1×1 mL 96 deep-well, v-bottom tray (polypropylene) and lid
1×250 μL v-bottom tray
Multichannel pipette

### Plate-shaker

### Experimental Procedure:

1. Place two 3 mm blood spots per sample in each well of a 96 deep-well, v-bottom tray.
2. Add 200 μL isopropanol containing standards (200 μmol of each standard) to each sample.
3. Cover tray with polypropylene plastic lid and shake samples for 2 hours on amplitude setting 09 and form setting 99.
4. Remove 200 μL from samples into a 1×250 μL v-bottom tray leaving blood spots behind.
5. Dry down samples over N₂.
6. Resuspend extracts in 100 μL of MeOH containing 10 mM ammonium formate.
7. Cover plate with alfoil and analyse samples by mass spectrometry.

### Example 3

### Monitoring of Therapy for Gaucher Disease Using Sphingolipid and Phospholipid Analysis

This report provides a detailed analysis of the initial trial of our developed methodology to monitor enzyme replacement therapy (ERT) in Gaucher disease using dried blood spots.

Patient samples: Dried blood spots were collected from Gaucher patients receiving ERT for up to 10 years. In addition, dried blood spots have been collected from patients not receiving ERT. Control samples were collected from healthy individuals. Total sample numbers are as shown in Table 10.

Sample preparation: From each Guthrie card sample 2×3 mm dried blood spots were punched and the lipids were eluted (16 h) with 200 μL of isopropanol containing 200 nmol of each internal standard; Cer C17:0, GC(d3)C16:0, LC(d3)C16:0, PC C14:0, PG C14:0/14:0. The blood spots were removed and the isopropanol dried under a stream of nitrogen. Lipids were redissolved in 100 μL of methanol containing 10 mM NH₄COOH for analysis by mass spectrometry.

Mass spectrometry: Mass spectrometric analysis of lipids was performed using a PE Sciex API 3000 triple-quadrupole mass spectrometer with a turbo-ionspray source and Analyst data system (PE Sciex, Concord, Ontario, Canada). Samples (20 μL) were injected into the electrospray source with a Gilson 233 autosampler using a carrying solvent of methanol at a flow rate of 80 μL/minute. For all analytes nitrogen was used as the collision gas at a pressure 2×10⁻⁵ Torr. Lipids were analysed in +ve ion mode for sphingolipids and phosphatidylcholine and −ve ion mode for all other phospholipids. Determination of lipids was performed using the multiple-reaction monitoring (MRM) mode. Seventeen different glycosphingolipid and ceramide species in addition to 36 phospholipid species were monitored using the ion pairs shown in Table 11 and 12. Each ion pair was monitored for 100 milliseconds and the measurements were repeated and averaged over the injection period. Determination of lipids was achieved by relating the peak height of each lipid ion signal to the peak height of the signal from the corresponding internal standard (Table 11 and 12).

### Results

To determine which analytes were potentially useful markers for monitoring Gaucher disease, the patients were grouped into control (group 1, n=22), Gaucher patients receiving ERT (group 2, n=68), and untreated Gaucher patients (group 3, n=20). Mann-Whitney U values were then calculated for each analyte to determine the difference between the control and untreated patients, control and treated patients, and treated and untreated patients. These results are shown in Table 13.

We observed that, in addition to the expected elevation of glucosylceramide (GC) in the untreated Gaucher patients compared to controls, there were significant differences in the level of ceramide C16:0, CTH C24:0 and the sphingomyelin species C16:0, C22:0 and C24:0 (all significant to the 0.01 level). With the exception of the ceramide C16:0, the same markers also showed a significant difference between treated and untreated Gaucher patients. Of the lactosylceramide species only the C16:0 and C22:0-OH species showed a significant difference between control and untreated patients (significant to the 0.05 level) (Table 13). While the GC and ceramide species were elevated in the Gaucher patient group compared to the control group, the LC, CTH and SM species showed a decrease in the Gaucher group. Many of the phospholipid species showed a significant difference between the control and Gaucher groups All of the phosphatidylcholine, phosphatidylserine and phosphatidylethanolamine species and many of the phosphatidylglycerol and phosphatidylinositol species were significantly decreased in the Gaucher patient group compared to the control group (Table 13). Many of these analytes were also decreased in the treated Gaucher patient group. For those analytes where a significant difference was observed between the control and Gaucher groups, the levels in the treated patients generally fell between the control and untreated patients. In each case ERT has partially normalised the lipid levels, although not in all patients.

Although the observed differences between control and untreated patients are significant there is still considerable overlap between the two populations. This is due, at least in part, to the range of lipid levels in the control and patient groups. To improve the discrimination of the markers we investigated the use of multiple markers by calculating Mann-Whitney U values for a number of ratios of different lipid species (Table 14).

In all ratios the Mann-Whitney U values were decreased compared to the GC C16:0 values or other single analytes (compare Table 14 with Table 13). Clearly, the use of multiple analytes or lipid profiles provides a better representation of lipid metabolism in control and Gaucher patients.

Discussion: In this study we have provided evidence that the primary storage substrate GC is a useful marker for monitoring Gaucher disease. We observe an increased level of GC in dried blood spots from untreated patients compared to controls and a normalisation of GC levels after ERT. This is an expected outcome, based on the known biochemistry of Gaucher disease. Somewhat less expected is the elevation in ceramide and the decrease in LC and sphingomyelin. We have previously reported that LC is decreased in the plasma of Gaucher patients and that the ratio of GC/LC provides a better discrimination of Gaucher patients from controls than the GC levels on their own (Whitfield et al 2002). In these preliminary studies we have identified that other lipids are also affected, these include not only ceramide and sphingomyelin but also a number of phospholipids. We have also shown that using a combination of these analytes with the GC and LC levels, provides greater discrimination and potentially a better mechanism for monitoring ERT in Gaucher disease than the use of individual analytes.

TABLE 10
Patient and control samples included in this trial

| Patient group | Number |
|---|---|
| Control | 19 |
| Treated Gaucher | 68 |
| Untreated Gaucher | 20 |

TABLE 11
Lipid analytes used for Gaucher Monitoring

| | | MRM ion |
|---|---|---|
| Lipid analytes^{a} | Internal standard | pairs (m/z) |
| Cer C16:0 | Cer 017:0 | 538.7/264.4 |
| Cer C24:C) | Cer 017:0 | 650.7/264.4 |
| Cer C24:1 | Cer 017:0 | 648.7/264.4 |
| Cer C17:0 (internal standard) | | 552.7/264.4 |
| GC C16:0 | GC(d3)C16:0 | 700.6/264.4 |
| GC C22:0 | GC(d3)C16:0 | 784.7/264.4 |
| GC C24:0 | GC(d3)C16:0 | 812.7/264.4 |
| GC C24:1 | GC(d3)C16:0 | 810.8/264.4 |
| GC(d3)C16:0 (internal standard) | | 703.8/264.4 |
| LC C16:0 | LC(d3)C16:0 | 862.4/264.4 |
| LC C24:0 | LC(d3)C16:0 | 974.8/264.4 |
| LO C24:1 | LC(d3)C16:0 | 972.8/264.4 |
| CTH C16:0 | LC(d3)C16:0 | 1024.1/264.4 |
| CTH C22:0 | LC(d3)C16:0 | 1108.1/264.4 |
| CTH C24:0 | LC(d3)C16:0 | 1136.6/264.4 |
| CTH C24:1 | LC(d3)C16:0 | 1134.1/264.4 |
| LC(d3)016:0 (internal standard) | | 865.6/264.4 |
| SM C16:0 | PC C14:0 | 703.9/184.1 |
| SM C22:0 | PC C14:0 | 787.8/184.1 |
| SM C24:0 | PC C14:0 | 815.8/184.1 |
| PC C14:0 (internal standard) | | 678.5/184.1 |

^{a}Cer = ceramide, GC = glucosylceramide, LC lactosylceramide, CTH = ceramide trihoxoside, SM = sphingomyelin, PC = phosphatidylcholine

TABLE 12
Phospholipid analytes used for Gaucher Monitoring

| | | MRM ion |
|---|---|---|
| Lipid analytes^{a} | Internal standard | pairs (m/z) |
| PC C32:0 | PC C14:0 | 734.7/184 |
| PC C32:1 | PC C14:0 | 732.7/184 |
| PC C34:1 | PC C14:0 | 760.6/184 |
| PC C34:2 | PC C14:0 | 758.5/184 |
| PC C36:2 | PC C14:0 | 786.6/184 |
| PC C36:4 | PC C14:0 | 782.6/184 |
| PC C38:4 | PC C14:0 | 810.8/184 |
| PC C14:0 (internal standard) | | 678.5/184 |
| PE C18:0/20:4 | PG C14:0/14:0 | 766.6/303.4 |
| PE C18:1/18:1 | PG C14:0/14:0 | 742.6/281.1 |
| PG C16:O/18:1 | PG C14:O/14:0 | 747.6/255.8 |
| PG C16:0/22:6 | PG C14:0/14:0 | 793.5/255.5 |
| PG C16:1/18:1 | PG C14:0114:0 | 745.5/281.5 |
| PG C16:1/20:4 | PG C14:0/14:0 | 767.4/253.5 |
| PG C18:1/18:0 | PG C14:0114:0 | 775.6/281.0 |
| PG C18:1/18:1 | PG C14:0/14:0 | 773.4/281.0 |
| PG C18:1/18:2 | PG C14:0/14:0 | 771.8/281.2 |
| PG C18:1/20:4 | PG C14:0/14:0 | 795.6/303.5 |
| PG C18:1/22:5 | PG C14:O/14:0 | 821.8/281.0 |
| PG C18:1/22:6 | PG C14:0/14:O | 819.7/281.0 |
| PG C18:2/22:6 | PG C14:0/14:0 | 817.6/279.0 |
| PG C20:4/22:6 | PG C14:0/14:0 | 841.5/303.5 |
| PG C22:6/22:5 | PG C14:0/14:0 | 867.5/329.3 |
| PG C22:6/22:6 | PG C14:0/14:0 | 865.6/327.1 |
| PI C16:0/18:0 | PG C14:0/14:0 | 835.4/283.2 |
| PI C16:0/20:4 | PG C14:0/14:0 | 857.6/255.2 |
| PI C18:0/18:0 | PG C14:0/14:0 | 865.6/283.3 |
| PI C18:0/18:1 | PG C14:0/14:0 | 863.6/283.1 |
| PI C18:0/20:4 | PG C14:0/14:0 | 885.6/283.1 |
| PI C18:0/22:4 | PG C14:0/14:0 | 913.7/283.6 |
| PI C18:0/22:5 | PG C14;0/14:O | 911.6/283.3 |
| PI C18:1/18:1 | PG C14:0/14:0 | 861.4/281.1 |
| PI C18:1/20:4 | PG C14:0/14:0 | 883.6/281.2 |
| PS C16:0/16:0 | PG C14:0/14:0 | 734.3/255.5 |
| PS C18:0/20:4 | PG C14:0/14:0 | 810.6/283.3 |
| PS C18:1/18:0 | PG C14:0/14:O | 788.4/283.1 |
| PG C14:0/14:0 (internal standard) | | 591.5/227.4 |

^{a} PC = phosphatidylcholine, PG = phosphatidylglycerol, PI = phosphatidylinositol, PS = phosphatidylserine, PE phosphatidylethanolamine

TABLE 13
Mann-Whitney U values for lipid analytes between controls^{a},
untreated Gaucher patients^{b} and Gaucher patients treated with
enzyme replacement therapy^{c}.

| | | | Control vs | | Gaucher vs | |
|---|---|---|---|---|---|---|
| | Control vs | | Gaucher | | Gaucher | |
| | Gaucher | | treated | | Treated | |
| Analyte^{d} | M-W U^{e} | Sig.^{f} | M-W U | Sig. | M-W U | Sig. |
| Cer C16:0 | 37 | 0.000 | 294 | 0.000 | 584 | 0.339 |
| Cer C24:0 | 114 | 0.033 | 481 | 0.090 | 597 | 0.409 |
| Cer C24:1 | 153 | 0.299 | 589 | 0.558 | 627 | 0.598 |
| GC C16:0 | 25 | 0.000 | 310 | 0.001 | 332 | 0.001 |
| GC C22:0 | 112 | 0.028 | 580 | 0.498 | 488 | 0.056 |
| GC C24:0 | 125 | 0.068 | 606 | 0.681 | 493 | 0.063 |
| GC C24:1 | 71 | 0.001 | 334 | 0.001 | 391 | 0.004 |
| LC C16:0 | 120 | 0.049 | 556 | 0.355 | 534 | 0.146 |
| LC C20;0 | 163 | 0.448 | 595 | 0.600 | 544 | 0.176 |
| LC C22:0 | 178 | 0.736 | 589 | 0.558 | 667 | 0.897 |
| LC C22:0-OH | 111 | 0.026 | 578 | 0.485 | 494 | 0.064 |
| LC C24:0 | 187 | 0.933 | 625 | 0.829 | 665 | 0.881 |
| LC C24:1 | 166 | 0.500 | 596 | 0.607 | 670 | 0.921 |
| (LC) CTH C16:0 | 124 | 0.064 | 594 | 0.593 | 508 | 0.087 |
| (LC) CTH C18:0 | 174 | 0.653 | 563 | 0.394 | 622 | 0.564 |
| (LC) CTH C20:0 | 139 | 0.152 | 440 | 0.034 | 611 | 0.492 |
| (LC) CTH C22:0 | 115 | 0.035 | 573 | 0.453 | 486 | 0.053 |
| (LC) CTH C24:0 | 76 | 0.001 | 462 | 0.059 | 418 | 0.009 |
| (LC) CTH C24:1 | 131 | 0.097 | 581 | 0.504 | 390 | 0.004 |
| (1134.9/264.4) | | | | | | |
| SM C16:0 | 69 | 0.001 | 497 | 0.126 | 379 | 0.003 |
| SM C22:0 | 68 | 0.001 | 479 | 0.086 | 397 | 0.005 |
| SM C24:0 | 85 | 0.003 | 353 | 0.003 | 464 | 0.031 |
| PC C32:0 | 161 | 0.415 | 521 | 0.199 | 475 | 0.041 |
| PC C32:1 | 47 | 0.000 | 236 | 0.000 | 678 | 0.984 |
| PC C34:1 | 82 | 0.002 | 338 | 0.002 | 553 | 0.206 |
| PC C34:2 | 70 | 0.001 | 432 | 0.028 | 437 | 0.016 |
| PC C36:2 | 69 | 0.001 | 503 | 0.142 | 384 | 0.003 |
| PC C36:4 | 48 | 0.000 | 322 | 0.001 | 401 | 0.005 |
| PC C38:4 | 56 | 0.000 | 431 | 0.027 | 362 | 0.002 |
| PE 18:0/20:4 | 54 | 0.000 | 509 | 0.025 | 325 | 0.000 |
| (766.6/303.4) | | | | | | |
| PE 18:1/18:1 | 97 | 0.002 | 430 | 0.003 | 475.5 | 0.042 |
| (742.6/281.1) | | | | | | |
| PG 16:0/18:1 | 160 | 0.131 | 715 | 0.757 | 538 | 0.157 |
| (747.6/255.8) | | | | | | |
| PG 16:0/22:6 | 136.5 | 0.035 | 701 | 0.659 | 480 | 0.046 |
| (793.5/255.5) | | | | | | |
| PG 16:1/18:1 | 97 | 0.002 | 386 | 0.001 | 541 | 0.166 |
| (745.5/281.5) | | | | | | |
| PG 16:1/20:4 | 127 | 0.019 | 319 | 0.000 | 562 | 0.240 |
| (767.4(253.5) | | | | | | |
| PG 18:1/18:0 | 133 | 0.028 | 604 | 0.176 | 539 | 0.160 |
| (775.6/281.0) | | | | | | |
| PG 18:1/18:1 | 199 | 0.597 | 649 | 0.353 | 527 | 0.128 |
| (773.4/281.0) | | | | | | |
| PG 18:1/18:2 | 104 | 0.003 | 488 | 0.015 | 520 | 0.111 |
| (771.8/281.2) | | | | | | |
| PG 18:1/20:4 | 104 | 0.003 | 739 | 0.933 | 349 | 0.001 |
| (795.6/303.5) | | | | | | |
| PG 18:1/22.:5 | 146 | 0.062 | 598 | 0.159 | 578 | 0.310 |
| (821.8/281.0) | | | | | | |
| PG 18:1/22:6 | 140 | 0.044 | 540 | 0.051 | 600 | 0.426 |
| (819.7/281.0) | | | | | | |
| PG 18:2/22:6 | 99 | 0.002 | 601 | 0.168 | 419 | 0.009 |
| (817.6/279.0) | | | | | | |
| PG 20:4/22:6 | 82 | 0.001 | 692 | 0.599 | 316 | 0.000 |
| (841.5/303.5) | | | | | | |
| PG 22:6/22:5 | 168 | 0.190 | 669.5 | 0.461 | 555 | 0.213 |
| (867.5/329.3) | | | | | | |
| PG 22:6/22:6 | 174 | 0.247 | 491 | 0.016 | 605 | 0.455 |
| (865.6/327.1) | | | | | | |
| PI 16:0/18:0 | 107 | 0.004 | 515 | 0.029 | 483 | 0.050 |
| (835.4/283.2) | | | | | | |
| PI 16:0/20:4 | 96 | 0.002 | 532 | 0.043 | 501 | 0.075 |
| (857.6/255.2) | | | | | | |
| PI 18:0/18:0 | 125 | 0.017 | 463 | 0.007 | 617 | 0.530 |
| (865.6/283.3) | | | | | | |
| PI 18:0/18:1 | 69 | 0.000 | 359 | 0.000 | 607 | 0.467 |
| (863.6/283.1) | | | | | | |
| PI 18:0/20:4 | 114 | 0.008 | 438 | 0.004 | 559 | 0.228 |
| (885.6/283.1) | | | | | | |
| PI 18:0/22:4 | 166 | 0.174 | 488 | 0.015 | 671 | 0.929 |
| (913.7/283.6) | | | | | | |
| PI 18:0/22:5 | 78 | 0.000 | 215 | 0.000 | 620 | 0.550 |
| (911.6/283.3) | | | | | | |
| PI 18:1/18:1 | 99 | 0.002 | 499 | 0.019 | 522 | 0.116 |
| (861.4/281.1) | | | | | | |
| PI 18:1/20:4 | 132 | 0.027 | 557 | 0.073 | 566 | 0.256 |
| (883.6/281.2) | | | | | | |
| PS 16:0/16:0 | 188 | 0.420 | 589 | 0.135 | 605 | 0.455 |
| (734.3/255.5) | | | | | | |
| PS 18:0/20:4 | 85 | 0.001 | 417 | 0.002 | 444 | 0.019 |
| (810.6/283.3) | | | | | | |
| PS 18:1/18:0 | 81 | 0.000 | 409 | 0.001 | 556 | 0.217 |
| (788.4/283.1) | | | | | | |
| Total Cer | 150 | 0.261 | 597 | 0.615 | 632 | 0.633 |
| Total GC | 49 | 0.000 | 330 | 0.001 | 362 | 0.002 |
| Total LC | 170 | 0.574 | 619 | 0.781 | 630 | 0.619 |
| Total CTH | 103 | 0.015 | 519 | 0.192 | 475 | 0.041 |
| Total SM | 68 | 0.001 | 443 | 0.037 | 399 | 0.005 |
| Total PC | 75 | 0.001 | 397 | 0.011 | 445 | 0.019 |

^{a}controls n = 22
^{b}untreated n = 20
^{c}treated n = 68
^{d}Cer = ceramide, GC = glucosylceramide, LC = lactosylceramide, CTH = ceramide trihoxoside, SM = sphingomyelin, PC = phosphatidylcholine, PG = phosphatidylglycerol, PI = phosphatidylinositol, PS = phosphatidylserine, PE phosphatidylethanolamine
^{c}Mann-Whitney U values
^{f}significance (two-tailed)

TABLE 14
Mann-Whitney U values for lipid analyte ratios between controls^{a}, untreated
Gaucher patients^{b} and Gaucher patients treated with enzyme replacement therapy^{c}.

| | Control vs | | Control vs | | Treated vs | |
|---|---|---|---|---|---|---|
| | Gaucher | | Treated | | untreated | |
| Analyte Ratio | M-W U | Sig. | M-W U | Sig. | M-W U | Sig. |
| GC C16:0/PE 18:0/20:4 | 28 | 0.000 | 241 | 0.000 | 291 | 0.000 |
| GC C16:0/PG 18:1/18:2 | 25 | 0.000 | 260 | 0.000 | 322 | 0.000 |
| GC C16:0/PG 20:4/20:6 | 19 | 0.000 | 344 | 0.002 | 229 | 0.000 |
| GC C16:0/PI 18:0/18:1 | 20 | 0.000 | 184 | 0.000 | 373 | 0.002 |
| (Cer C16:0*GC C16:0)/ | 17 | 0.000 | 157 | 0.000 | 259 | 0.000 |
| (CTH C24:0*SM C16:0) | | | | | | |
| (Cer C16:0*GC C16:0)/ | 23 | 0.000 | 205 | 0.000 | 307 | 0.000 |
| (CTH C24:0*SM | | | | | | |
| C16:0*PC32: 1*PG20:4/22:6* | | | | | | |
| PI18:0/18:1) | | | | | | |
| (Cer C16:0*GC C16:0)/(PC | 12 | 0.000 | 159 | 0.000 | 366 | 0.002 |
| 32:1*PG 20:4/22:6*PI | | | | | | |
| 18:0/18:1) | | | | | | |

^{a}controls n = 22
^{b}untreated n = 20
^{c}treated n = 68
^{d}Cer = ceramide, GC = glucosylceramide, LC = lactosylceramide, CTH = ceramide trihoxoside, SM = sphingomyelin, PC = phosphatidylcholine, PG = phosphatidylglycerol, PI = phosphatidylinositol, PS = phosphatidylserine, PE phosphatidylethanolamine
^{c}Mann-Whitney U values
^{f}significance (two-tailed)

### Example 4

### Diagnosis of Fabry Disease Using Sphingolipid and Phospholipid Analysis

This report summarises the results of analyses performed on urine, from controls, Fabry and Fabry heterozygotes, including analysis of phospholipids.

### Materials and Methods

Patient samples: Urine samples have been collected from 14 Fabry patients (two of whom have had renal transplants), 14 Fabry heterozygotes (three of whom had reported clinical symptoms) and 29 unaffected controls.

Sample preparation and analysis: Urine samples were prepared as described

To 1.5 mL urine add 5.6 mL CHCl₃/MeOH (1:2)

Add 400 μmol internal standards to each sample; 2 μL (d3) C16:0 LC (200 μM); 2 μL (d3) C16:0 GC (200 μM), 2 μL Cer C17:0 (200 μM), 2 μL PC (200 μM), 2 μL PG (200 μM) and 2 μL PI (200 μM).

Place tubes on platform shaker for 10 minutes at 150 opm. Stand tubes at room temperature for at least 50 minutes.

Partition with the addition of 1.9 mL CHCl₃ and 1.9 mL milliQ H₂O or KCl.

Place tubes on platform shaker for 10 minutes at 150 opm.

Centrifuge at 3000 rpm for 2 minutes then remove and discard upper phase by suction.

Wash the lower phase with the addition of 0.5 mL of Bligh-Dyer synthetic upper phase and vortexing briefly.

Centrifuge at 3000 rpm for 2 minutes then remove and discard upper phase by suction. Dry samples (lower phase) under N₂ at 40° C. (add water to heating block around tube to aid in evaporation). Periodically vortex the samples during the drying down process to ensure the highest recovery possible.

Resuspend extracts in 150 μL of MeOH containing 10 mM ammonium formate.

Mass spectrometry: Mass spectrometric analysis of lipids was performed using a PE Sciex API 3000 triple-quadrupole mass spectrometer with a turbo-ionspray source and Analyst data system (PE Sciex, Concord, Ontario, Canada). Samples (20 μL) were injected into the electrospray source with a Gilson 233 autosampler using a carrying solvent of methanol at a flow rate of 80 μL/minute. For all analytes nitrogen was used as the collision gas at a pressure 2×10′ Torr. Lipids were analysed in +ve ion mode for sphingolipids and phosphatidylcholine and −ve ion mode for all other phospholipids. Determination of lipids was performed using the multiple-reaction monitoring (MRM) mode. Seventeen different glycosphingolipid and ceramide species in addition to 36 phospholipid species were monitored using the ion pairs shown in Table 15 and 16. Each ion pair was monitored for 100 milliseconds and the measurements were repeated and averaged over the injection period. Determination of lipids was achieved by relating the peak height of each lipid ion signal to the peak height of the signal from the corresponding internal standard

### Results

Analysis of Urine: Lipid profiling of the urine samples from control, Fabry and Fabry heterozygotes (Fabry het) has been performed. In all, 52 lipid species were determined including ceramide (Cer), glucosylceramide (GC), lactosylceramide (LC), trihexosylceramide (CTH), sphingomyelin (SM) and phosphatidylcholine (PC), phosphatidylglygerol (PG), phosphatidylinositol (PI), phosphatidylethanolamine (PE) and phosphatidylserine (PS) species. Appropriate internal standards were used that provide quantification of these species (expressed as nmol/L urine). PC was included as a general marker of urinary sediment and all lipid species were subsequently corrected for total PC content and expressed as nmol/umol PC.

Table 17 shows the Mann-Whitney U values for each of the two patient groups compared to the control group and of the patient groups compared to each other. The data shows multiple analytes to be significantly different between the control and patient groups. Primarily LC CTH, PC and PG species show major differences between control and Fabry groups. Fewer species show significant differences between control and Fabry Het groups but still 11 lipid species show a significance less than 0.01.

Table 18 shows the Mann-Whitney U values for different lipid ratios involving 2 or more lipid species. In most instances the ratios provide better discrimination than the individual analytes involved (based on the Mann-Whitney U values.

### Discussion

In this study we have provided evidence that the primary storage substrate CTH is a useful marker for diagnosis of Fabry disease. We observe an increased level of CTH in urine from most Fabry patients. This is an expected outcome, based on the known biochemistry of Fabry disease. Somewhat less expected is the elevation in all of the PC and PG species as well as two ceramide species and two of the three sphingomyelin species. In these preliminary studies we have identified that in addition to CTH, other lipids are also affected, these include not only ceramide and sphingomyelin but also a number of phospholipids. We have also shown that using a combination of these analytes either alone or with the CTH levels, provides greater discrimination and potentially a better mechanism for diagnosis of Fabry and identification of Fabry heterozygotes than the use of individual analytes.

TABLE 15
Lipid analytes used for Fabry urine analysis

| | | MRM ion |
|---|---|---|
| Lipid analytes^{a} | Internal standard | pairs (m/z) |
| Cer C16:O | Cer C17:0 | 538.7/264.4 |
| Cer C24:0 | Cer C17:0 | 650.7/264.4 |
| Cer C24:1 | Cer C17:0 | 648.7/264.4 |
| Cer C17:0 (internal standard) | | 552.7/264.4 |
| GC C16:0 | GC(d3)C16:0 | 700.6/264.4 |
| GC C22:0 | GC(d3)C16:0 | 784.7/264.4 |
| GC C24:0 | GC(d3)C16:0 | 812.7/264.4 |
| GC C24:1 | GC(d3)C16:0 | 810.8/264.4 |
| GC(d3)C16:0 (internal standard) | | 703.8/264.4 |
| LC C16:0 | LC(d3)C16:0 | 862.4/264.4 |
| LC C24:0 | LC(d3)C16:0 | 974.8/264.4 |
| LC C24:1 | LC(d3)C16:0 | 972.8/264.4 |
| CTH C16:0 | LC(d3)C16:0 | 1024.1/264.4 |
| CTH C22:0 | LC(d3)C16:0 | 1108.1/264.4 |
| CTH C24:0 | LC(d3)C16:0 | 1136.6/264.4 |
| CTH C24:1 | LC(d3)C16:0 | 1134.1/264.4 |
| LC(d3)C16:0 (internal standard) | | 865.6/264.4 |
| SM C16:0 | PC C14:0 | 703.9/184.1 |
| SM C22:0 | PC C14:0 | 787.8/184.1 |
| SM C24:0 | PC C14:0 | 815.8/184.1 |
| PC C14:0 (internal standard) | | 678.5/184.1 |

^{a}Cer = ceramide, GC = glucosylceramide, LC = lactosylceramide, CTH = ceramide trihoxoside, SM = sphingomyelin, PC = phosphatidylcholine

TABLE 16
Phospholipid analytes used for Fabry urine analysis.

| | | MRM ion |
|---|---|---|
| Lipid analytes^{a} | Internal standard | pairs (m/z) |
| PC C32:0 | PC C14:0 | 734.7/184 |
| PC C32:1 | Pc C14:0 | 732.7/184 |
| PC C34:1 | PC C14:0 | 760.6/184 |
| PC C34:2 | PC C14:0 | 758.5/184 |
| PC C36:2 | PC C14:0 | 786.6/184 |
| PC C36:4 | PC C14:0 | 782.6/184 |
| PC C38:4 | PC C14:0 | 810.8/184 |
| PC C14:0 (internal standard) | | 678.5/184.1 |
| PE C18:0/20:4 | PG C14:0/14:0 | 766.6/303.4 |
| PE C18:1/18:1 | PG C14:0/14:0 | 742.6/281.1 |
| PG C16:0/18:1 | PG C14:0/14:0 | 747.6/255.8 |
| PG C16:0/22:6 | PG C14:O/14:0 | 793.5/255.5 |
| PG C16:1/18:1 | PG C14:0/14:0 | 745.5/281.5 |
| PG C16:1/20:4 | PG C14:0/14:0 | 767.4/253.5 |
| PG C18:1/18:0 | PG C14:0/14:0 | 775.6/281.0 |
| PG C18:1/18:1 | PG C14:0/14:0 | 773.4/281.0 |
| PG C18:1/18:2 | PG C14:0/14:0 | 771.8/281.2 |
| PG C18:1/20:4 | PG C14:0/14:0 | 795.6/303.5 |
| PG C18:1/22:5 | PG C14:0/14:0 | 821.8/281.0 |
| PG C18:1/22:6 | PG C14:0/14:0 | 819.7/281.0 |
| PG C18:2/22:6 | PG C14:0/14:0 | 817.6/279.0 |
| PG C20:4/22:6 | PG C14:0/14:0 | 841.5/303.5 |
| PG C22:6/22:5 | PG C14:0/14:0 | 867.5/329.3 |
| PG C22:6/22:6 | PG C14:0/14:0 | 865.6/327.1 |
| PG C14:0/14:0 (internal standard) | | 591.5/227.4 |
| PI C16:0/18:0 | PI C16:0/16:0 | 835.4/283.2 |
| PI C16:0/20:4 | PI C16:0/16:0 | 857.6/255.2 |
| PI C18:0/18:0 | PI C16:0/16:0 | 865.6/283.3 |
| PI C18:0/18:1 | PI C16:0/16:0 | 863.6/283.1 |
| PI C18:0/20:4 | PI C16:0/16:0 | 885.6/283.1 |
| PI C18:0/22:4 | PI C16:0/16:0 | 913.7/283.6 |
| PI C18:0/22:5 | PI C16:0/16:0 | 911.6/283.3 |
| PI C18:1/18:1 | PI C16:0/16:0 | 861.4/281.1 |
| PI C18:1/20:4 | PI C16:0/16:0 | 883.6/281.2 |
| PI C14:0/14:0 (internal standard) | | 751.5/227.4 |
| PS C16:0/16:0 | PG C14:0/14:0 | 734.3/255.5 |
| PS C18:0/20:4 | PG C14:0/14:0 | 810.6/283.3 |
| PS C18:1/18:0 | PG C14:0/14:0 | 788.41283.1 |

^{a}PC = phosphatidylcholine, PG = phosphatidylglycerol, PI = phosphatidylinositol, PS = phosphalidylserine, PE phosphatidylethanolamine

TABLE 17
Mann-Whitney U values for lipid analytes between contols^{a}, Fabry^{b} and
Fabry Hets^{c}.

| | Cont vs | | Cont vs | | Fabry vs | |
|---|---|---|---|---|---|---|
| | Fabry | | Het | | Het | |
| Analyte^{d} | M-W U^{e} | Sig^{f} | M-W U | Sig | M-W U | Sig |
| Cer C16:0 (538.7/264.4) | 119 | 0.029 | 189 | 0.717 | 62 | 0.098 |
| Cer C24:0 (650.7/264.4) | 132 | 0.066 | 175 | 0.468 | 52 | 0.035 |
| Cer C24:1 (648.7/264.4) | 70 | 0.001 | 187 | 0.678 | 37 | 0.005 |
| Cer C20:0 (592.7/264.4) | 155 | 0.213 | 168 | 0.364 | 59 | 0.073 |
| Cer C20:1 (590.7/264.4) | 193 | 0.795 | 124 | 0.041 | 46 | 0.017 |
| Cer C23:0 (636.7/264.4) | 144 | 0.126 | 143 | 0.120 | 53 | 0.039 |
| Cer C23:1 (634.8/264.4) | 160 | 0.265 | 146 | 0.140 | 51 | 0.031 |
| GC C16:0 (700.6/264.4) | 203 | 1.000 | 148 | 0.154 | 70 | 0.198 |
| GC C22:0 (784.7/264.4) | 152 | 0.186 | 89 | 0.003 | 48 | 0.022 |
| GC C24:0 (812.7/264.4) | 182 | 0.586 | 101 | 0.008 | 60 | 0.081 |
| GC C24:1 (810.8/264.4) | 137 | 0.087 | 143 | 0.120 | 41 | 0.009 |
| LC C16:0 (862.4/264.4) | 107 | 0.013 | 117 | 0.026 | 93 | 0.818 |
| LC C20:0 (918.7/264.4) | 66 | 0.000 | 196 | 0.856 | 29 | 0.002 |
| LC C22:0 (946.7/264.4) | 70 | 0.001 | 151 | 0.178 | 53 | 0.039 |
| LC C22:0-OH (962.7/264.4) | 75 | 0.001 | 166 | 0.338 | 44 | 0.013 |
| LC C24:0 (974.8/264.4) | 11 | 0.000 | 100 | 0.008 | 19 | 0.000 |
| LC C24:1 (972.8/264.4) | 41 | 0.000 | 98 | 0.007 | 66 | 0.141 |
| (LC) CTH C16:0 (1024.8/264.4) | 41 | 0.000 | 143 | 0.120 | 39 | 0.007 |
| (LC) CTH C18:0 (1052.7/264.4) | 18 | 0.000 | 157 | 0.233 | 20 | 0.000 |
| (LC) CTH C20:0 (1080.9/264.4) | 75 | 0.001 | 197 | 0.876 | 32 | 0.002 |
| (LC) CTH C22:0 (1108.9/264.4) | 47 | 0.000 | 96 | 0.006 | 48 | 0.022 |
| (LC) CTH C24:0 (1136.9/264.4) | 26 | 0.000 | 111 | 0.017 | 34 | 0.003 |
| (LC) CTH C24:1 (1134.9/264.4) | 43 | 0.000 | 106 | 0.012 | 46 | 0.017 |
| PC C32:0 (734.7/184.1) | 118 | 0.028 | 166 | 0.338 | 77 | 0.335 |
| PC C32:1 (732.7/184.1) | 58 | 0.000 | 167 | 0.351 | 55 | 0.048 |
| PC C34:1 (760.6/184.1) | 83 | 0.002 | 113 | 0.020 | 87 | 0.613 |
| PC C34:2 (758.5/184.1) | 86 | 0.002 | 183 | 0.604 | 34 | 0.003 |
| PC C36:2 (786.6/184.1) | 125 | 0.043 | 130 | 0.058 | 82 | 0.462 |
| PC C36:4 (782.6/184.1) | 87 | 0.003 | 202 | 0.979 | 59 | 0.073 |
| PC C38:4 (810.8/184.1) | 65 | 0.000 | 199 | 0.917 | 49 | 0.024 |
| SM C16:0 (703.9/184.1) | 182 | 0.586 | 160 | 0.265 | 84 | 0.520 |
| SM C22:0 (787.8/184.1) | 58 | 0.000 | 126 | 0.046 | 94 | 0.854 |
| SM C24:0 (815.8/184.1) | 44 | 0.000 | 100 | 0.008 | 97 | 0.963 |
| PG C16:0/18:1 (747.6/255:8) | 75 | 0.001 | 115 | 0.023 | 61 | 0.089 |
| PG C16:0/22:6 (793.5/255.5) | 70 | 0.001 | 154 | 0.204 | 54 | 0.043 |
| PG C16:1/18:1 (745.5/281.5) | 90 | 0.003 | 82 | 0.002 | 67 | 0.154 |
| PG C16:1/20:4 (767.4/253.5) | 137 | 0.087 | 193 | 0.795 | 70 | 0.198 |
| PG C18:1/18:0 (775.6/281.0) | 28 | 0.000 | 73 | 0.001 | 51 | 0.031 |
| PG C18:1/18:1 (773.4/281.0) | 15 | 0.000 | 73 | 0.001 | 38 | 0.006 |
| PG C18:1/18:2 (771.8/281.2) | 15 | 0.000 | 69 | 0.001 | 42 | 0.010 |
| PG C18:1/20:4 (795.6/303.5) | 31 | 0.000 | 126 | 0.046 | 48 | 0.022 |
| PG C18:1/22.:5 (821.8/281.0) | 20 | 0.000 | 109 | 0.015 | 38 | 0.006 |
| PG C18:1/22:6 (819.7/281.0) | 21 | 0.000 | 138 | 0.092 | 22 | 0.000 |
| PG C18:2/22:6 (817.6/279.0) | 25 | 0.000 | 155 | 0.213 | 23 | 0.001 |
| PG C20:4/22:6 (841.5/303.5) | 30 | 0.000 | 186 | 0.659 | 26 | 0.001 |
| PG C22:5/22:5 (869.6/329.3) | 9 | 0.000 | 190 | 0.736 | 9 | 0.000 |
| PG C22:6/22:5 (867.5/329.3) | 20 | 0.000 | 200 | 0.938 | 11 | 0.000 |
| PG C22:6/22:6 (865.6/327.1) | 30 | 0.000 | 193 | 0.795 | 23 | 0.001 |
| PI C16:0/18:0 (835.4/283.2) | 147 | 0.147 | 174 | 0.452 | 73 | 0.251 |
| PI C16:0/20:4 (857.6/255.2) | 191 | 0.756 | 138 | 0.092 | 60 | 0.081 |
| PI C18:0/18:0 (865.6/283.3) | 49 | 0.000 | 139 | 0.097 | 14 | 0.000 |
| PI C18:0/18:1 (863.6/283.1) | 197 | 0.876 | 170 | 0.392 | 79 | 0.383 |
| PI C18:0/20:3 (887.6/283.1) | 185 | 0.641 | 137 | 0.087 | 65 | 0.129 |
| PI C18:0/20:4 (885.6/283.1) | 193 | 0.795 | 123 | 0.038 | 54 | 0.043 |
| PI C18:0/22:5 (911.6/283.3) | 167 | 0.351 | 144 | 0.126 | 55 | 0.048 |
| PI C18:1/18:1 (861.4/281.1) | 153 | 0.195 | 188 | 0.697 | 74 | 0.270 |
| PI C18:1/20:4 (883.6/281.2) | 201 | 0.959 | 149 | 0.162 | 68 | 0.168 |
| PS C16:0/16:0 (734.3/255.5) | 131 | 0.062 | 175 | 0.468 | 63 | 0.108 |
| PS C18:1/18:0 (788.4/283.1) | 57 | 0.000 | 103 | 0.010 | 69 | 0.183 |
| PE C18:0/20:4 (766.6/303.4) | 153 | 0.195 | 154 | 0.204 | 96 | 0.927 |
| PE C18:1/18:1 (742.6/281.1) | 151 | 0.178 | 199 | 0.917 | 70 | 0.198 |
| total Cer | 117 | 0.026 | 199 | 0.917 | 60 | 0.081 |
| TOTAL_GC | 197 | 0.876 | 103 | 0.010 | 52 | 0.035 |
| TOTAL_LC | 36 | 0.000 | 123 | 0.038 | 42 | 0.010 |
| total CTH | 43 | 0.000 | 130 | 0.058 | 40 | 0.008 |
| TOTALPC | 203 | 1.000 | 203 | 1.000 | 98 | 1.000 |
| TOTAL_SM | 72 | 0.001 | 129 | 0.055 | 97 | 0.963 |
| TOTAL_PG | 15 | 0.000 | 97 | 0.006 | 35 | 0.004 |
| TOTAL_PI | 127 | 0.049 | 137 | 0.087 | 41 | 0.009 |
| TOTAL_PE | 146 | 0.140 | 187 | 0.678 | 75 | 0.291 |
| TOTAL_PS | 59 | 0.000 | 106 | 0.012 | 70 | 0.198 |

^{a}controls n = 29
^{b}Fabrt n = 14
^{c}Fabry Het n = 14
^{d}Cer = ceramide, GC = glucosylceramide, LC = lactosylceramide, CTH = ceramide trihoxoside, SM = sphingomyelin, PC = phosphatidylcholine, PG = phosphatidylglycerol, PI = phosphatidylinositol, PS = phosphatidylserine, PE phosphatidylethanolamine
^{e}Mann-Whitney U values
^{f}significance (two-tailed)

TABLE 18
Mann-Whitney U values for lipid analyte ratios between controls^{a}, Fabry^{b} and
Fabry Hets^{c}.

| | Control v | | Control v | | Fabry v | |
|---|---|---|---|---|---|---|
| | Fabry | | Fabry Het | | Fabry Het | |
| Analyte^{d} | M-W U^{e} | Sig.^{f} | M-W U | Sig. | M-W U | Sig. |
| CTH C24:1/SM C24:0 | 18 | 0.000 | 51 | 0.000 | 39 | 0.007 |
| LC C24:1/GC C24:0 | 16 | 0.000 | 65 | 0.000 | 81 | 0.435 |
| PC C38:4/PC C32:1 | 58 | 0.000 | 187 | 0.678 | 55 | 0.048 |
| PC C36:4*PC C38:4/PC | 56 | 0.000 | 182 | 0.586 | 55 | 0.048 |
| C32:1*PC C34:1 | | | | | | |
| CTH C24:1/SM C24:0/LC | 83 | 0.002 | 191 | 0.756 | 42 | 0.010 |
| C24:1/GC C24:0 | | | | | | |
| PG C18:1/18:1/PS C18:1/18:0 | 2 | 0.000 | 35 | 0.000 | 14 | 0.000 |
| PI C18:0/18:0/PS C18:1/18:0 | 10 | 0.000 | 195 | 0.836 | 8 | 0.000 |
| PG C18:1/18:1*PI | 1 | 0.000 | 106 | 0.012 | 8 | 0.000 |
| C18:0/18:0/ | | | | | | |
| PS C18:1/18:0 | | | | | | |
| PG C18:l/18:1/SM C18:1/18:0 | 4 | 0.000 | 16 | 0.000 | 33 | 0.003 |

^{a}controls n = 29
^{b}Fabrt n = 14
^{c}Fabry Het n = 14
^{d}Cer = ceramide, GC = glucosylceramide, LC = lactosylceramide, CTH = ceramide trihoxoside, SM = sphingomyelin, PC = phosphatidylcholine, PG = phosphatidylglycerol, PI = phosphatidylinositol, PS = phosphatidylserine, PE phosphatidylethanolamine
^{e}Mann-Whitney U values
^{f}significance (two-tailed)

### Example 5

### Patient Evaluation and Monitoring of Therapy for Fabry Disease

This example provides results of studies to examine the effect of therapy on the lipid profile in plasma and urine from Fabry hemizygotes and heterozygotes.

### Materials and Methods

Plasma samples were collected from:
Control adults (19) taken from members of the Department of Genetic Medicine, Children, Youth and Women's Health Service (CYWHS), Adelaide, and control samples (19) taken from patients referred to the Department for diagnosis but were subsequently shown not to have a lysosomal storage disorder;
Fabry hemizygotes (25) and known heterozygotes (3) within Australia;
Fabry hemizygotes (5) and heterozygotes (10) who are receiving therapy in Germany.

Urine samples were collected from:
Control adults and children (28) taken from members of the Department of Genetic Medicine, CYWHS, Adelaide, and their families.
Fabry hemizygotes (13) and known heterozygotes (19) within Australia;
Fabry hemizygotes (5) and heterozygotes (10) who are receiving therapy in Germany;

Sample preparation: Lipids were extracted from plasma (100 μL) using the method of Folch and from urine (1.5 mL) using the method of Bligh/Dyer.

Mass spectrometry: A range of lipids were analysed by mass spectrometry (Tables 19 and 20) using a PE Sciex API 3000 triple-quadrupole mass spectrometer with a turbo-ionspray source and Analyst data system (PE Sciex, Concord, Ontario, Canada). Samples (20 μL) were injected into the electrospray source with a Gilson 233 autosampler using a carrying solvent of methanol at a flow rate of 80 μL/minute. For all analytes nitrogen was used as the collision gas at a pressure 2×10⁻⁵ Torr. Lipids were analysed in +ve ion mode (Cer, GC, LC, CTH, SM, PC) or −ve ion mode (gangliosides, PG, PI, PE, PS). Lipid analysis was performed using the multiple-reaction monitoring (MRM) mode. Lipid species were monitored using the ion pairs shown in Tables 2 and 3. Each ion pair was monitored for 100 milliseconds and the measurements were repeated and averaged over the injection period. Measurement of lipids was achieved by relating the peak height of each lipid ion signal to the peak height of the signal from the corresponding internal standard (Tables 19 and 20).

### Results

Table 21 shows the mean plasma concentrations of each analyte from control and Fabry hemizygotes, Fabry heterozygotes, hemizygotes on ERT and heterozygotes on ERT. Also included is the ratio of the hemizygote value over the control value, and the heterozygote value over the control value. These ratios indicate which analytes are increased in the disease state and which are decreased. Clearly, the CTH species show an increase in the hemizygote and heterozygote populations compared to the control group and this change is determined to be significant for all species in the hemizygotes by the Mann-Whitney U values shown in Table 22. Interestingly, the Mann-Witney U values for the control versus the treated hemizygotes and heterozygotes indicate that the CTH levels in the treated patients are not completely normalised. This is also evident in FIG. 23.

In addition to CTH, a number of PG species were also elevated, particularly in the heterozygotes (Table 21); these were also statistically significant based on the Mann-Whitney U values (Table 22). A number of analytes were also decreased in the hemizygote, and to a lesser extent in the heterozygote groups, compared to the control group. These include some PC species, GM3 species, as well as PI, PE and PS species (Table 21). However, most analytes showed considerable over-lap between the control and affected groups (FIG. 24).

The ability of a number of lipid ratios to distinguish between control and affected groups was also examined (Table 22) and these generally provided better discrimination than the individual lipid species. A number of lipid ratios were plotted against each other to establish whether or not there was correction in the ERT-treated patients (FIG. 25). In each plot a clear trend toward the normal lipid profile was observed for the hemizygous patients on ERT; heterozygotes were closer to normal without ERT and showed no significant change with ERT.

A similar analysis was performed on the lipid profiles observed in urine from the control and patient groups. The lipid analytes were normalised to the total level of PC to compensate for the differing levels of urinary sediment in each sample. In addition to the CTH species, significant elevations were observed in a number of other lipid types including some Cer species, LC and a number of PG species. Simultaneously, a significant decrease was observed in the level of PS 18:1/18:0 in both the hemizygote and heterozygote groups compared to the control group (Tables 23 and 24). The plasma data revealed relatively little change in CTH levels following ERT; the urine data reflected a similar pattern between treated and untreated patient groups (FIG. 26a). This trend was also borne out for most other lipid analytes (FIG. 26 and Table 24). Plotting one analyte against another (FIG. 27a) or plotting ratios of analytes (FIGS. 27b and c) improved discrimination between control and affected patient groups. In particular, the multiple ratios shown in FIG. 27c most clearly discriminated between the control and affected groups, thus demonstrating the potential of the phospholipid species in improving discrimination between Fabry hemizygotes and heterozygotes from controls.

### Discussion

Our studies on Fabry disease have demonstrated that the lipid profile in plasma and urine is significantly altered in both hemizygotes and heterozygotes. We have also shown that the altered urinary lipid profile can be used to identify heterozygotes from the control population and that the plasma lipid profile in Fabry hemizygotes is partially normalised upon enzyme replacement therapy. Thus Lipid profiling has application in the monitoring the efficacy of therapy in Fabry disease.

TABLE 19
Lipid analytes used for analysis of Fabry samples.

| | | MRM ion |
|---|---|---|
| Lipid analytes^{a} | Internal standard | pairs (m/z) |
| Cer C16:0 | Cer C17:0 | 538.7/264.4 |
| Cer C23:0 | Cer C17:0 | 636.7/264.4 |
| Cer C23:1 | Cer C17:0 | 634.7/264.4 |
| Cer C24:0 | Cer C17:0 | 650.7/264.4 |
| Cer C24:1 | Cer C17:0 | 648.7/264.4 |
| Cer C17:0 (internal standard) | | 552.7/264.4 |
| GC C16:0 | GC(d3)C16:0 | 700.6/264.4 |
| GC C22:0 | GC(d3)C16:0 | 784.7/264.4 |
| GC C24:0 | GC(d3)C16:0 | 812.7/264.4 |
| GC C24:1 | GC(d3)C16:0 | 810.8/264.4 |
| GC(d3)C16:0 (internal standard) | | 703.8/264.4 |
| LC C16:0 | LC(d3)C16:0 | 862.4/264.4 |
| LC C20:0 | LC(d3)C16:0 | 918.6/264.4 |
| LCC22:0 | LC(d3)C16:0 | 946.7/264.4 |
| LC C22:0-QH | LC(d3)C16:0 | 962.7/264.4 |
| LC C24:0 | LC(d3)C16:0 | 974.81264.4 |
| LC C24:1 | LC(d3)C16:0 | 972.8/264.4 |
| LC(d3)C16:0 (internal standard) | | 865.6/264.4 |
| CTH C16:0 | LC(d3)C16:0 | 1024.1/264.4 |
| CTH C18:0 | LC(d3)C16:0 | 1052.1/264.4 |
| CTH C20:0 | LC(d3)C16:0 | 1080.1/264.4 |
| CTH C22:0 | LC(d3)C16:0 | 1108.1/264.4 |
| CTH C24:0 | LC(d3)C16:0 | 1136.6/264.4 |
| CTH C24:1 | LC(d3)C16:0 | 1134.1/264.4 |
| SM C16:0 | PC C14:0 | 703.9/184.1 |
| SM C22:0 | PC C14:0 | 787.8/184.1 |
| SM C24:0 | PC C14:0 | 815.8/184.1 |
| PC C32:0 | PC C14:0 | 706.5/184.1 |
| PC C32:1 | PC C14:0 | 704.5/184.1 |
| PC C34:1 | PC C14:0 | 732.5/184.1 |
| PC C34:2 | PC C14:0 | 730.5/184.1 |
| PC 36:2 | PC C14:0 | 758.6/184.1 |
| PC C36:4 | PC C14:0 | 754.6/184.1 |
| PC C38:4 | PC C14:0 | 782.6/184.1 |
| PC C14:0^{b} (internal standard) | | 678.5/184.1 |

^{a}Cer = ceramide; GC = glucosylceramide; LC = lactosylceramide; CTH = ceramide trihexoside; SM = sphingomyelin; PC = phosphatidylcholine
^{b}PC C14:0 is a commercial standard and is known to have a C16:0 second fatty acid (equivalent to PC C30:0)

TABLE 20
Lipid analytes used for analysis of Fabry samples

| | | MRM ion |
|---|---|---|
| Lipid analytes^{a} | Internal standard | pairs (m/z) |
| GM3 C16:0 | GM2 C22:1 | 1151.9/290.0 |
| GM3 C22:0 | GM2 C22:1 | 1235.9/290.0 |
| GM3 C24:0 | GM2 C22:1 | 1263.1/290.0 |
| GM3 C24:1 | GM2 G22:1 | 1261.6/290.0 |
| GM2 C22:1 (Internal Standard) | | 1383.0/290.0 |
| PG 16:0/18:1 | PG 14:0/14:0 | 747.6/255.8 |
| PG 16:0/22:6 | PG 14:0/14:0 | 793.5/255.5 |
| PG 16:1/18:1 | PG 14:0/14:0 | 745.5/281.5 |
| PG 16:1/20:4 | PG 14:0/14:0 | 767.4/253.5 |
| PG 18:1/18:0 | PG 14:0/14:0 | 775.6/281.0 |
| PG 18:1/18:1 | PG 14:0/14:0 | 773.4/281.0 |
| PG 18:1/18:2 | PG 14:0/14:0 | 771.8/281.2 |
| PG 18:1/20:4 | PG 14:0/14:0 | 795.6/303.5 |
| PG 18:1/22.:5 | PG 14:0/14:0 | 821.8/281.0 |
| PG 18:1/22:6 | PG 14:0/14:0 | 819.7/281.0 |
| PG 18:2/22:6 | PG 14:0/14:0 | 817.6/279.0 |
| PG 20:4/22:6 | PG 14:0/14:0 | 841.5/303.5 |
| PG 22:5/22:5 | PG 14:0/14:0 | 869.6/329.3 |
| PG 22:6/22:5 | PG 14:0/14:0 | 867.5/329.3 |
| PG 22:6/22:6 | PG 14:0/14:0 | 865.6/327.1 |
| PG 14:0/14:0 (Internal Standard) | | 665.2/227 |
| PI 16:0/18:0 | PI 16:0/16:0 | 835.4/283.2 |
| PI 16:0/20:4 | PI 16:0/16:0 | 857.6/255.2 |
| PI 18:0/18:0 | PI 16:0/16:0 | 865.6/283.3 |
| PI 18:0/18:1 | PI 16:0/16:0 | 863.6/283.1 |
| PI 18:0/20:3 | PI 16:0/16:0 | 887.6/283.1 |
| PI 18:0/20:4 | PI 16:0/16:0 | 885.6/283.1 |
| PI 18:0/22:5 | PI 16:0/16:0 | 911.6/283.3 |
| PI 18:1/18:1 | PI 16:0/16:0 | 861.41281.1 |
| PI 18:1/20:4 | PI 16:0/16:0 | 883.6/281.2 |
| PS 16:0/16:0 | PI 16:0/16:0 | 734.3/255.5 |
| PS 18:1/18:0 | PI 16:0/16:0 | 788.41283.1 |
| PE 18:0/20:4 | PI 16:0/16:0 | 766.6/303.4 |
| PE 18:1/18:1 | PI 16:0/16:0 | 742.6/281.1 |
| PI 16:0/16:0 (Internal Standard) | | 809.5/255.1 |

^{a}GM3 = G_{M3} ganglioside; GM2 = G_{M2} ganglioside; PG = phosphatidylglycerol/lysobisphosphatidic acid; PI = phosphatidylinositol; PS = phosphatidylserine; PE = phosphatidylethanolamine.

TABLE 21
Mean lipid concentrations^{a} present in plasma from control and Fabry patients.

| | | | | Hemi | Het | | |
|---|---|---|---|---|---|---|---|
| | Control | Hemi | Het | (ERT) | (ERT) | | |
| | (n = 38) | (n = 25) | (n = 3) | (N = 5) | (N = 10) | Hemi/ | Het/ |
| Analyte | (nM) | (nM) | (nM) | (nM) | (nM) | Cont | Cont |
| Cer C16:0 (538.7/264.4) | 279 | 159 | 291 | 223 | 254 | 0.6 | 1.0 |
| Cer C20:0 (592.7/264.4) | 6 | 4 | 5 | 4 | 5 | 0.7 | 0.8 |
| Cer C20:1 (590.7/264.4) | 8 | 7 | 8 | 8 | 8 | 0.8 | 0.9 |
| Cer C23:0 (636.7/264.4) | 866 | 611 | 1046 | 688 | 844 | 0.7 | 1.2 |
| Cer C23:1 (634.8/264.4) | 55 | 38 | 62 | 42 | 47 | 0.7 | 1.1 |
| Cer C24:0 (650.7/264.4) | 3069 | 1880 | 3908 | 2272 | 2855 | 0.6 | 1.3 |
| Cer C24:1 (648.7/264.4) | 1204 | 670 | 1199 | 1123 | 1326 | 0.6 | 1.0 |
| GC C16:0 (700.6/264.4) | 793 | 714 | 941 | 857 | 1125 | 0.9 | 1.2 |
| GC C18:0 (728.6/264.4) | 123 | 126 | 145 | 145 | 180 | 1.0 | 1.2 |
| GC C20:0 (756.8/264.4) | 90 | 95 | 108 | 130 | 133 | 1.1 | 1.2 |
| GC C22:0 (784.7/264.4) | 764 | 887 | 1085 | 1187 | 1263 | 1.2 | 1.4 |
| GC C24:0 (812.7/264.4) | 1056 | 1156 | 1257 | 1544 | 1644 | 1.1 | 1.2 |
| GC C24:1 (810.8/264.4) | 833 | 783 | 762 | 1099 | 1181 | 0.9 | 0.9 |
| LC C16:0 (862.4/264.4) | 24326 | 15998 | 23618 | 21208 | 25249 | 0.7 | 1.0 |
| LC C20:0 (918.7/264.4) | 613 | 542 | 683 | 631 | 631 | 0.9 | 1.1 |
| LC C22:0 (946.7/264.4) | 2172 | 1365 | 1632 | 1617 | 1765 | 0.6 | 0.8 |
| LC C22:0-OH (962.7/264.4) | 329 | 305 | 366 | 294 | 373 | 0.9 | 1.1 |
| LC C24:0 (974.8/264.4) | 2552 | 1752 | 2200 | 2138 | 2112 | 0.7 | 0.9 |
| LC C24:1 (972.8/264.4) | 5443 | 3984 | 4571 | 5422 | 5063 | 0.7 | 0.8 |
| (LC) CTH C16:0 (1024.8/264.4) | 3752 | 9979 | 4906 | 6571 | 5300 | 2.7 | 1.3 |
| (LC) CTH C18:0 (1052.7/264.4) | 717 | 2000 | 1118 | 1543 | 962 | 2.8 | 1.6 |
| (LC) CTH C20:0 (1080.9/264.4) | 278 | 635 | 433 | 542 | 368 | 2.3 | 1.6 |
| (LC) CTH C22:0 (1108.9/264.4) | 827 | 2275 | 1174 | 1431 | 1073 | 2.7 | 1.4 |
| (LC) CTH C24:0 (1136.9/264.4) | 1031 | 3086 | 1346 | 2153 | 1339 | 3.0 | 1.3 |
| (LC) CTH C24:1 (1134.9/264.4) | 1474 | 2868 | 1684 | 2795 | 2146 | 1.9 | 1.1 |
| PC C32:0 (734.7/184.1) | 14260 | 8170 | 12840 | 11386 | 13407 | 0.6 | 0.9 |
| PC C32:1 (732.7/184.1) | 21384 | 12028 | 21104 | 19133 | 22886 | 0.6 | 1.0 |
| PC C34:1 (760.6/184.1) | 217075 | 128374 | 206524 | 185194 | 222028 | 0.6 | 1.0 |
| PC C34:2 (758.5/184.1) | 293189 | 150908 | 250741 | 240057 | 304193 | 0.5 | 0.9 |
| PC C36:2 (786.6/184.1) | 200390 | 101723 | 175606 | 159491 | 197896 | 0.5 | 0.9 |
| PC C36:4 (782.6/184.1) | 136221 | 27803 | 108696 | 100642 | 142719 | 0.2 | 0.8 |
| PC C38:4 (810.8/184.1) | 51176 | 12147 | 44030 | 41911 | 55820 | 0.2 | 0.9 |
| SM C16:0 (703.9/184.1) | 26669 | 20906 | 34769 | 25435 | 28859 | 0.8 | 1.3 |
| SM C22:0 (787.8/184.1) | 84184 | 44487 | 83880 | 64927 | 79815 | 0.5 | 1.0 |
| SM C24:0 (815.8/184.1) | 17724 | 11448 | 21421 | 15114 | 17546 | 0.6 | 1.2 |
| GM3 C16:0 (1151.9/290.0) | 9652 | 6771 | 10765 | 7858 | 8398 | 0.7 | 1.1 |
| GM3 C22:0 (1235.9/290.0) | 11 | 9 | 12 | 6 | 4 | 0.8 | 1.1 |
| GM3 C24:0 (1263.1/290.0) | 3216 | 1269 | 2318 | 1183 | 1237 | 0.4 | 0.7 |
| GM3 C24:1 (1261.6/290.0) | 4308 | 1846 | 3280 | 1620 | 1634 | 0.4 | 0.8 |
| PG 16:0/18:1 (747.6/255.8) | 2 | 2 | 2 | 2 | 2 | 1.5 | 1.3 |
| PG 16:0/22:6 (793.5/255.5) | 75 | 59 | 95 | 61 | 76 | 0.8 | 1.3 |
| PG 16:1/18:1 (745.5/281.5) | 47 | 29 | 59 | 38 | 60 | 0.6 | 1.3 |
| PG 16:1/20:4 (767.4/253.5) | 5 | 4 | 5 | 5 | 7 | 0.8 | 0.9 |
| PG 18:1/18:0 (775.6/281.0) | 46 | 42 | 76 | 42 | 56 | 0.9 | 1.7 |
| PG 18:1/18:1 (773.4/281.0) | 47 | 49 | 57 | 40 | 51 | 1.1 | 1.2 |
| PG 18:1/18:2 (771.8/281.2) | 21 | 19 | 28 | 16 | 19 | 0.9 | 1.4 |
| PG 18:1/20:4 (795.6/303.5) | 13 | 7 | 15 | 8 | 11 | 0.6 | 1.2 |
| PG 18:1/22.:5 (821.8/281.0) | 33 | 34 | 57 | 30 | 32 | 1.1 | 1.7 |
| PG 18:1/22:6 (819.7/281.0) | 45 | 53 | 107 | 68 | 60 | 1.2 | 2.3 |
| PG 18:2/22:6 (817.6/279.0) | 42 | 42 | 71 | 58 | 55 | 1.0 | 1.7 |
| PG 20:4/22:6 (841.5/303.5) | 7 | 6 | 14 | 12 | 11 | 0.9 | 1.9 |
| PG 22:5/22:5 (869.6/329.3) | 2 | 1 | 1 | 1 | 1 | 0.4 | 0.9 |
| PG 22:6/22:5 (867.5/329.3) | 1 | 1 | 2 | 2 | 1 | 0.7 | 1.5 |
| PG 22:6/22:6 (865.6/327.1) | 2 | 2 | 4 | 3 | 2 | 0.7 | 1.7 |
| PI 16:0/18:0 (835.4/283.2) | 1273 | 1912 | 2685 | 2636 | 1784 | 1.5 | 2.1 |
| PI 16:0/20:4 (857.6/255.2) | 1314 | 280 | 1175 | 1146 | 1212 | 0.2 | 0.9 |
| PI 18:0/18:0 (865.6/283.3) | 62 | 73 | 107 | 80 | 71 | 1.2 | 1.7 |
| PI 18:0/18:1 (863.6/283.1) | 1558 | 843 | 1775 | 1348 | 1321 | 0.5 | 1.1 |
| PI 18:0/20:3 (887.6/283.1) | 2753 | 791 | 2514 | 2056 | 2534 | 0.3 | 0.9 |
| PI 18:0/20:4 (885.6/283.1) | 13578 | 2908 | 11159 | 10768 | 12644 | 0.2 | 0.8 |
| PI 18:0/22:5 (911.6/283.3) | 428 | 98 | 336 | 343 | 337 | 0.2 | 0.8 |
| PI 18:1/18:1 (861.4/281.1) | 1307 | 683 | 1124 | 1038 | 991 | 0.5 | 0.9 |
| PI 18:1/20:4 (883.6/281.2) | 831 | 166 | 573 | 472 | 480 | 0.2 | 0.7 |
| PS 16:0/16:0 (734.3/255.5) | 2 | 11 | 12 | 4 | 3 | 5.6 | 6.2 |
| PS 18:1/18:0 (788.4/283.1) | 167 | 10 | 19 | 9 | 10 | 0.1 | 0.1 |
| PE 18:0/20:4 (766.6/303.4) | 279 | 36 | 204 | 202 | 420 | 0.1 | 0.7 |
| PE 18:1/18:1 (742.6/281.1) | 220 | 64 | 181 | 111 | 238 | 0.3 | 0.8 |
| Total Cer | 5487 | 3370 | 6519 | 4361 | 5339 | 0.6 | 1.2 |
| Total GC | 3658 | 3760 | 4299 | 4963 | 5526 | 1.0 | 1.2 |
| Total LC | 35435 | 23946 | 33070 | 31309 | 35192 | 0.7 | 0.9 |
| Total PC | 933695 | 441153 | 819541 | 757814 | 958949 | 0.5 | 0.9 |
| Total CTH | 8080 | 20844 | 10661 | 15035 | 11188 | 2.6 | 1.3 |
| Total GM3 | 17188 | 9894 | 16375 | 10667 | 11273 | 0.6 | 1.0 |
| Total PG | 388 | 351 | 593 | 385 | 446 | 0.9 | 1.5 |
| Total PI | 23104 | 7755 | 21449 | 19889 | 21372 | 0.3 | 0.9 |
| Total PS | 169 | 21 | 31 | 13 | 13 | 0.1 | 0.2 |
| Total PE | 498 | 100 | 385 | 314 | 658 | 0.2 | 0.8 |

^{a}Determination of lipid species was semi-quantitative (see Results and Discussion).

TABLE 22
Statistical analysis of lipid levels in plasma samples from control, Fabry hemizygotes,
Fabry heterozygotes, Fabry hemizygotes on ERT and Fabry heterozygotes on ERT.

| | Cont vs | | Cont vs | | Cont vs | | Cont vs | | Hemi vs | | Het vs | |
|---|---|---|---|---|---|---|---|---|---|---|---|---|
| | Hemi | | Het | | Hemi (ERT) | | Het (ERT) | | Hemi (ERT) | | Het (ERT) | |
| Analyte/Ratio | M-W U | Sig. | M-W U | Sig. | M-W U | Sig. | M-W U | Sig. | M-W U | Sig. | M-W U | Sig. |
| Cer C16:0 (538.7/264.4) | 71 | 0.000 | 42 | 0.453 | 50 | 0.088 | 116 | 0.060 | 21 | 0.021 | 7 | 0.176 |
| Cer C20:0 (592.7/264.4) | 293 | 0.011 | 43 | 0.483 | 60 | 0.185 | 131 | 0.134 | 54 | 0.636 | 12 | 0.612 |
| Cer C20:1 (590.7/264.4) | 354 | 0.089 | 56 | 0.960 | 91 | 0.880 | 163 | 0.493 | 43 | 0.278 | 7 | 0.176 |
| Cer C23:0 (636.7/264.4) | 177 | 0.000 | 26 | 0.121 | 63 | 0.225 | 147 | 0.275 | 40 | 0.211 | 6 | 0.128 |
| Cer C23:1 (634.8/264.4) | 170 | 0.000 | 38 | 0.342 | 52 | 0.103 | 98 | 0.020 | 39 | 0.191 | 6 | 0.128 |
| Cer C24:0 (650.7/264.4) | 134 | 0.000 | 24 | 0.099 | 46 | 0.063 | 143 | 0.233 | 31 | 0.080 | 4 | 0.063 |
| Cer C24:1 (648.7/264.4) | 120 | 0.000 | 55 | 0.920 | 86 | 0.733 | 148 | 0.286 | 15 | 0.008 | 14 | 0.866 |
| GC C16:0 (700.6/264.4) | 322 | 0.032 | 24 | 0.099 | 60 | 0.185 | 36 | 0.000 | 31 | 0.080 | 6 | 0.128 |
| GC C18:0 (728.6/264.4) | 475 | 1.000 | 31 | 0.193 | 60 | 0.185 | 50 | 0.000 | 40 | 0.211 | 7 | 0.176 |
| GC C20:0 (756.8/264.4) | 468 | 0.922 | 21 | 0.072 | 22 | 0.006 | 36 | 0.000 | 25 | 0.037 | 2 | 0.028 |
| GC C22:0 (784.7/264.4) | 371 | 0.144 | 11 | 0.021 | 20 | 0.004 | 23 | 0.000 | 26 | 0.042 | 8 | 0.237 |
| GC C24:0 (812.7/264.4) | 408 | 0.347 | 34 | 0.250 | 18 | 0.004 | 27 | 0.000 | 21 | 0.021 | 5 | 0.091 |
| GC C24:1 (810.8/264.4) | 396 | 0.267 | 48 | 0.652 | 34 | 0.021 | 68 | 0.002 | 24 | 0.032 | 4 | 0.063 |
| LC C16:0 (862.4/264.4) | 151 | 0.000 | 55 | 0.920 | 58 | 0.161 | 183 | 0.859 | 25 | 0.037 | 14 | 0.866 |
| LC C20:0 (918.7/264.4) | 397 | 0.273 | 36 | 0.293 | 75 | 0.449 | 155 | 0.374 | 35 | 0.126 | 10 | 0.398 |
| LC C22:0 (946.7/264.4) | 210 | 0.000 | 44 | 0.515 | 74 | 0.426 | 184 | 0.879 | 21 | 0.021 | 12 | 0.612 |
| LC C22:0-OH (962.7/264.4) | 471 | 0.950 | 37 | 0.317 | 89 | 0.820 | 146 | 0.264 | 56 | 0.718 | 14 | 0.866 |
| LC C24:0 (974.8/264.4) | 189 | 0.000 | 48 | 0.652 | 74 | 0.426 | 143 | 0.233 | 28 | 0.055 | 12 | 0.612 |
| LC C24:1 (972.8/264.4) | 213 | 0.000 | 42 | 0.453 | 88 | 0.791 | 158 | 0.417 | 27 | 0.048 | 14 | 0.866 |
| (LC) CTH C16:0 (1024.8/264.4) | 148 | 0.000 | 32 | 0.211 | 19 | 0.004 | 62 | 0.001 | 45 | 0.330 | 15 | 1.000 |
| (LC) CTH C18:0 (1052.7/264.4) | 103 | 0.000 | 15 | 0.035 | 12 | 0.002 | 81 | 0.006 | 55 | 0.676 | 11 | 0.499 |
| (LC) CTH C20:0 (1080.9/264.4) | 129 | 0.000 | 0 | 0.004 | 25 | 0.008 | 65 | 0.002 | 60 | 0.889 | 3 | 0.043 |
| (LC) CTH C22:0 (1108.9/264.4) | 98 | 0.000 | 13 | 0.028 | 39 | 0.034 | 105 | 0.031 | 39 | 0.191 | 9 | 0.310 |
| (LC) CTH C24:0 (1136.9/264.4) | 77 | 0.000 | 22 | 0.080 | 33 | 0.019 | 133 | 0.148 | 48 | 0.420 | 11 | 0.499 |
| (LC) CTH C24:1 (1134.9/264.4) | 170 | 0.000 | 36 | 0.293 | 20 | 0.004 | 81 | 0.006 | 53 | 0.597 | 9 | 0.310 |
| PC C32:0 (734.7/184.1) | 101 | 0.000 | 43 | 0.483 | 56 | 0.140 | 172 | 0.648 | 28 | 0.055 | 13 | 0.735 |
| PC C32:1 (732.7/184.1) | 117 | 0.000 | 53 | 0.841 | 72 | 0.384 | 172 | 0.648 | 33 | 0.101 | 12 | 0.612 |
| PC C34:1 (760.6/184.1) | 95 | 0.000 | 51 | 0.764 | 65 | 0.256 | 179 | 0.780 | 25 | 0.037 | 12 | 0.612 |
| PC C34:2 (758.5/184.1) | 64 | 0.000 | 41 | 0.423 | 40 | 0.037 | 185 | 0.899 | 16 | 0.010 | 10 | 0.398 |
| PC C36:2 (786.6/184.1) | 55 | 0.000 | 40 | 0.395 | 37 | 0.028 | 171 | 0.630 | 18 | 0.013 | 15 | 1.000 |
| PC C36:4 (782.6/184.1) | 6 | 0.000 | 46 | 0.582 | 39 | 0.034 | 177 | 0.741 | 2 | 0.001 | 11 | 0.499 |
| PC C38:4 (810.8/184.1) | 5 | 0.000 | 52 | 0.802 | 50 | 0.088 | 157 | 0.402 | 2 | 0.001 | 12 | 0.612 |
| SM C16:0 (703.9/184.1) | 202 | 0.000 | 6 | 0.011 | 91 | 0.880 | 151 | 0.322 | 36 | 0.140 | 4 | 0.063 |
| SM C22:0 (787.8/184.1) | 64 | 0.000 | 52 | 0.802 | 37 | 0.028 | 149 | 0.298 | 21 | 0.021 | 14 | 0.866 |
| SM C24:0 (815.8/184.1) | 91 | 0.000 | 31 | 0.193 | 67 | 0.289 | 172 | 0.648 | 29 | 0.062 | 9 | 0.310 |
| GM3 C16:0 (1151.9/290.0) | 257 | 0.002 | 37 | 0.317 | 72 | 0.384 | 167 | 0.559 | 44 | 0.303 | 6 | 0.128 |
| GM3 C22:0 (1235.9/290.0) | 353 | 0.087 | 54 | 0.881 | 43 | 0.049 | 27 | 0.000 | 39 | 0.191 | 4 | 0.063 |
| GM3 C24:0 (1263.1/290.0) | 34 | 0.000 | 34 | 0.250 | 2 | 0.000 | 10 | 0.000 | 58 | 0.802 | 6 | 0.128 |
| GM3 C24:1 (1261.6/290.0) | 57 | 0.000 | 36 | 0.293 | 2 | 0.000 | 10 | 0.000 | 55 | 0.676 | 7 | 0.176 |
| PG 16:0/18:1 (747.6/255.8) | 410 | 0.361 | 24 | 0.099 | 88 | 0.791 | 157 | 0.402 | 49 | 0.452 | 11 | 0.499 |
| PG 16:0/22:6 (793.5/255.5) | 293 | 0.011 | 26 | 0.121 | 63 | 0.225 | 174 | 0.685 | 52 | 0.559 | 5 | 0.091 |
| PG 16:1/18:1 (745.5/281.5) | 182 | 0.000 | 21 | 0.072 | 80 | 0.570 | 153 | 0.348 | 26 | 0.042 | 6 | 0.128 |
| PG 16:1/20:4 (767.4/253.5) | 345 | 0.068 | 56 | 0.960 | 85 | 0.705 | 188 | 0.960 | 49 | 0.452 | 15 | 1.000 |
| PG 18:1/18:0 (775.6/281.0) | 452 | 0.747 | 29 | 0.161 | 86 | 0.733 | 182 | 0.839 | 55 | 0.676 | 8 | 0.237 |
| PG 18:1/18:1 (773.4/281.0) | 433 | 0.555 | 27 | 0.133 | 73 | 0.405 | 150 | 0.310 | 51 | 0.522 | 9 | 0.310 |
| PG 18:1/18:2 (771.8/281.2) | 419 | 0.431 | 26 | 0.121 | 73 | 0.405 | 185 | 0.899 | 52 | 0.559 | 4 | 0.063 |
| PG 18:1/20:4 (795.6/303.5) | 159 | 0.000 | 34 | 0.250 | 44 | 0.053 | 167 | 0.559 | 34 | 0.113 | 9 | 0.310 |
| PG 18:1/22.:5 (821.8/281.0) | 441 | 0.633 | 25 | 0.109 | 77 | 0.495 | 165 | 0.526 | 55 | 0.676 | 6 | 0.128 |
| PG 18:1/22:6 (819.7/281.0) | 406 | 0.332 | 5 | 0.009 | 27 | 0.010 | 122 | 0.084 | 32 | 0.090 | 5 | 0.091 |
| PG 18:2/22:6 (817.6/279.0) | 447 | 0.694 | 6 | 0.011 | 49 | 0.081 | 107 | 0.035 | 22 | 0.024 | 6 | 0.128 |
| PG 20:4/22:6 (841.5/303.5) | 287 | 0.008 | 10 | 0.019 | 36 | 0.025 | 67 | 0.002 | 14 | 0.007 | 10 | 0.398 |
| PG 22:5/22:5 (869.6/329.3) | 64 | 0.000 | 42 | 0.453 | 26 | 0.009 | 82 | 0.006 | 24 | 0.032 | 11 | 0.499 |
| PG 22:6/22:5 (867.5/329.3) | 245 | 0.001 | 14 | 0.031 | 43 | 0.049 | 151 | 0.322 | 13 | 0.006 | 5 | 0.091 |
| PG 22:6/22:6 (865.6/327.1) | 241 | 0.001 | 16 | 0.040 | 50 | 0.088 | 190 | 1.000 | 11 | 0.004 | 5 | 0.091 |
| PI 16:0/18:0 (835.4/283.2) | 290 | 0.009 | 5 | 0.009 | 13 | 0.002 | 94 | 0.015 | 24 | 0.032 | 2 | 0.028 |
| PI 16:0/20:4 (857.6/255.2) | 3 | 0.000 | 52 | 0.802 | 77 | 0.495 | 174 | 0.685 | 2 | 0.001 | 13 | 0.735 |
| PI 18:0/18:0 (865.6/283.3) | 331 | 0.043 | 11 | 0.021 | 43 | 0.049 | 174 | 0.685 | 46 | 0.359 | 5 | 0.091 |
| PI 18:0/18:1 (863.6/283.1) | 139 | 0.000 | 39 | 0.368 | 82 | 0.622 | 150 | 0.310 | 21 | 0.021 | 4 | 0.063 |
| PI 18:0/20:3 (887.6/283.1) | 18 | 0.000 | 50 | 0.726 | 44 | 0.053 | 142 | 0.223 | 12 | 0.005 | 14 | 0.866 |
| PI 18:0/20:4 (885.6/283.1) | 2 | 0.000 | 41 | 0.423 | 45 | 0.058 | 125 | 0.099 | 2 | 0.001 | 15 | 1.000 |
| PI 18:0/22:5 (911.6/283.3) | 11 | 0.000 | 42 | 0.453 | 63 | 0.225 | 86 | 0.008 | 4 | 0.001 | 13 | 0.735 |
| PI 18:1/18:1 (861.4/281.1) | 174 | 0.000 | 48 | 0.652 | 78 | 0.520 | 140 | 0.204 | 22 | 0.024 | 12 | 0.612 |
| PI 18:1/20:4 (883.6/281.2) | 10 | 0.000 | 38 | 0.342 | 33 | 0.019 | 65 | 0.002 | 5 | 0.001 | 14 | 0.866 |
| PS 16:0/16:0 (734.3/255.5) | 0 | 0.000 | 6 | 0.011 | 12 | 0.002 | 80 | 0.005 | 5 | 0.001 | 9 | 0.310 |
| PS 18:1/18:0 (788.4/283.1) | 11 | 0.000 | 4 | 0.008 | 1 | 0.000 | 2 | 0.000 | 48 | 0.420 | 5 | 0.091 |
| PE 18:0/20:4 (766.6/303.4) | 12 | 0.000 | 46 | 0.582 | 79 | 0.544 | 116 | 0.060 | 0 | 0.001 | 5 | 0.091 |
| PE 18:1/18:1 (742.6/281.1) | 60 | 0.000 | 54 | 0.881 | 38 | 0.031 | 158 | 0.417 | 17 | 0.011 | 12 | 0.612 |
| Total Cer | 112 | 0.000 | 27 | 0.133 | 51 | 0.096 | 132 | 0.141 | 28 | 0.055 | 5 | 0.091 |
| TOTAL_GC | 470 | 0.944 | 29 | 0.161 | 22 | 0.006 | 32 | 0.000 | 24 | 0.032 | 6 | 0.128 |
| TOTAL_LC | 155 | 0.000 | 57 | 1.000 | 61 | 0.198 | 185 | 0.899 | 26 | 0.042 | 15 | 1.000 |
| TOTAL_PC | 44 | 0.000 | 40 | 0.395 | 42 | 0.045 | 189 | 0.980 | 15 | 0.008 | 12 | 0.612 |
| Total CTH | 132 | 0.000 | 24 | 0.099 | 21 | 0.005 | 81 | 0.006 | 49 | 0.452 | 14 | 0.866 |
| Total GM3 | 98 | 0.000 | 46 | 0.582 | 19 | 0.004 | 45 | 0.000 | 50 | 0.487 | 6 | 0.128 |
| TOTAL_PG | 343 | 0.064 | 7 | 0.012 | 93 | 0.940 | 183 | 0.859 | 39 | 0.191 | 7 | 0.176 |
| TOTAL_PI | 16 | 0.000 | 52 | 0.802 | 60 | 0.185 | 125 | 0.099 | 5 | 0.001 | 13 | 0.735 |
| TOTAL_PS | 40 | 0.000 | 14 | 0.031 | 1 | 0.000 | 3 | 0.000 | 27 | 0.048 | 5 | 0.091 |
| TOTAL_PE | 11 | 0.000 | 47 | 0.617 | 48 | 0.075 | 124 | 0.094 | 0 | 0.001 | 7 | 0.176 |
| CTH 16:0/PC 36:4 | 0 | 0.000 | 10 | 0.019 | 18 | 0.004 | 110 | 0.042 | 1 | 0.001 | 7 | 0.176 |
| PG 18:1_22:6/PI 18:0_20:4 | 2 | 0.000 | 11 | 0.021 | 12 | 0.002 | 81 | 0.006 | 6 | 0.002 | 7 | 0.176 |
| PI 16:0_18:0/PI 18:0_20:4 | 11 | 0.000 | 9 | 0.016 | 3 | 0.000 | 64 | 0.001 | 15 | 0.008 | 8 | 0.237 |
| PI 16:0_18:0/PS 18:1_18:0 | 14 | 0.000 | 0 | 0.004 | 0 | 0.000 | 1 | 0.000 | 34 | 0.113 | 12 | 0.612 |
| PS 16:0_16:0/PS 18:1_18:0 | 0 | 0.000 | 0 | 0.004 | 0 | 0.000 | 1 | 0.000 | 11 | 0.004 | 14 | 0.866 |
| PG 18:1_22:6/PG 22:5_22:5 | 11 | 0.000 | 7 | 0.012 | 3 | 0.000 | 47 | 0.000 | 38 | 0.173 | 14 | 0.866 |

Control (n = 38); Hemi (n = 25); Het (n = 3); Hemi (ERT) (n = 5); Het (ERT) (N = 10)

TABLE 23
Mean lipid concentrations^{a} present in urine from control and Fabry patients.

| | | | | Hemi | Het | | |
|---|---|---|---|---|---|---|---|
| | Control | Hemi | Het | (ERT) | (ERT) | Hemi/ | Het/ |
| Analyte | (n = 28) | (n = 13) | (n = 19) | (n = 5) | (n = 10) | Cont | Cont |
| Cer C16:0 (538.7/264.4) | 20 | 31 | 37 | 55 | 24 | 1.6 | 1.9 |
| Cer C24:0 (650.7/264.4) | 12 | 18 | 16 | 40 | 11 | 1.5 | 1.3 |
| Cer C24:1 (648.7/264.4) | 5 | 14 | 11 | 21 | 5 | 2.7 | 2.2 |
| Cer C20:0 (592.7/264.4) | 2 | 2 | 2 | 8 | 1 | 1.0 | 0.8 |
| Cer C20:1 (590.7/264.4) | 3 | 2 | 12 | 15 | 3 | 0.9 | 4.3 |
| Cer C23:0 (636.7/264.4) | 5 | 5 | 23 | 37 | 4 | 1.1 | 4.6 |
| Cer C23:1 (634.8/264.4) | 4 | 5 | 8 | 20 | 5 | 1.2 | 2.0 |
| GC C16:0 (700.6/264.4) | 28 | 25 | 25 | 73 | 22 | 0.9 | 0.9 |
| GC C22:0 (784.7/264.4) | 38 | 32 | 23 | 75 | 21 | 0.8 | 0.6 |
| GC C24:0 (812.7/264.4) | 34 | 30 | 25 | 66 | 22 | 0.9 | 0.7 |
| GC C24:1 (810.8/264.4) | 12 | 14 | 10 | 45 | 9 | 1.2 | 0.8 |
| LC C16:0 (862.4/264.4) | 158 | 386 | 336 | 556 | 451 | 2.4 | 2.1 |
| LC C20:0 (918.7/264.4) | 118 | 317 | 138 | 614 | 185 | 2.7 | 1.2 |
| LC C22:0 (946.7/264.4) | 111 | 406 | 178 | 682 | 263 | 3.7 | 1.6 |
| LC C22:0-OH (962.7/264.4) | 147 | 681 | 203 | 843 | 334 | 4.6 | 1.4 |
| LC C24:0 (974.8/264.4) | 94 | 727 | 176 | 529 | 255 | 7.7 | 1.9 |
| LC C24:1 (972.8/264.4) | 86 | 311 | 202 | 498 | 238 | 3.6 | 2.4 |
| (LC) CTH C16:0 | 70 | 998 | 151 | 1288 | 293 | 14.4 | 2.2 |
| (1024.8/264.4) | | | | | | | |
| (LC) CTH C18:0 | 46 | 505 | 83 | 520 | 97 | 11.0 | 1.8 |
| (1052.7/264.4) | | | | | | | |
| (LC) CTH C20:0 | 186 | 817 | 162 | 1008 | 194 | 4.4 | 0.9 |
| (1080.9/264.4) | | | | | | | |
| (LC) CTH C22:0 | 75 | 1964 | 213 | 1791 | 350 | 26.1 | 2.8 |
| (1108.9/264.4) | | | | | | | |
| (LC) CTH C24:0 | 74 | 2669 | 178 | 2361 | 486 | 35.9 | 2.4 |
| (1136.9/264.4) | | | | | | | |
| (LC) CTH C24:1 | 85 | 2124 | 237 | 1586 | 389 | 25.0 | 2.8 |
| (1134.9/264.4) | | | | | | | |
| PC C32:0 (734.7/184.1) | 57 | 44 | 51 | 57 | 46 | 0.8 | 0.9 |
| PC C32:1 (732.7/184.1) | 57 | 40 | 54 | 54 | 47 | 0.7 | 1.0 |
| PC C34:1 (760.6/184.1) | 397 | 353 | 344 | 382 | 338 | 0.9 | 0.9 |
| PC C34:2 (758.5/184.1) | 219 | 261 | 189 | 201 | 241 | 1.2 | 0.9 |
| PC C36:2 (786.6/184.1) | 163 | 171 | 195 | 193 | 184 | 1.0 | 1.2 |
| PC C36:4 (782.6/184.1) | 78 | 95 | 123 | 81 | 106 | 1.2 | 1.6 |
| PC C38:4 (810.8/184.1) | 30 | 37 | 44 | 31 | 39 | 1.2 | 1.5 |
| SM C16:0 (703.9/184.1) | 209 | 199 | 261 | 246 | 175 | 1.0 | 1.2 |
| SM C22:0 (787.8/184.1) | 293 | 215 | 267 | 249 | 175 | 0.7 | 0.9 |
| SM C24:0 (815.8/184.1) | 245 | 161 | 175 | 196 | 132 | 0.7 | 0.7 |
| PG 16:0/18:1 (747.6/255.8) | 0 | 2 | 1 | 1 | 1 | 4.8 | 2.5 |
| PG 16:0/22:6 (793.5/255.5) | 2 | 12 | 4 | 10 | 3 | 4.9 | 1.7 |
| PG 16:1/18:1 (745.5/281.5) | 2 | 7 | 9 | 5 | 3 | 3.6 | 5.0 |
| PG 16:1/20:4 (767.4/253.5) | 1 | 1 | 1 | 1 | 0 | 1.1 | 1.5 |
| PG 18:1/18:0 (775.6/281.0) | 5 | 43 | 20 | 24 | 16 | 8.5 | 3.9 |
| PG 18:1/18:1 (773.4/281.0) | 21 | 264 | 63 | 132 | 80 | 12.7 | 3.0 |
| PG 18:1/18:2 (771.8/281.2) | 5 | 83 | 17 | 37 | 24 | 15.2 | 3.1 |
| PG 18:1/20:4 (795.6/303.5) | 1 | 6 | 4 | 6 | 3 | 7.5 | 4.6 |
| PG 18:1/22.:5 (821.8/281.0) | 3 | 24 | 7 | 12 | 7 | 9.6 | 2.9 |
| PG 18:1/22:6 (819.7/281.0) | 10 | 93 | 16 | 68 | 25 | 9.3 | 1.6 |
| PG 18:2/22:6 (817.6/279.0) | 5 | 40 | 8 | 36 | 13 | 7.4 | 1.5 |
| PG 20:4/22:6 (841.5/303.5) | 1 | 3 | 1 | 4 | 1 | 4.1 | 1.3 |
| PG 22:5/22:5 (869.6/329.3) | 0 | 3 | 1 | 1 | 1 | 6.7 | 1.3 |
| PG 22:6/22:5 (867.5/329.3) | 1 | 6 | 2 | 7 | 3 | 4.8 | 1.2 |
| PG 22:6/22:6 (865.6/327.1) | 4 | 18 | 4 | 23 | 7 | 4.8 | 1.1 |
| PI 16:0/18:0 (835.4/283.2) | 33 | 27 | 23 | 30 | 18 | 0.8 | 0.7 |
| PI 16:0/20:4 (857.6/255.2) | 13 | 11 | 10 | 11 | 5 | 0.9 | 0.8 |
| PI 18:0/18:0 (865.6/283.3) | 19 | 82 | 14 | 103 | 20 | 4.4 | 0.7 |
| PI 18:0/18:1 (863.6/283.1) | 17 | 16 | 35 | 17 | 8 | 0.9 | 2.0 |
| PI 18:0/20:3 (887.6/283.1) | 19 | 18 | 30 | 15 | 8 | 0.9 | 1.6 |
| PI 18:0/20:4 (885.6/283.1) | 64 | 61 | 102 | 52 | 32 | 1.0 | 1.6 |
| PI 18:0/22:5 (911.6/283.3) | 4 | 4 | 6 | 4 | 2 | 1.0 | 1.6 |
| PI 18:1/18:1 (861.4/281.1) | 9 | 9 | 69 | 8 | 4 | 1.0 | 7.8 |
| PI 18:1/20:4 (883.6/281.2) | 7 | 6 | 10 | 6 | 3 | 0.9 | 1.4 |
| PS 16:0/16:0 (734.3/255.5) | 1 | 1 | 62 | 3 | 43 | 1.2 | 69.5 |
| PS 18:1/18:0 (788.4/283.1) | 72 | 38 | 50 | 40 | 30 | 0.5 | 0.7 |
| PE 18:0/20:4 (766.6/303.4) | 3 | 3 | 3 | 3 | 2 | 0.8 | 1.0 |
| PE 18:1/18:1 (742.6/281.1) | 8 | 6 | 14 | 8 | 5 | 0.7 | 1.7 |
| total Cer | 51 | 78 | 110 | 196 | 53 | 1.5 | 2.2 |
| total GC | 112 | 101 | 83 | 259 | 74 | 0.9 | 0.7 |
| total LC | 714 | 2829 | 1232 | 3722 | 1726 | 4.0 | 1.7 |
| total CTH | 536 | 9078 | 1024 | 8554 | 1808 | 17.0 | 1.9 |
| total PC | 1000 | 1000 | 1000 | 1000 | 1000 | 1.0 | 1.0 |
| total SM | 748 | 576 | 703 | 691 | 481 | 0.8 | 0.9 |
| total PG | 61 | 604 | 156 | 366 | 187 | 9.8 | 2.5 |
| total PI | 152 | 208 | 276 | 215 | 80 | 1.4 | 1.8 |
| total PE | 11 | 9 | 17 | 12 | 7 | 0.8 | 1.5 |
| total PS | 73 | 39 | 111 | 42 | 73 | 0.5 | 1.5 |

*Determination of lipid species was semi-quantitative (see Results and Discussion). Results are expressed as pmol/nmol total PC

TABLE 24
Statistical analysis of lipid levels in urine samples from control, Fabry hemizygotes,
Fabry heterozygotes, Fabry hemizygotes on ERT and Fabry heterozygotes on ERT.

| | Cont vs | | Cont vs | | Cont vs | | Cont vs | | Hemi vs | | Het vs | |
|---|---|---|---|---|---|---|---|---|---|---|---|---|
| | Hemi | | Het | | Hemi (ERTI) | | Het (ERT) | | Hemi (ERT) | | Het (ERT) | |
| Analyte | M-W U | Sig. | M-W U | Sig. | M-W U | Sig. | M-W U | Sig. | M-W U | Sig. | M-W U | Sig. |
| Cer C16:0 (538.7/264.4) | 110 | 0.044 | 193 | 0.114 | 11 | 0.003 | 108 | 0.289 | 14 | 0.068 | 88 | 0.748 |
| Cer C24:0 (650.7/264.4) | 111 | 0.047 | 236 | 0.515 | 9 | 0.002 | 140 | 1.000 | 9 | 0.021 | 82 | 0.551 |
| Cer C24:1 (648.7/264.4) | 70 | 0.002 | 239 | 0.558 | 8 | 0.002 | 133 | 0.817 | 18 | 0.153 | 87 | 0.714 |
| Cer C20:0 (592.7/264.4) | 151 | 0.385 | 232 | 0.461 | 45 | 0.209 | 108 | 0.289 | 28 | 0.657 | 83 | 0.582 |
| Cer C20:1 (590.7/264.4) | 181 | 0.978 | 178 | 0.056 | 60 | 0.616 | 135 | 0.868 | 27 | 0.588 | 58 | 0.090 |
| Cer C23:0 (636.7/264.4) | 132 | 0.161 | 241 | 0.588 | 32 | 0.056 | 114 | 0.389 | 24 | 0.402 | 93 | 0.927 |
| Cer C23:1 (634.8/264.4) | 118 | 0.073 | 219 | 0.308 | 48 | 0.269 | 121 | 0.529 | 31 | 0.882 | 68 | 0.215 |
| GC C16:0 (700.6/264.4) | 161 | 0.556 | 237 | 0.530 | 35 | 0.079 | 112 | 0.353 | 14 | 0.068 | 87 | 0.714 |
| GC C22:0 (784.7/264.4) | 129 | 0.138 | 113 | 0.001 | 43 | 0.175 | 43 | 0.001 | 14 | 0.068 | 90 | 0.819 |
| GC C24:0 (812.7/264.4) | 161 | 0.556 | 163 | 0.026 | 37 | 0.098 | 63 | 0.011 | 13 | 0.055 | 94 | 0.963 |
| GC C24:1 (810.8/264.4) | 143 | 0.275 | 199 | 0.146 | 36 | 0.088 | 92 | 0.112 | 23 | 0.349 | 94 | 0.963 |
| LC C16:0 (862.4/264.4) | 101 | 0.023 | 131 | 0.003 | 7 | 0.002 | 52 | 0.004 | 22 | 0.301 | 85 | 0.646 |
| LC C20:0 (918.7/264.4) | 47 | 0.000 | 247 | 0.680 | 9 | 0.002 | 112 | 0.353 | 22 | 0.301 | 80 | 0.491 |
| LC C22:0 (946.7/264.4) | 49 | 0.000 | 175 | 0.049 | 2 | 0.001 | 71 | 0.022 | 19 | 0.183 | 82 | 0.551 |
| LC C22:0-OH (962.7/264.4) | 58 | 0.001 | 212 | 0.242 | 6 | 0.001 | 110 | 0.320 | 20 | 0.218 | 85 | 0.646 |
| LC C24:0 (974.8/264.4) | 9 | 0.000 | 123 | 0.002 | 1 | 0.001 | 63 | 0.011 | 29 | 0.730 | 82 | 0.551 |
| LC C24:1 (972.8/264.4) | 43 | 0.000 | 105 | 0.000 | 3 | 0.001 | 47 | 0.002 | 23 | 0.349 | 91 | 0.854 |
| (LC) CTH C16:0 (1024.8/264.4) | 35 | 0.000 | 160 | 0.022 | 3 | 0.001 | 57 | 0.006 | 17 | 0.127 | 82 | 0.551 |
| (LC) CTH C18:0 (1052.7/264.4) | 21 | 0.000 | 193 | 0.114 | 17 | 0.008 | 112 | 0.353 | 32 | 0.961 | 92 | 0.891 |
| (LC) CTH C20:0 (1080.9/264.4) | 67 | 0.001 | 244 | 0.633 | 15 | 0.006 | 134 | 0.842 | 20 | 0.218 | 92 | 0.891 |
| (LC) CTH C22:0 (1108.9/264.4) | 39 | 0.000 | 134 | 0.004 | 6 | 0.001 | 92 | 0.112 | 30 | 0.805 | 85 | 0.646 |
| (LC) CTH C24:0 (1136.9/264.4) | 23 | 0.000 | 138 | 0.006 | 4 | 0.001 | 70 | 0.020 | 30 | 0.805 | 94 | 0.963 |
| (LC) CTH C24:1 (1134.9/264.4) | 42 | 0.000 | 126 | 0.002 | 3 | 0.001 | 69 | 0.019 | 32 | 0.961 | 94 | 0.963 |
| PC C32:0 (734.7/184.1) | 98 | 0.019 | 239 | 0.558 | 68 | 0.920 | 106 | 0.260 | 10 | 0.027 | 84 | 0.614 |
| PC C32:1 (732.7/184.1) | 51 | 0.000 | 263 | 0.948 | 55 | 0.451 | 81 | 0.050 | 7 | 0.012 | 62 | 0.130 |
| PC C34:1 (760.6/184.1) | 97 | 0.017 | 145 | 0.009 | 55 | 0.451 | 51 | 0.003 | 22 | 0.301 | 77 | 0.409 |
| PC C34:2 (758.5/184.1) | 74 | 0.002 | 211 | 0.233 | 58 | 0.547 | 98 | 0.164 | 7 | 0.012 | 52 | 0.048 |
| PC C36:2 (786.6/184.1) | 133 | 0.170 | 145 | 0.009 | 68 | 0.920 | 72 | 0.024 | 19 | 0.183 | 74 | 0.335 |
| PC C36:4 (782.6/184.1) | 82 | 0.005 | 218 | 0.298 | 49 | 0.292 | 61 | 0.009 | 21 | 0.257 | 43 | 0.017 |
| PC C38:4 (810.8/184.1) | 60 | 0.001 | 210 | 0.225 | 42 | 0.160 | 70 | 0.020 | 22 | 0.301 | 42 | 0.015 |
| SM C16:0 (703.9/184.1) | 168 | 0.695 | 240 | 0.573 | 40 | 0.132 | 94 | 0.127 | 18 | 0.153 | 76 | 0.383 |
| SM C22:0 (787.8/184.1) | 71 | 0.002 | 137 | 0.005 | 49 | 0.292 | 21 | 0.000 | 17 | 0.127 | 69 | 0.233 |
| SM C24:0 (815.8/184.1) | 57 | 0.000 | 118 | 0.001 | 42 | 0.160 | 26 | 0.000 | 18 | 0.153 | 81 | 0.521 |
| PG 16:0/18:1 (747.6/255.8) | 67 | 0.001 | 118 | 0.001 | 33 | 0.063 | 102 | 0.208 | 24 | 0.402 | 77 | 0.409 |
| PG 16:0/22:6 (793.5/2555) | 56 | 0.000 | 192 | 0.109 | 37 | 0.098 | 134 | 0.842 | 24 | 0.402 | 79 | 0.463 |
| PG 16:1/18:1 (745.5/281.5) | 68 | 0.001 | 84 | 0.000 | 33 | 0.063 | 117 | 0.446 | 25 | 0.460 | 77 | 0.409 |
| PG 16:1/20:4 (767.4/253.5) | 132 | 0.161 | 252 | 0.762 | 52 | 0.366 | 102 | 0.208 | 25 | 0.460 | 72 | 0.291 |
| PG 18:1/18:0 (775.6/281.0) | 23 | 0.000 | 89 | 0.000 | 21 | 0.014 | 103 | 0.220 | 23 | 0.349 | 81 | 0.521 |
| PG 18:1/18:1 (773.4/281.0) | 12 | 0.000 | 96 | 0.000 | 10 | 0.003 | 96 | 0.145 | 21 | 0.257 | 86 | 0.680 |
| PG 18:1/18:2 (771.8/281.2) | 4 | 0.000 | 83 | 0.000 | 12 | 0.004 | 82 | 0.055 | 17 | 0.127 | 88 | 0.748 |
| PG 18:1/20:4 (795.6/303.5) | 20 | 0.000 | 168 | 0.034 | 20 | 0.012 | 110 | 0.320 | 31 | 0.882 | 93 | 0.927 |
| PG 18:1/22.:5 (821.8/281.0) | 10 | 0.000 | 154 | 0.015 | 27 | 0.031 | 112 | 0.353 | 21 | 0.257 | 90 | 0.819 |
| PG 18:1/22:6 (819.7/281.0) | 24 | 0.000 | 188 | 0.091 | 24 | 0.021 | 105 | 0.246 | 19 | 0.183 | 88 | 0.748 |
| PG 18:2/22:6 (817.6/279.0) | 23 | 0.000 | 201 | 0.159 | 24 | 0.021 | 104 | 0.233 | 21 | 0.257 | 88 | 0.748 |
| PG 20:4/22:6 (841.5/303.5) | 26 | 0.000 | 242 | 0.603 | 32 | 0.056 | 113 | 0.371 | 29 | 0.730 | 89 | 0.783 |
| PG 22:5/22:5 (869.6/329.3) | 25 | 0.000 | 245 | 0.649 | 42 | 0.160 | 127 | 0.667 | 24 | 0.402 | 92 | 0.891 |
| PG 22:6/22:5 (867.5/329.3) | 31 | 0.000 | 259 | 0.879 | 38 | 0.108 | 106 | 0.260 | 20 | 0.218 | 78 | 0.435 |
| PG 22:6/22:6 (865.6/327.1) | 36 | 0.000 | 252 | 0.762 | 41 | 0.145 | 117 | 0.446 | 20 | 0.218 | 87 | 0.714 |
| PI 16:0/18:0 (835.4/283.2) | 134 | 0.179 | 252 | 0.762 | 51 | 0.340 | 84 | 0.063 | 27 | 0.588 | 68 | 0.215 |
| PI 16:0/20:4 (857.6/255.2) | 159 | 0.519 | 172 | 0.042 | 63 | 0.725 | 37 | 0.001 | 31 | 0.882 | 71 | 0.271 |
| PI 18:0/18:0 (865.6/283.3) | 53 | 0.000 | 197 | 0.135 | 35 | 0.079 | 124 | 0.596 | 28 | 0.657 | 84 | 0.614 |
| PI 18:0/18:1 (863.6/283.1) | 178 | 0.911 | 211 | 0.233 | 63 | 0.725 | 23 | 0.000 | 24 | 0.402 | 25 | 0.001 |
| PI 18:0/20:3 (887.6/283.1) | 175 | 0.845 | 161 | 0.023 | 57 | 0.514 | 24 | 0.000 | 28 | 0.657 | 51 | 0.044 |
| PI 18:0/20:4 (885.6/283.1) | 169 | 0.716 | 144 | 0.008 | 58 | 0.547 | 36 | 0.001 | 26 | 0.522 | 74 | 0.335 |
| PI 18:0/22:5 (911.6/283.3) | 160 | 0.538 | 185 | 0.079 | 65 | 0.802 | 49 | 0.003 | 28 | 0.657 | 70 | 0.251 |
| PI 18:1/18:1 (861.4/281.1) | 137 | 0.207 | 226 | 0.386 | 59 | 0.581 | 46 | 0.002 | 28 | 0.657 | 50 | 0.039 |
| PI 18:1/20:4 (883.6/281.2) | 169 | 0.716 | 183 | 0.072 | 65 | 0.802 | 24 | 0.000 | 29 | 0.730 | 46 | 0.025 |
| PS 16:0/16:0 (734.3/255.5) | 122 | 0.093 | 234 | 0.488 | 58 | 0.547 | 125 | 0.619 | 29 | 0.730 | 93 | 0.927 |
| PS 18:1/18:0 (788.4/283.1) | 71 | 0.002 | 126 | 0.002 | 37 | 0.098 | 33 | 0.000 | 29 | 0.730 | 69 | 0.233 |
| PE 18:0/20:4 (766.6/303.4) | 149 | 0.355 | 184 | 0.075 | 67 | 0.880 | 92 | 0.112 | 29 | 0.730 | 94 | 0.963 |
| PE 18:1/18:1 (742.6/281.1) | 139 | 0.228 | 257 | 0.845 | 66 | 0.841 | 70 | 0.020 | 26 | 0.522 | 48 | 0.031 |
| total Cer | 109 | 0.041 | 204 | 0.179 | 7 | 0.002 | 135 | 0.868 | 13 | 0.055 | 76 | 0.383 |
| total GC | 165 | 0.634 | 164 | 0.027 | 36 | 0.088 | 63 | 0.011 | 13 | 0.055 | 89 | 0.783 |
| total LC | 29 | 0.000 | 139 | 0.006 | 4 | 0.001 | 56 | 0.005 | 25 | 0.460 | 85 | 0.646 |
| total CTH | 38 | 0.000 | 159 | 0.020 | 4 | 0.001 | 90 | 0.097 | 32 | 0.961 | 95 | 1.000 |
| total PC | 182 | 1.000 | 266 | 1.000 | 70 | 1.000 | 140 | 1.000 | 32.5 | 1.000 | 95 | 1.000 |
| total SM | 81 | 0.005 | 170 | 0.037 | 56 | 0.482 | 36 | 0.001 | 16 | 0.104 | 78 | 0.435 |
| total PG | 13 | 0.000 | 129 | 0.003 | 19 | 0.010 | 106 | 0.260 | 22 | 0.301 | 87 | 0.714 |
| total PI | 103 | 0.027 | 166 | 0.030 | 54 | 0.422 | 46 | 0.002 | 27 | 0.588 | 67 | 0.199 |
| total PE | 139 | 0.228 | 242 | 0.603 | 63 | 0.725 | 78 | 0.040 | 27 | 0.588 | 58 | 0.090 |
| total PS | 73 | 0.002 | 152 | 0.013 | 39 | 0.120 | 56 | 0.005 | 29 | 0.730 | 72 | 0.291 |
| CTH24:0/SM24:0 | 11 | 0.000 | 88 | 0.000 | 2 | 0.001 | 9 | 0.000 | 25 | 0.460 | 82 | 0.551 |
| Cer24:1/GC22:0 | 42 | 0.000 | 76 | 0.000 | 13 | 0.004 | 23 | 0.000 | 29 | 0.730 | 93 | 0.927 |
| LC24:0/GC22:0 | 1 | 0.000 | 49 | 0.000 | 0 | 0.000 | 0 | 0.000 | 12 | 0.043 | 64 | 0.155 |
| PG18:1/18:1/SM24:0 | 4 | 0.000 | 22 | 0.000 | 12 | 0.004 | 43 | 0.001 | 20 | 0.218 | 83 | 0.582 |
| PG18:1/18:1/PS 18:1/18:0 | 2 | 0.000 | 34 | 0.000 | 1 | 0.001 | 7 | 0.000 | 23 | 0.349 | 75 | 0.359 |
| PI18:0/18:0/PS 18:1/18:0 | 29 | 0.000 | 219 | 0.308 | 27 | 0.031 | 35 | 0.001 | 29 | 0.730 | 37 | 0.008 |
| PC38:4/PC32:1 | 55 | 0.000 | 243 | 0.618 | 43 | 0.175 | 47 | 0.002 | 16 | 0.104 | 39 | 0.010 |
| CTH24:0*PG18:1/18:1/SM24:0 | 6 | 0.000 | 65 | 0.000 | 1 | 0.001 | 40 | 0.001 | 27 | 0.588 | 92 | 0.891 |
| PG18:1/18:1*PI18:0/18:0/PS 18:1/18:0 | 13 | 0.000 | 117 | 0.001 | 2 | 0.001 | 37 | 0.001 | 26 | 0.522 | 80 | 0.491 |
| CTH24:0*LC24:0/GC22:0/SM24:0 | 3 | 0.000 | 65 | 0.000 | 0 | 0.000 | 1 | 0.000 | 20 | 0.218 | 71 | 0.271 |
| CTH24:0*LC24:0*Cer24:1/GC22:0/ | 9 | 0.000 | 50 | 0.000 | 0 | 0.000 | 7 | 0.000 | 26 | 0.522 | 81 | 0.521 |
| SM24:0 | | | | | | | | | | | | |
| PC38:4*PG18:1/18:1*PI18:0/18:0/ | | | | | | | | | | | | |
| PC32:1/PS 18:1/18:0 | 4 | 0.000 | 134 | 0.004 | 2 | 0.001 | 30 | 0.000 | 24 | 0.402 | 69 | 0.233 |
| CTH24:0*LC24:0/PS18:1/18:0 | 9 | 0.000 | 90 | 0.000 | 0 | 0.000 | 8 | 0.000 | 32 | 0.961 | 75 | 0.359 |
| PG18:1/18:1/GC22:0/SM24:0 | 2 | 0.000 | 12 | 0.000 | 28 | 0.035 | 29 | 0.000 | 12 | 0.043 | 86 | 0.680 |
| PG18:1/18:2/GC22:0/SM24:0 | 0 | 0.000 | 7 | 0.000 | 50 | 0.315 | 25 | 0.000 | 12 | 0.043 | 78 | 0.435 |
| CTH22:0*LC24:0/PS18:1/18:0 | 7 | 0.000 | 67 | 0.000 | 1 | 0.001 | 13 | 0.000 | 31 | 0.882 | 81 | 0.521 |

Control (n = 28); Hemi (n = 13); Het (n — 19); Hemi (ERT) (n = 5); Het (ERT) (N = 10)

### REFERENCES

1. Meikle, P. J., Hopwood, J. J., Clague, A. E. and Carey, W. F., Prevalence of lysosomal storage disorders. Jama. 1999, 281: 249-254.
2. Rider, J. A. and Rider, D. L., Thirty years of Batten disease research: present status and future goals. Mol. Genet. Metab. 1999, 66: 231-233.
3. Santavuori, P., Neuronal ceroid-lipofuscinoses in childhood. Brain Dev. 1988, 10: 80-83.
4. Conzelmann, E. and Sandhoff, K, Partial enzyme deficiencies: residual activities and the development of neurological disorders. Dev. Neurosci. 1983, 6: 58-71.
5. Leinekugel, P., Michel, S., Conzelmann, E. and Sandhoff, K., Quantitative correlation between the residual activity of beta-hexosaminidase A and arylsulfatase A and the severity of the resulting lysosomal storage disease. Hum. Genet. 1992, 88: 513-523.
6. Carpenter, K. H. and Wiley, V., Application of tandem mass spectrometry to biochemical genetics and newborn screening. Clin. Chim. Acta. 2002, 322: 1-10.
7. Chace, D. H., Kalas, T. A. and Naylor, E. W., The application of tandem mass spectrometry to neonatal screening for inherited disorders of intermediary metabolism. Annu. Rev. Genomics Hum. Genet. 2002, 3: 17-45.

1. A method of assessing an LSD (Lysosomal storage disorder) status of an individual the method comprising the steps of,
taking a tissue or body fluid sample from the individual,
estimating a level in the sample of each of three or more compound indicators, said indicators being indicative of the level of respectively each of three or more lipid containing storage associated compounds,
calculating an LSD index number using all of said compound indicators,
and comparing the LSD index number of the sample with a standard to provide an assessment of the LSD status of the individual.

2. A method of assessing an LSD status of an individual the method comprising the steps of,
taking a tissue or body fluid sample from the individual,
estimating a level in the sample of each of two or more compound indicators being indicative of the level respectively of each of two or more lipid containing storage associated compounds,
calculating an LSD index number using all of said compound indicators,
and comparing the LSD index number of the sample with a standard to provide an assessment of the LSD status of the individual,
the two or more storage associated compounds selected to discriminate between an LSD individual from a non-LSD individual with an acceptable confidence level.

3. A method for screening for the status of two or more LSDs in an individual,
taking a single tissue or body fluid sample from the individual,
estimating a level in the sample of each three or more compound indicators being indicative of the concentration respectively of each of three or more lipid containing storage associated compounds,
calculating a first LSD index number using a first set of two or more of said compound indicators and comparing the first LSD index number of the sample with a first control indicator to provide an assessment of the LSD status of the first LSD,
and calculating a second LSD index number using a second set of two or more of said compound indicators and comparing the second LSD index number of the individual with a second standard to provide an assessment of the LSD status of the second LSD in the individual.

4. The method as in any one of claims 1 to 3 wherein the storage associated compounds are selected from the group of compounds consisting of phospholipids and glycolipids.

5. The method of claim 4 wherein the glycolipids are selected from the group comprising glycerolipids, glycoposhatidylinositols, glycosphingolipids.

6. The method of claim 4 wherein the storage associated compounds are phospholipids and are characterised by head groups selected from the group consisting of phosphatidyl serine, phosphatidylinositol, phosphatidyl ethanolamine and sphingomyelin phosphatidyl glycerol, phosphatidyl serine, phosphatidyl inositol, phosophatidyl ethanolamine, cerebroside or a ganglioside.

7. The method of claim 6 wherein the phospholipids are further characterised by the fatty acids which are selected from the group consisting of 1-palmitoyl-2-oleoyl-, 1-palmitoyl-2-linoleoyl-, 1-palmitoly-2-arachadonyl-, 1-palmitoyl-2-docosahexanoyl.

8. The method of claim 4 wherein the indicator of the level of lipid containing storage associated compound is measured by a technique selected from the group consisting of electrophoresis, chromatography, Gas chromatography, HPLC (High pressure Liquid Chromatography), Nuclear Magnetic resonance analysis, gas chromatography-mass spectrometry (GC-MS), GC linked to Fourier-transform infrared spectroscopy (FTIR), and silver ion and reversed-phase high-performance liquid chromatography (HPLC) and mass spectrometry.

9. The method of claim 8 wherein the technique is mass spectrometry.

10. The method of claim 9 where the mass spectrometry is electrospray ionisation-tandem mass spectrometry (ESI-MSMS).

11. The method as in claim 4 wherein at least two lipid containing storage associated compounds are selected one from a first group that increases in LSD individual and a second from a second group that decreases in levels in LSD individual and the values for the first and second compounds are combined to give an index number.

12. The method as in claim 4 wherein the is whole blood or products derived therefrom.

13. The method as in claim 4 wherein the samples are obtained from young patients selected from the group consisting of embryos, foetuses, neonatals, young infants.

14. The method of claim 4 used to determine subclinical levels of the LSD before onset of physical manifestations become apparent.

15. The method of claim 4 wherein the LSD is Gaucher disease.

16. The method of claim 3 to measure the severity of the LSD.

17. The method of either claim 1 or 3 wherein the LSD is Fabry and a first compound is selected from the group consisting of Cer (ceramide), LC (lactosyl ceramide), CTH (trihexosyceramide) and the second compound is selected from the group consisting of SM (sphingomyelin) and GC (glucosylceramide).

18. The method of claim 17 wherein two or more of Cer, LC and CTH is compared to SM.

19. The method of claim 17 wherein two or more of Cer, LC and CTH is compared to GC.

20. The method of claim 17 wherein the Cer, LC and CTH are C24:1 species.

21. The method of claim 20 wherein CrH and LC (24:1) is compared to SM (C24:0).

22. The method of claim 17 wherein the index is calculated according to the following calculation (LC C24:1*CTH C24:1)/(GC C24:0*SM C24.0).

23. The method of either claim 1 or 3 wherein the LSD is Gaucher and two compounds are selected from the group consisting of SM, LC CTH and the third compound is selected from the group consisting of Cer and GC.

24. The method of claim 23 wherein two or more of SM, LC and CrH are compared to Cer.

25. The method of claim 23 wherein two or more of SM LC and CrH are compared with GC.

26. The method of claim 23 wherein two or more of SM LC and CTH are compared with Cer and GC.

27. A method of developing a diagnostic method comprising the steps of
taking a first group of LSD samples one each from a plurality of LSD individuals affected by one type of LSD,
taking a second group of control samples one each from a plurality of control individuals not affected by LSD
the sample being of a tissue or body fluid of the individual an LSD group of individuals with LSD
interrogating the first group of samples by mass spectrometry for first levels of a plurality of indicators of respective lipid containing storage associated compounds,
interrogating the second group of samples by mass spectrometry for second levels of the plurality of indicators of respective lipid containing storage associated compounds,
the lipid containing storage associated compounds selected from the class of compounds consisting of the group glycolipids and phospholipids,
comparing the first levels with the second levels
identifying a first group of lipid containing storage associated compound which are shown as having increased levels of indicators in the first LSD group compared to the control group,
identifying a second group of lipid containing storage associated compounds which are shows as having decreased levels of indicators in the LSD group compared to the control group,
formulating a combination of two or more of the first and/or second group of indicators by which to calculate and index number whereby to distinguish LSD samples from control samples, and preferably
preparing a standard being a scale of index numbers reflective of the severity of the LSD.
